SEQUENCE LISTING (5 sequences)

SEQ ID NO: 1 - protein, 327 aa, Dickeya chrysanthemi
ADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 2 - protein, 20 aa, synthetic construct
AAPAAPAPAAPAAPAPAAPA

SEQ ID NO: 3 - protein, 348 aa, synthetic construct
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 4 - DNA, 18 bp, synthetic construct
  source 1..18: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = Nucleotide with phosphorothioate linkage
  modified_base 14: mod_base = OTHER; note = Nucleotide with phosphorothioate linkage
  modified_base 15: mod_base = OTHER; note = Nucleotide with phosphorothioate linkage
  modified_base 16: mod_base = OTHER; note = Nucleotide with phosphorothioate linkage
  modified_base 17: mod_base = OTHER; note = Nucleotide with phosphorothioate linkage
tctcccagcgtgcgccat

SEQ ID NO: 5 - protein, 14 aa, synthetic construct
  SITE 1: note = N-terminus amino acid is capped by 4-fluorobenzoyl
  SITE 3: note = 3-(2-naphthyl)-alanine
  SITE 6: note = Citrulline
  SITE 12: note = Citrulline
  SITE 14: note = Amidated residue
  DISULFID 4..13: note = Intrachain disulfide bond
RRXCYXKKPYRXCR

(19) United States
(12) Patent Application Publication
(10) Pub. No.: US 2026/0007728 A1
(43) Pub. Date: Jan. 8, 2026

(54) COMBINATION THERAPIES INVOLVING L-ASPARAGINASE

(71) Applicant: Jazz Pharmaceuticals Ireland Ltd., Dublin (IE)

(72) Inventors: Hemamalini Gursahani, Philadelphia, PA (US); Kedar Shriniwas Vaidya, Philadelphia, PA (US); Alireza Ebrahimnejad, Philadelphia, PA (US)

(21) Appl. No.: 18/880,475

(22) PCT Filed: Jul. 13, 2023
(86) PCT No.: PCT/US2023/027665

§ 371 Date: Dec. 31, 2024

Related U.S. Application Data
(60) Provisional application No. 63/389,327, filed on Jul. 14, 2022.

(51) Int. Cl. A61K 38/50; A61K 31/055; A61K 31/166; A61K 31/351; A61K 31/352; A61K 31/365; A61K 31/366; A61K 31/404; A61K 31/426; A61K 31/428; A61K 31/436; A61K 31/439; A61K 31/444; A61K 31/4725; A61K 31/4741; A61K 31/501; A61K 31/519; A61K 31/566; A61K 31/57; A61K 31/585; A61K 31/635; A61K 31/712; A61P 35/00

(52) U.S. Cl. CPC A61K 38/50; A61K 31/055; A61K 31/166; A61K 31/351; A61K 31/352; A61K 31/365; A61K 31/366; A61K 31/404; A61K 31/426; A61K 31/428; A61K 31/436; A61K 31/439; A61K 31/444; A61K 31/4725; A61K 31/4741; A61K 31/501; A61K 31/519; A61K 31/566; A61K 31/57; A61K 31/585; A61K 31/635; A61K 31/712; A61P 35/00; C12Y 305/01001

(57) ABSTRACT

The present disclosure provides compositions and methods for treating cancer in a human subject comprising dosing a human subject with a monotherapy or combination therapy involving L-asparaginase. Combination therapies disclosed herein include combination therapies with functionalized or unfunctionalized L-asparaginase in combination with a BCL-XL inhibitor, a BCL-2 inhibitor, a CD20 inhibitor, an mTOR inhibitor, or another anticancer agent.

### CROSS-REFERENCE TO RELATED APPLICATIONS

This application claims benefit of priority under 35 U.S.C. § 119(e) of U.S. Provisional Application No. 63/389,327, filed Jul. 14, 2022. The disclosure of the prior application is considered part of and is herein incorporated by reference in the disclosure of this application in their entirety.

### INCORPORATION BY REFERENCE OF SEQUENCE LISTING

The material in the accompanying sequence listing is hereby incorporated by reference into this application. The accompanying sequence listing xml file, name JAZZ1170-1WO_SL.xml Sequence Listing ST26.xml, was created on Jul. 12, 2023 and is 211b.

### TECHNICAL FIELD

The present disclosure provides combination therapies including administration of a L-asparaginase.

### BACKGROUND

Proteins with L-asparagine aminohydrolase activity, commonly known as L-asparaginases, have successfully been used for the treatment of various diseases that are potentially fatal, including cancer, and more particularly, Acute Lymphoblastic Leukemia (ALL) and Lymphoblastic Lymphoma (LBL), for which children constitute a large proportion of patients stricken with these diseases.

There is a need for new and improved dosing regimens for cancer, particularly novel combination dosing regimens. The dosing regimens can include combination therapies involving L-asparaginase and could be used as first line therapy or as a substitute for native E. coli derived L-asparaginase and/or long-acting E. coli derived L-asparaginases when a human subject experiences hypersensitivity. Novel combination therapies involving L-asparaginase, particularly combination therapies involving treatment with L-asparaginase would address a significant medical need (as a component of a multi-agent chemotherapeutic regimen) for patients with a solid tumor, a liquid tumor, ALL, LBL, colorectal cancer (CRC) and acute myeloid leukemia (AML), lymphoma, b cell lymphoma, diffuse B-cell lymphoma (DLBCL), Burkitt lymphoma, lung cancer, small cell lung cancer (SCLC), pancreatic cancer, breast cancer, brain cancer, glioblastoma, astrocytoma, sarcoma, ovarian cancer, and gastric cancer.

### BRIEF SUMMARY OF THE INVENTION

Accordingly, the present disclosure provides for combination therapies involving L-asparaginase.

In one aspect, the present disclosure provides for a method of treating cancer in a patient comprising administering to the patient an effective amount of an L-asparaginase and an inhibitor of BCL-XL. In some embodiments, the BCL-XL inhibitor is selected from the group consisting of: a small molecule that inhibit BCL-XL, an antibody that inhibits BCL-XL, an antibody-drug conjugate with a BCL-XL payload, a dendrimer with a BCL-XL payload, a prodrug that targets BCL-XL, and a proteolysis targeting chimera (PROTAC) that targets BCL-XL. In some embodiments, the BCL-XL inhibitor is selected from the group consisting of: A-1155463, A-1331852, WEHI-539, WEHI-539 HCl, BH31-1, A-1293102, DT2216, XZ424, XZ739, PZ15227, PROTAC 1, and ABBV-155. In some embodiments, the patient has an NRAS mutation. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is a tetramer, and wherein each monomer of the tetramer has a sequence identity of at least 95 percent to SEQ ID NO: 1. In some embodiments, the L-asparaginase is a tetramer, and wherein each monomer of the tetramer comprises SEQ ID NO: 1. In some embodiments, the human subject exhibited hypersensitivity to the E-coli.-derived asparaginase, a PEGylated form thereof, or to an Erwinia asparaginase. In some embodiments, the human subject has terminated E-coli.-derived asparaginase therapy. In some embodiments, the human subject is an adult. In some embodiments, the human subject is pediatric. In some embodiments, the L-asparaginase demonstrates less than 6% aggregation. In some embodiments, the L-asparaginase demonstrates less than 1% aggregation. In some embodiments, the L-asparaginase is non-lyophilized. In some embodiments, the L-asparaginase is recombinantly produced in Pseudomonas fluorescens. In some embodiments, a nadir serum asparaginase activity (NSAA) assay as measured from a serum sample from the human subject equals or exceeds 0.1 IU/mL after administration after treatment with the L-asparaginase. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is Burkitt lymphoma. In some embodiments, the lymphoma is DLBCL. In some embodiments, the lymphoma is b cell lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method comprises using ASNS or BCL2 as a prognostic marker (e.g., as a selection biomarker) to determine treatment. In some embodiments, the method comprises using a Wnt mutation as a prognostic marker to determine treatment.

In another aspect, the present disclosure provides for a method of treating cancer in a patient comprising administering to the patient an effective amount of an L-asparaginase and an inhibitor of BCL-2. In some embodiments, the BCL-2 inhibitor is selected from the group consisting of: a small molecule that inhibits BCL-2, an antibody that inhibits BCL-2, an antibody-drug conjugate with a BCL-2 payload, a dendrimer with a BCL-2 payload, a prodrug that targets BCL-2, and a proteolysis targeting chimera (PROTAC) that targets BCL-2. In some embodiments, the BCL-2 inhibitor is selected from the group consisting of: venetoclax (ABT-199), S55746, BDA-366, oblimersen (G3139), obatoclax, obatoclax mesylate (GX15-070), HA14-1, mifepristone (RU486), TCPOBOP, cinobufagin, nodakenetin (NANI), and motixafortide (BL-8040). In some embodiments, the patient has a MAPK pathway mutation. In some embodiments, the MAPK pathway mutation is an NRAS mutation or a KRAS mutation. In some embodiments, the patient has an NRAS mutation. In some embodiments, the patient has a KRAS mutation. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is a tetramer, and wherein each monomer of the tetramer has a sequence identity of at least 95 percent to SEQ ID NO: 1. In some embodiments, the L-asparaginase is a tetramer, and wherein each monomer of the tetramer comprises SEQ ID NO: 1. In some embodiments, the human subject exhibited hypersensitivity to the E-coli.-derived asparaginase, a PEGylated form thereof, or to an Erwinia asparaginase. In some embodiments, the human subject has terminated E-coli.-derived asparaginase therapy. In some embodiments, the human subject is an adult. In some embodiments, the human subject is pediatric. In some embodiments, the L-asparaginase demonstrates less than 6% aggregation. In some embodiments, the L-asparaginase demonstrates less than 1% aggregation. In some embodiments, the L-asparaginase is non-lyophilized. In some embodiments, the L-asparaginase is recombinantly produced in Pseudomonas fluorescens. In some embodiments, a nadir serum asparaginase activity (NSAA) assay as measured from a serum sample from the human subject equals or exceeds 0.1 IU/mL after administration after treatment with the L-asparaginase. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is Burkitt lymphoma. In some embodiments, the lymphoma is DLBCL. In some embodiments, the lymphoma is b cell lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method comprises using ASNS or BCL2 as a prognostic marker to determine treatment. In some embodiments, the method comprises using a Wnt mutation (e.g., the presence or absence of a Wnt mutation) as a prognostic marker to determine treatment.

In another aspect, the present disclosure provides for a method of treating cancer in a patient comprising administering to the patient an effective amount of an L-asparaginase and an inhibitor of both BCL-XL and BCL-2. In some embodiments, the both BCL-XL and BCL-2 inhibitor is selected from the group consisting of: a small molecule that inhibits both BCL-XL and BCL-2, an antibody that inhibits both BCL-XL and BCL-2, an antibody-drug conjugate with a both BCL-XL and BCL-2 payload, a dendrimer with both a BCL-XL and BCL-2 payload, a prodrug that targets both BCL-XL and BCL-2, and a proteolysis targeting chimera (PROTAC) that targets both BCL-XL and BCL-2. In some embodiments, the inhibitor of both BCL-XL and BCL-2 is selected from the group consisting of: navitoclax (ABT-263), ABT-737, sabutoclax, gossypol, (R)-(−)-gossypol acetic acid, TW-37, gambogic acid, 2-Methoxy-antimycin A, berberine chloride (NSC 646666), berberine chloride hydrate, APG-1252, AZD-0466, BM-1197, AZD4320, and pelcitoclax (APG-1252). In some embodiments, the patient has an NRAS mutation. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is a tetramer, and wherein each monomer of the tetramer has a sequence identity of at least 95 percent to SEQ ID NO: 1. In some embodiments, the L-asparaginase is a tetramer, and wherein each monomer of the tetramer comprises SEQ ID NO: 1. In some embodiments, the human subject exhibited hypersensitivity to the E-coli.-derived asparaginase, a PEGylated form thereof, or to an Erwinia asparaginase. In some embodiments, the human subject has terminated E-coli.-derived asparaginase therapy. In some embodiments, the human subject is an adult. In some embodiments, the human subject is pediatric. In some embodiments, the L-asparaginase demonstrates less than 6% aggregation. In some embodiments, the L-asparaginase demonstrates less than 1% aggregation. In some embodiments, the L-asparaginase is non-lyophilized. In some embodiments, the L-asparaginase is recombinantly produced in Pseudomonas fluorescens. In some embodiments, a nadir serum asparaginase activity (NSAA) assay as measured from a serum sample from the human subject equals or exceeds 0.1 IU/mL after administration after treatment with the L-asparaginase. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is Burkitt lymphoma. In some embodiments, the lymphoma is DLBCL. In some embodiments, the lymphoma is b cell lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method comprises using ASNS or BCL2 as a prognostic marker to determine treatment. In some embodiments, the method comprises using a Wnt mutation as a prognostic marker to determine treatment.

In another aspect, the present invention provides for a method of treating cancer in a patient comprising administering to the patient an effective amount of an L-asparaginase and an mTOR inhibitor. In some embodiments, the mTOR inhibitor is selected from the group consisting of: a small molecule that inhibits mTOR, an antibody that inhibits mTOR, an antibody-drug conjugate with a mTOR payload, a dendrimer with a mTOR payload, a prodrug that targets mTOR, and a proteolysis targeting chimera (PROTAC) that targets mTOR. In some embodiments, the mTOR inhibitor is selected from the group consisting of: rapamycin (sirolimus), rapalogs (rapamycin derivatives), temsirolimus (CCI-779), everolimus (RAD001), and ridaforolimus (AP-23573). In some embodiments, the patient has an NRAS mutation. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is a tetramer, and wherein each monomer of the tetramer has a sequence identity of at least 95 percent to SEQ ID NO: 1. In some embodiments, the L-asparaginase is a tetramer, and wherein each monomer of the tetramer comprises SEQ ID NO: 1. In some embodiments, the human subject exhibited hypersensitivity to the E-coli.-derived asparaginase, a PEGylated form thereof, or to an Erwinia asparaginase. In some embodiments, the human subject has terminated E-coli.-derived asparaginase therapy. In some embodiments, the human subject is an adult. In some embodiments, the human subject is pediatric. In some embodiments, the L-asparaginase demonstrates less than 6% aggregation. In some embodiments, the L-asparaginase demonstrates less than 1% aggregation. In some embodiments, the L-asparaginase is non-lyophilized. In some embodiments, the L-asparaginase is recombinantly produced in Pseudomonas fluorescens. In some embodiments, a nadir serum asparaginase activity (NSAA) assay as measured from a serum sample from the human subject equals or exceeds 0.1 IU/mL after administration after treatment with the L-asparaginase. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the AML has an NRAS mutation. In some embodiments, the cancer is lymphoma. In some embodiments, the lymphoma is Burkitt lymphoma. In some embodiments, the lymphoma is DLBCL. In some embodiments, the lymphoma is b cell lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method comprises using ASNS or BCL2 as a prognostic marker to determine treatment. In some embodiments, the method comprises using a Wnt mutation as a prognostic marker to determine treatment.

In another aspect, the present disclosure provides for a method of treating cancer in a patient comprising administering to the patient an effective amount of an L-asparaginase and an anti-CD20 inhibitor. In some embodiments, the CD20 inhibitor is selected from the group consisting of: a small molecule that inhibits CD20, an antibody that inhibits CD20, an antibody-drug conjugate with a CD20 payload, a dendrimer with a CD20 payload, a prodrug that targets CD20, and a proteolysis targeting chimera (PROTAC) that targets CD20. In some embodiments, the CD20 inhibitor is selected from the group consisting of: rituximab, ofatumumab, ublituximab, ocrelizumab, obinutuzumab, ocaratuzumab, ibritumomab, tiuxetan, tositumomab, TRU-015, IMMU-106, and R-CHOP. In some embodiments, the patient has an NRAS mutation. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is a tetramer, and wherein each monomer of the tetramer has a sequence identity of at least 95 percent to SEQ ID NO: 1. In some embodiments, the L-asparaginase is a tetramer, and wherein each monomer of the tetramer comprises SEQ ID NO: 1.

In some embodiments, the human subject exhibited hypersensitivity to the E-coli.-derived asparaginase, a PEGylated form thereof, or to an Erwinia asparaginase. In some embodiments, the human subject has terminated E-coli.-derived asparaginase therapy. In some embodiments, the human subject is an adult. In some embodiments, the human subject is pediatric. In some embodiments, the L-asparaginase demonstrates less than 6% aggregation. In some embodiments, the L-asparaginase demonstrates less than 1% aggregation. In some embodiments, the L-asparaginase is non-lyophilized. In some embodiments, the L-asparaginase is recombinantly produced in Pseudomonas fluorescens. In some embodiments, a nadir serum asparaginase activity (NSAA) assay as measured from a serum sample from the human subject equals or exceeds 0.1 IU/mL after administration after treatment with the L-asparaginase. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is Burkitt lymphoma. In some embodiments, the lymphoma is DLBCL. In some embodiments, the lymphoma is b cell lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method comprises using ASNS or BCL2 as a prognostic marker to determine treatment. In some embodiments, the method comprises using a Wnt mutation as a prognostic marker to determine treatment. In another aspect, the present disclosure provides for a method of treating cancer in a patient comprising administering to the patient an effective amount of an L-asparaginase and a BTK inhibitor. In some embodiments, the BTK inhibitor is selected from the group consisting of: a small molecule that inhibits BTK, an antibody that inhibits BTK, an antibody-drug conjugate with a BTK payload, a dendrimer with a BTK payload, a prodrug that targets BTK, and a proteolysis targeting chimera (PROTAC) that targets BTK. In some embodiments, the BTK inhibitor is selected from the group consisting of: ibrutinib, zanubrutinib, and acalabrutinib. In some embodiments, the patient has an NRAS mutation. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is a tetramer, and wherein each monomer of the tetramer has a sequence identity of at least 95 percent to SEQ ID NO: 1. In some embodiments, the L-asparaginase is a tetramer, and wherein each monomer of the tetramer comprises SEQ ID NO: 1. In some embodiments, the human subject exhibited hypersensitivity to the E-coli.-derived asparaginase, a PEGylated form thereof, or to an Erwinia asparaginase. In some embodiments, the human subject has terminated E-coli.-derived asparaginase therapy. In some embodiments, the human subject is an adult. In some embodiments, the human subject is pediatric. In some embodiments, the L-asparaginase demonstrates less than 6% aggregation. In some embodiments, the L-asparaginase demonstrates less than 1% aggregation. In some embodiments, the L-asparaginase is non-lyophilized. In some embodiments, the L-asparaginase is recombinantly produced in Pseudomonas fluorescens. In some embodiments, a nadir serum asparaginase activity (NSAA) assay as measured from a serum sample from the human subject equals or exceeds 0.1 IU/mL after administration after treatment with the L-asparaginase. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is Burkitt lymphoma. In some embodiments, the lymphoma is DLBCL. In some embodiments, the lymphoma is b cell lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method comprises using ASNS or BCL2 as a prognostic marker to determine treatment. In some embodiments, the method comprises using a Wnt mutation as a prognostic marker to determine treatment.

In another aspect, the present disclosure provides a method of treating NRAS mutant acute myeloid leukemia (AML) in a patient including administering to the patient an effective amount of an L-asparaginase. In some embodiments, the L-asparaginase is administered as a monotherapy. In some embodiments, the L-asparaginase is administered as part of a combination therapy. In some embodiments, the L-asparaginase is administered in combination with a pan-RAF inhibitor. In some embodiments, the L-asparaginase is unfunctionalized.

In another aspect, the present disclosure provides a method of treating KRAS mutant acute myeloid leukemia (AML) in a patient including administering to the patient an effective amount of an L-asparaginase. In some embodiments, the L-asparaginase is administered as a monotherapy. In some embodiments, the L-asparaginase is administered as part of a combination therapy. In some embodiments, the L-asparaginase is administered in combination with a pan-RAF inhibitor. In some embodiments, the L-asparaginase is unfunctionalized.

In a further aspect, the present invention provides a method of treating solid cancer in a patient including administering to the patient an effective amount of an L-asparaginase. In some embodiments, the L-asparaginase is administered as a monotherapy. In other embodiments, the L-asparaginase is administered with a BCL-XL inhibitor. In particular embodiments, the L-asparaginase is unfunctionalized. In further embodiments, the L-asparaginase is conjugated with a proline- or alanine-containing peptide.

### BRIEF DESCRIPTION OF THE DRAWINGS

The invention can be best understood from the following detailed description when read in conjunction with the accompanying drawings. Included in the drawings are the following figures:

FIG. 1 shows a combination of JZP-458+A-1331852 (BCL-XLi) in vitro. Administration of JZP-458 with a BCL-XL inhibitor (e.g., A-1331852) yielded synergetic cytotoxicity for several different cancer cell lines.

FIG. 2 shows a combination of JZP-458+Venetoclax (BCL-2i) in vitro in a number of different cancer cell lines.

FIG. 3 shows a combination of JZP-458+Navitoclax (BCL-2/BCL-XLi) in vitro in a number of different cancer cell lines.

FIG. 4 shows a combination of JZP-458+CB-839 (glutaminase inhibitor) in vitro in a number of different cancer cell lines.

FIG. 5 shows a combination of JZP-458 and A-1155463 (BCL-XLi) in vitro in a number of different cancer cell lines.

FIG. 6 shows that cell lines derived from multiple tumor indications fall within the same range of sensitivity to JZP-458 in vitro.

FIG. 7 shows combinations of various pathway inhibitors tested for cytotoxicity with JZP-458. Choice of inhibitors was informed by pathway analysis and evaluation of these agents in cell lines with low and high ASNS expression. Data for combinations tested can be found in FIG. 8.

FIG. 8 shows an overview of synergy with cytotoxicity as a readout across various pathway inhibitors+JZP-458 as described in FIG. 7.

FIG. 9 shows a comparison of in vitro synergy of JZP-458 with a BCL-XLi (A-1331852) compared to the combination of JZP-458 with Bortezomib (proteasome inhibitor).

FIGS. 10A, 10B, and 10C show liquid tumors with NRAS mutations appear to be more sensitive to asparaginase. Across the panel sensitivity difference due to NRAS mutations is driven by liquid tumors.

FIG. 11 shows a comparison of BCL2 expression in lymphoma lines post treatment with chemotherapeutic agents. There is significant decrease of BCL2 expression in ABC- and GCB lymphoma lines post treatment with most agents whereas there is significant increase of BCL2 expression in triple hit lymphoma line WSU-DLCL2 post treatment ibrutinib and rapamycin. Decreased BCL2 expression can be used as prognostic marker for DLBCL lines (ABC and GCB type). JZP458 and most chemotherapeutic agents decrease BCL2 expression.

FIG. 12 shows a comparison of ASNS expression in Burkitt lymphoma lines post treatment with chemotherapeutic agents. An alteration of ASNS expression in BJAB, RAJI, RAMOS lines is seen. There is significant increase of ASNS expression post treatment carfilzomib and JZP-458. Increasing BCL2 expression 1.25 fold or 1 fold after treatment with ibrutinib or rapamycin in WSU-DLCL2 and increasing ASNS expression 1.25 fold or 0.75 fold after treatment with carfilzomib or JZP458 both could be indicative that BCL2 and ASNS are prognostic markers for each corresponding treatment. In vitro data show strong sensitivity to carfilzomib in a range of 1.3-7.2 nM for ABC, GCB, and THL lines. As will be appreciated, other proteasome inhibitors may be of use in similar combinations with JZP-458.

FIG. 13 shows synergy of venetoclax with JZP458 in DLBCL line (SU-DHL-2) using the SynergyFinder assay. The average CI50 for all ratios is 0.75. This indicates synergy of venetoclax with JZP458. The SynergyFinder assay shows the synergy of venetoclax with JZP458 in SU-DHL-2 DLBCL line, including overlay curves, isoblograms, and tables presenting IC50 of single compound, IC50 ratios, and combination indices. Combination index (CI) less than 1 demonstrates synergy.

FIG. 14 shows synergy of rapamycin (sirolimus) with JZP-458 in DLBCL line SU-DHL-2 using the SynergyFinder assay, including overlay curves, isoblograms, and tables presenting IC50 of single compound, IC50 ratios and combination indices. Combination index (CI) less than 1 demonstrates synergy.

FIG. 15 shows synergy of rituximab with JZP-458 in triple hit lymphoma line WSU-DLCL2 using the SynergyFinder assay, including overlay curves, and tables presenting IC50 of single compound, IC50 ratios and combination indices. Combination index (CI) less than 1 demonstrates synergy.

FIG. 16 shows a comparison of sensitivity to JZP-458 between DLBCL and Burkitt lymphoma lines. Dose-response curve overlays-72 hrs incubation.

FIG. 17 shows synergy of JZP458 with ibrutinib in lymphoma lines (BLISS matrix).

FIG. 18 shows mean body weight change of naïve SCID mice after treatment with JZP341 (pasylated L-asparaginase) in combination with venetoclax, sirolimus, and constituents of R-CHOP.

FIG. 19 shows body weight change in response to treatment window, doses, schedules, and routes of JZP341 in combination with venetoclax, sirolimus, and constituents of R-CHOP in naïve SCID mice.

FIG. 20 shows a list of cell lines, vendors, cell culture medium, cell number plated in well plate, chemotherapeutic agents used in the experiments described herein.

FIG. 21 shows a list of cell lines, vendors, cell culture medium, cell number plated in well plate used in the experiments described herein.

FIGS. 22A-22D shows a list of examples of BCL-XL inhibitors.

FIGS. 23A-23E shows a list of examples of BCL-2 inhibitors. FIG. 23B discloses SEQ ID NO: 4 and FIG. 23E discloses SEQ ID NO: 5.

FIGS. 24A-24F shows examples of inhibitors of both BCL-2 and BCL-XL.

FIGS. 25A-25B shows a list of examples of mTOR inhibitors.

FIG. 26 shows a list of examples of BTK inhibitors.

FIG. 27 shows an example of a glutaminase inhibitor, CB-839.

FIG. 28 is a plot that shows Bliss synergy scores for pasylated Erwinia chrysanthemi L-asparaginase and A-1331852 against multiple cancer cell lines.

FIG. 29 is a plot that shows Bliss synergy scores for pasylated Erwinia chrysanthemi L-asparaginase and venetoclax against multiple cancer cell lines.

FIG. 30 is a plot that shows Bliss synergy scores for pasylated Erwinia chrysanthemi L-asparaginase and navitoclax against multiple cancer cell lines.

### DETAILED DESCRIPTION OF THE INVENTION

### 1. Overview

The present disclosure provides combination therapies that include L-asparaginase. In particular these combination therapies are used for treating cancer. In certain aspects, the combination therapies include administering an L-asparaginase with one or more of: a B-cell lymphoma-extra large (BCL-XL) inhibitor, a B-cell lymphoma 2 (BCL-2) inhibitor, an inhibitor of both B-cell lymphoma-extra large (BCL-XL) and B-cell lymphoma 2 (BCL-2), an inhibitor of mammalian Target of Rapamycin (mTOR), a CD20 inhibitor, a glutaminase inhibitor, a BTK inhibitor, a protesome inhibitor, a MAP (Mitogen-Activated Protein) Kinase/ERK (Extracellular Signal-Regulated Kinase) Kinase (MEK) inhibitor, a phosphatidylinositol-3 kinase (PI3K) inhibitor, and a dual targeted VEGFR2-TIE2 tyrosine kinase inhibitor. Other therapeutics that can be used in combination with L-asparaginase for the treatment of cancer include chemotherapies such as temozolomide, and FOLFOX (a chemotherapy which includes leucovorin calcium (Folinic acid), fluorouracil, and oxaliplatin).

In further aspects, the combination therapies disclosed herein show a synergistic effect as compared to use of each component of the combination therapy on its own. In certain embodiments, the synergistic effects for the combination therapies described herein are shown using a BLISS assay and/or through experiments providing a Bliss Sum Score.

### II. Definitions

Unless otherwise expressly defined, the terms used herein will be understood according to their ordinary meaning in the art.

As used herein, the term “disease treatable by depletion of asparagine” refers to a condition or disorder wherein the cells involved in or responsible for the condition or disorder either lack or have a reduced ability to synthesize L-asparagine. Depletion or deprivation of L-asparagine can be partial or substantially complete (e.g., to levels that are undetectable using methods and apparatus that are known in the art).

As used herein, the term “therapeutically effective amount” refers to the amount of a protein (e.g., asparaginase or recombinant L-asparaginase thereof), required to produce a desired therapeutic effect.

The term “comprising the sequence of SEQ ID NO:1” means that the amino-acid sequence of the protein is not strictly limited to SEQ ID NO: 1 but can contain additional amino-acids.

The term “subject” or “patient” intends an animal, a mammal, or yet further a human patient.

The term “host cell or a non-human host transformed with the vector” relates to a host cell or a non-human host that comprises the vector or the nucleic acid as described herein. Host cells for the expression of polypeptides are well known in the art and comprise prokaryotic cells as well as eukaryotic cells. Appropriate culture media and conditions for the above described host cells are known in the art.

“Culturing the host or host cell” includes expression of a protein, including as a fusion protein, as defined herein and/or the polypeptide as defined herein and/or of the asparaginase in the host or host cell.

As used herein, the term “about” modifying, for example, the dimensions, volumes, quantity of an ingredient in a composition, concentrations, process temperature, process time, yields, flow rates, pressures, and like values, and ranges thereof, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of, for example, a composition, formulation, or cell culture with a particular initial concentration or mixture, and amounts that differ due to mixing or processing a composition or formulation with a particular initial concentration or mixture. Whether modified by the term “about” the claims appended hereto include equivalents to these quantities. The term “about” further can refer to a range of values that are similar to the stated reference value. In certain embodiments, the term “about” refers to a range of values that fall within 10, 9, 8, 7, 6, 5, 4, 3, 2, 1 percent or less of the stated reference value.

The terms “combination therapy,” “combined administration,” “co-administration,” “co-administering,” “administering,” “administered in combination with,” “administering in combination with,” “simultaneous,” and “concurrent,” as used herein, encompass administration of two or more active pharmaceutical ingredients to a human subject so that both active pharmaceutical ingredients and/or their metabolites are present in the human subject at the same time. These terms and their grammatical equivalents are used interchangeably herein. Co-administration includes simultaneous administration in separate compositions, administration at different times in separate compositions, or administration in a composition in which two or more active pharmaceutical ingredients are present. Simultaneous administration in separate compositions and administration in a composition in which both agents are present is also described herein. Depending on how each one of the active pharmaceutical ingredients is delivered the frequency of dosing may vary. For example, a combination therapy with two or more drugs can include dosing the two or more drugs on different days to achieve a particular overlap of pharmacokinetic exposures. In some embodiments, each or both of the active pharmaceutical ingredients can be given every one day, two days, three days, four days, five days, six days, one week, two weeks, three weeks, 1 month, 2 months, or three months. In some embodiments, an active pharmaceutical ingredient is administered following administration with L-asparaginase. In some embodiments, an active pharmaceutical ingredient is administered before administration with L-asparaginase. In some embodiments, there are at least 3-6 h between the administration of each one of the active pharmaceutical ingredients. In some embodiments there is one, two, three, four, five, six, seven, eight, nine, ten, eleven, twelve hours between the administration of each one of the active pharmaceutical ingredients. In some embodiments there are about 24 hours between the administration of each one of the active pharmaceutical ingredients. In some embodiments there are about 48 hours between the administration of each one of the active pharmaceutical ingredients. Administration of each one of the active pharmaceutical ingredients can be by many different routes, including, but not limited to: intravenous, intraperitoneal, subcutaneous, intramuscular, oral, topical (transdermal), or transmucosal administration.

As used herein, the term “therapeutically effective amount” refers to the amount of a protein (e.g., recombinant L-asparaginase or conjugate thereof), required to produce a desired therapeutic effect.

The terms “E. coli-derived L-asparaginase,” “L-asparaginase from E. coli,” “E. coli asparaginase,” and “E. coli L-asparaginase” are used interchangeably to refer to an asparaginase that is natively produced in E. coli.

The terms “Erwinia-derived L-asparaginase,” “Erwinia asparaginase,” “Erwinia L-asparaginase,” “Erwinia asparaginase,” “L-asparaginase from Erwinia,” and “asparaginase from Erwinia,” are used interchangeably herein to refer to an asparaginase that is natively produced in Erwinia.

The terms “L-asparaginase from Erwinia chrysanthemi,” “Erwinia chrysanthemi L-asparaginase” and “Erwinia chrysanthemi-derived L-asparaginase” are used interchangeably to refer to an asparaginase that is natively produced in Erwinia chrysanthemi. Erwinia chrysanthemi (also known as Pectobacterium chrysanthemi) has been renamed Dickeya chrysanthemi. Thus, the terms Erwinia chrysanthemi, Pectobacterium chrysanthemi and Dickeya chrysanthemi are used interchangeably herein.

Erwinaze® (Biologic License Application 125359) is an Erwinia chrysanthemi L-asparaginase type II product commercially approved in the United States for treatment of ALL in patients. Its active ingredient is Erwinia chrysanthemi L-asparaginase type II (see Erwinaze® package insert, incorporated herein by reference).

The terms “homology” and “sequence identity” are used interchangeably herein. Percent (%) amino acid sequence identity with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs to of US Pub. No. 20160244525, hereby incorporated by reference. The degree of identity between an amino acid sequence of the present disclosure (“disclosure sequence”) and the parental amino acid sequence is calculated as the number of exact matches in an alignment of the two sequences, divided by the length of the “disclosure sequence,” or the length of the parental sequence, whichever is the shortest. The result is expressed in percent identity. In some embodiments, two or more amino acid sequences are at least 50%, 60%, 70%, 80%, or 90% identical. In some embodiments, two or more amino acid sequences are at least 95%, 97%, 98%, 99%, or even 100% identical.

As used herein, the term “Bliss Assay” or “Bliss sum score” refers to a method of determining synergy for combination treatments. Examples of methods for determining synergy using a Bliss Assay can be found in, but are not limited to, Liu Q et al., Evaluation of drug combination effect using a Bliss independence dose-response surface model. Stat Biopharm Res 2018, 10:112-22 and Leverson J D et al., Exploiting selective BCL-2 family inhibitors to dissect cell survival dependencies and define improved strategies for cancer therapy. Sci Transl Med 2015, 7:279ra40, which are incorporated by reference herein. A Bliss assay protocol is also outlined in Example 1. A Bliss score of less than zero is considered antagonism, a Bliss score equal to zero is considered additivity, and a A positive Bliss Sum score greater than 1 is indicative of synergy, with a score >150 being considered strong synergy (See Leverson et al., Exploiting selective Bc1-2 family inhibitors to dissect cell survival dependencies and define improved strategies for cancer therapy, Sci Transl Med. (201) 7:279, Tan et al., Navitoclax Enhances the Efficacy of Taxanes in Non-Small Cell Lung Cancer Models). As will be appreciated, scores between 1 and 150 and scores above 150 are also useful indicators of synergistic combinations.

As used herein, the term “SynergyFinder assay” refers to a specific assay that is used to test synergy (see further description in Example 1). A combination index (CI) evaluates the concentrations needed to achieve a fixed-effect. less than 1 demonstrates synergy. A CI of below 1 indicates synergy. A CI of less than 0.3 indicates strong synergy. In some embodiments, a CI of 0.1 indicates that the combination needs a ten-fold lower concentration than expected from the single agent data to achieve the same effect level. In some embodiments, when a potent and less potent compound with a CI of 0.1 are combined, the effective concentration of the potent compound is improved tenfold by the less potent compound.

As used herein, the term “ABC-type” stands for Activated B cell type and the term “GBC-type” stands for Germinal center b cell type.

As used herein, the term “double hit” is defined by two recurrent chromosome translocations; MYC/8q24 loci, usually in combination with the t (14; 18) (q32; q21) bcl-2 gene or/and BCL6/3q27 chromosomal translocation.

As used herein, the term “triple hit” is defined by three recurrent chromosome translocations; MYC/8q24 loci, usually in combination with the t (14; 18) (q32; q21) bcl-2 gene or/and BCL6/3q27 chromosomal translocation.

### III. L-asparaginase

As will be described herein, combination therapies encompassed by the present disclosure include L-asparaginase as part of those combination therapies. Any of the L-asparaginases described herein and known in the art can be used in accordance with the compositions and methods provided herein.

L-asparaginases of bacterial origin have a high immunogenic and antigenic potential. These products can provoke adverse hypersensitivity reactions including allergic reaction, silent inactivation, and anaphylactic shock in patients. L-asparaginases are enzymes with L-asparagine aminohydrolase activity. L-asparaginase enzymatic activity can include not only deamidation of asparagine to aspartic acid and ammonia, but also deamidation of glutamine to glutamic acid and ammonia (also referred to as “glutamine depeletion”). L-asparaginases from E. coli and Erwinia chrysanthemi are commonly used to treat a variety of diseases treated by asparagine depletion, including ALL and LBL. While healthy cells can produce asparagine, some diseased cells are unable to produce asparagine as they lack asparagine synthetase. When an L-asparaginase is administered to a diseased patient, the L-asparaginase reduces the levels of soluble asparagine, starving the diseased cells but not the healthy cells and leading to selective diseased cell death.

In one aspect, a L-asparaginase in accordance with the disclosure provided herein is a recombinant L-asparaginase. In a further aspect, a L-asparaginase in accordance with the present disclosure is an enzyme with L-asparagine aminohydrolase activity. Such a L-asparaginase's enzymatic activity can include not only deamidation of asparagine to aspartic acid and ammonia, but also deamidation of glutamine to glutamic acid and ammonia.

In some embodiments, a L-asparaginase as disclosed herein is active as a multimer. In some embodiments, the L-asparaginase is an active enzyme as a tetramer. A tetramer is composed of four subunits (also known as monomers). In some embodiments, a L-asparaginase is a tetramer consisting of four identical 35 kD subunits. In some embodiments, the L-asparaginase is a non-disulfide bonded tetrameric therapeutic protein. In a particular embodiment, each of the subunits or monomers of a multimeric L-asparaginase comprises the amino acid sequence of SEQ ID NO: 1. In a particular embodiment, each of the subunits or monomers of a tetrameric L-asparaginase comprises the amino acid sequence of SEQ ID NO: 1. In another embodiment, the L-asparaginase is from Erwinia chrysanthemi NCPPB 1066 (Genbank Accession No. CAA32884, incorporated herein by reference in its entirety), either with or without signal peptides and/or leader sequences. In some embodiments, the L-asparaginase is JZP-458 (or JZP458). In some embodiments, the L-asparaginase is a long-acting L-asparaginase. In some embodiments, the long-acting L-asparaginase is JZP-341 (JZP341).

In some embodiments, the L-asparaginase is composed of multiple subunits, for example, four subunits or monomers (tetramer). In some embodiments, the L-asparaginase is a recombinant L-asparaginase composed of multiple subunits, for example, four subunits or monomers (tetramer). A corresponding modified recombinant protein can then, e.g., consist of 1 to 20 (or more) peptides conjugated to each of the monomers of that tetramer. In some embodiments, the recombinant L-asparaginase comprises a monomer and 1, 2, 3, 4, 5, 6, 7, 8, 9, 10, 11, 12, 13, 14, 15, 16, 17, 18, 19, and 20 (or more) peptides conjugated to each of the L-asparaginase monomers. In a specific embodiment, the L-asparaginase is a multimer comprising multiple subunits or monomers, such as a tetramer, and each of the monomers in that tetramer is conjugated to 1 peptide, resulting in a tetramer comprising 4 conjugated peptides, one for each monomer. In some embodiments, the L-asparaginase is a tetramer comprising 1-4 peptides conjugated to each of the L-monomers. In some embodiments, the L-asparaginase is a tetramer comprising 4-20 peptides conjugated to each of the L-monomers. In some embodiments, the L-asparaginase is a tetramer comprising 6-18 peptides conjugated to each of the L-monomers. In some embodiments, the L-asparaginase is a tetramer comprising 6-18 peptides conjugated to each of the L-monomers. In some embodiments, the L-asparaginase is a tetramer comprising 10-15 peptides conjugated to each of the L-monomers.

In one aspect, the present disclosure relates to a modified protein having a L-asparaginase and multiple chemically attached peptide sequences. In a further aspect the length of the peptide sequences are from about 10 to about 100, from about 15 to about 60 or from about 20 to about 40.

Fragments of L-asparaginase, preferably fragments of the recombinant L-asparaginase of SEQ ID NO:1, can be of use in the present disclosure. The term “a fragment of recombinant L-asparaginase” (e.g., a fragment of the recombinant L-asparaginase of SEQ ID NO: 1) means that the sequence of the recombinant L-asparaginase can include fewer amino-acids than in the recombinant L-asparaginases exemplified herein (e.g. the recombinant L-asparaginase of SEQ ID NO: 1) but still enough amino-acids to confer L-aminohydrolase activity. For example, a “fragment of recombinant L-asparaginase” is a fragment that is/consists of at least about 150 or 200 contiguous amino acids of one of the recombinant L-asparaginases exemplified herein (e.g. the recombinant L-asparaginase of SEQ ID NO: 1) (for example about 150, 160, 170, 180, 190, 200, 210, 220, 230, 240, 250, 260, 270, 280, 290, 300, 310, 320, 321, 322, 323, 324, 325, 326 contiguous amino acids) and/or wherein said fragment has up to 50 amino acids deleted from the N-terminus of said recombinant L-asparaginases exemplified herein (e.g. the recombinant L-asparaginase of SEQ ID NO: 1) (e.g. up to 1, 2, 3, 4, 5, 6, 7, 8, 9, 10, 15, 20, 25, 30, 35, 40, 45, or 50) and/or has up to up to 75 or 100 amino acids deleted from the C-terminus of said recombinant L-asparaginases exemplified herein (e.g. the recombinant L-asparaginase of SEQ ID NO: 1) (e.g. up to 1, 2, 3, 4, 5, 6, 7, 8, 9, 10, 15, 20, 25, 30, 35, 40, 45, 50, 55, 60, 70, 75, 80, 85, 90, 95 or 100) and/or has deleted amino acids at both the N-terminus and the C-terminus of said recombinant L-asparaginases exemplified herein (e.g. the recombinant L-asparaginase of SEQ ID NO: 1), wherein the total number of amino acids deleted can be up to 125 or 150 amino acids.

Indeed, a person skilled in the art will understand how to select and design homologous proteins retaining substantially their L-asparaginase activity. Typically, a Nessler assay is used for the determination of L-asparaginase activity according to a method described by Mashburn and Wriston (Mashburn, L., and Wriston, J. (1963) “Tumor Inhibitory Effect of L-Asparaginase,”Biochem Biophys Res Commun 12, 50, incorporated herein by reference in its entirety).

It is well known in the art that a polypeptide can be modified by substitution, insertion, deletion and/or addition of one or more amino acids while retaining its enzymatic activity. The term “one or more amino acids” in this context can refer to one, two, three, four, five, six, seven, eight, nine, ten or more amino acids. For example, substitution of one amino-acid at a given position by a chemically equivalent amino-acid that does not affect the functional properties of a protein is common Substitutions can be defined as exchanges within one of the following groups:
Small aliphatic, non-polar or slightly polar residues: Ala, Ser, Thr, Pro, Gly
Polar, negatively charged residues and their amides: Asp, Asn, Glu, Gln
Polar, positively charged residues: His, Arg, Lys
Large aliphatic, non-polar residues: Met, Leu, Ile, Val, Cys
Large aromatic residues: Phe, Tyr, Trp.

Thus, changes that result in the substitution of one negatively charged residue for another (such as glutamic acid for aspartic acid) or one positively charged residue for another (such as lysine for arginine) can, in certain instances, be expected to produce a functionally equivalent product.

The positions where the amino-acids are modified and the number of amino-acids that can be modified in the amino-acid sequence are not particularly limited. The skilled artisan is able to recognize the modifications that can be introduced without affecting the activity of the protein. For example, modifications in the N- or C-terminal portion of a protein may be expected not to alter the activity of a protein under certain circumstances. With respect to asparaginases, in particular, much characterization has been done, particularly with respect to the sequences, structures, and the residues forming the active catalytic site. This provides guidance with respect to residues that can be modified without affecting the activity of the enzyme. All known L-asparaginases from bacterial sources have common structural features. All are homotetramers with four active sites between the N- and C-terminal domains of two adjacent monomers (Aghaipour (2001) Biochemistry 40, 5655-5664, incorporated herein by reference in its entirety). All have a high degree of similarity in their tertiary and quaternary structures (Papageorgiou (2008) FEBS J. 275, 4306-4316, incorporated herein by reference in its entirety). The sequences of the catalytic sites of L-asparaginases are highly conserved between Erwinia chrysanthemi, Erwinia carotovora, and E. coli L-asparaginase II (Id). The active site flexible loop contains amino acid residues 14-33, and structural analysis show that Thr15, Thr95, Ser62, Glu63, Asp96, and Ala120 contact the ligand (Id). Aghaipour et al. have conducted a detailed analysis of the four active sites of Erwinia chrysanthemi L-asparaginase by examining high resolution crystal structures of the enzyme complexed with its substrates (Aghaipour (2001) Biochemistry 40, 5655-5664). Kotzia et al. provide sequences for L-asparaginases from several species and subspecies of Erwinia and, even though the proteins have only about 75-77% identity between Erwinia chrysanthemi and Erwinia carotovora, they each still have L-asparaginase activity (Kotzia (2007) J. Biotechnol. 127, 657-669). Moola et al performed epitope mapping studies of Erwinia chrysanthemi 3937 L-asparaginase and were able to retain enzyme activity even after mutating various antigenic sequences in an attempt to reduce immunogenicity of the asparaginase (Moola (1994) Biochem. J. 302, 921-927). In view of the extensive characterization that has been performed on L-asparaginases, one of skill in the art could determine how to make fragments and/or sequence substitutions while still retaining enzyme activity.

More specifically, fragments of the protein of SEQ ID NO: 1 are also comprised within the definition of the protein used in the recombinant L-asparaginase described herein. The term “a fragment of SEQ ID NO: 1” means that the sequence of the polypeptide can include fewer amino-acids than the full-length SEQ ID NO: 1 but retains enough of the protein to confer L-aminohydrolase activity. In some embodiments, a recombinant L-asparaginase has at least about 80% homology or identity with the protein comprising SEQ ID NO: 1. In some embodiments, a recombinant L-asparaginase comprises a sequence identity of at least about 85%, 86%, 87%, 88%, 89%, 90%, 91%, 92%, 93%, 94%, 95%, 96%, 97%, 98%, or 99% identity to the amino acid sequence of SEQ ID NO: 1. The terms “homology” and “sequence identity” are used interchangeably herein. The term “comprising the sequence of SEQ ID NO: 1” (for example if the L-asparaginase has 100% homology or sequence identity to the amino acid sequence of SEQ ID NO: 1) means that the amino acid sequence of the asparaginase can not be strictly limited to SEQ ID NO: 1 but can contain one, two, three, four, five, six, seven, eight, nine, ten or more additional amino acids.

SEQ ID NO: 1 is as follows:

ADKLPNIVILATGGTIAGSAATGTOTTGYKAGALGVDTLINAVPE
VKKLANVKGEQFSNMASENMTGDVVLKLSQRVNELLARDDVDGVV
ITHGTDTVEESAYFLHLTVKSDKPVVEVAAMRPATAISADGPMNL
LEAVRVAGDKQSRGRGVMVVLNDRIGSARYITKTNASTLDTFKAN
EEGYLGVIIGNRIYYQNRIDKLHTTRSVFDVRGLTSLPKVDILYG
YQDDPEYLYDAAIQHGVKGIVYAGMGAGSVSVRGIAGMRKAMEKG
VVVIRSTRTGNGIVPPDEELPGLVSDSLNPAHARILLMLALTRTS
DPKVIQEYFHTY

The present disclosure also relates to a nucleic acid encoding the recombinant L-asparaginase described herein, particularly a nucleic acid encoding SEQ ID NO: 1 as defined herein.

### A. PEGylation

In certain aspects, the L-asparaginase as described herein further comprises and/or is conjugated to a polymer. In some embodiments, the L-asparaginase as described herein is conjugated with a polyethylene glycol (PEG) moiety. In other embodiments, the L-asparaginase is not conjugated with a PEG moiety. In some embodiments, the L-asparaginase is JZP-715.

In some embodiments, polymers are selected from the group of non-toxic water soluble polymers such as polysaccharides, e.g. hydroxyethyl starch, poly amino acids, e.g. poly lysine, polyester, e.g., polylactic acid, and poly alkylene oxides, e.g., polyethylene glycol (PEG). Polyethylene glycol (PEG) or mono-methoxy-polyethyleneglycol (mPEG) is well known in the art and comprises linear and branched polymers. Examples of some polymers, particularly PEG, are provided in the following, each of which is herein incorporated by reference in its entirety: U.S. Pat. Nos. 5,672,662; 4,179,337; 5,252,714; U.S. Patent Application Publication No. 2003/0114647; U.S. Pat. Nos. 6,113,906; 7,419,600; 9,920,311 PCT Publication WO2019/109018, and PCT Publication No. WO2004/083258.

The quality of such polymers is characterized by the polydispersity index (PDI). The PDI reflects the distribution of molecular weights in a given polymer sample and is calculated from the weight average molecular weight divided by the number average molecular weight. It indicates the distribution of individual molecular weights in a batch of polymers. The PDI has a value always greater than 1, but as the polymer chains approach the ideal Gauss distribution (=monodispersity), the PDI approaches 1.

In some embodiments, L-asparaginase is conjugated to a PEG or mPEG molecule. The PEG or mPEG can have a molecular weight within the range of about 500 Da to 15,000 Da, about 1,000 to 10,000 Da, about 1,500 to 7,500 Da, about 3,000 to 7,000 Da, about 4,000 to 6,000 Da, or about 4,500 to 5,500 Da. For example, the PEG or mPEG can have a molecular weight of about 500 Da, about 1,000 Da, about 1,500 Da, about 2,000 Da, about 2,500 Da, about 3,000 Da, about 3,500 Da, about 4,000 Da, about 4,500 Da, about 5,000 Da, about 5,500 Da, about 6,000 Da, about 6,500 Da, about 7,000 Da, about 7,500 Da, about 8,000 Da, about 8,500 Da, about 9,000 Da, about 9,500 Da, about 10,000 Da, about 10,500 Da, about 11,000 Da, about 11,500 Da, about 12,000 Da, about 12,500 Da, about 13,000 Da, about 13,500 Da, about 14,000 Da, about 14,500 Da, or about 15,000 Da, for example. In some embodiments, the PEG or mPEG has a molecular weight of about 5,000 Da.

In some embodiments, a monomer of the L-asparaginase is coupled to between about 2 and 18 polymers, between about 4 and 16 polymers, between about 6 and 14 polymers, between about 7 and 13 polymers, or between about 8 and 12 polymers (e.g., a population of L-asparaginase monomers are coupled to an average of 8 to 12 polymers). In some embodiments, the polymer is PEG or mPEG.

In one embodiment, the conjugate has the formula: Asp-[NH—CO—CH₂)x-CO—NH—PEG]n, wherein Asp is the recombinant L-asparaginase, NH is one or more of the NH groups of the lysine residues and/or the N-terminus of the Asp, PEG is a polyethylene glycol moiety, n is a number that represents at least about 40% to about 100% of the accessible amino groups (e.g., lysine residues and/or the N-terminus) in the Asp, and x is an integer ranging from about 1 to about 8, more specifically, from about 2 to about 5. In a specific embodiment, the PEG is monomethoxy-polyethylene glycol (mPEG).

### B. PASylation

In some embodiments, the L-asparaginase is conjugated with a proline- and alanine-containing peptide. In some embodiments, the L-asparaginase is conjugated with a proline- or alanine-containing peptide. In other embodiments, the recombinant crisantaspase is not conjugated with a proline-, alanine-, or serine-containing peptide. In such cases, the L-asparaginase can be referred to as “PASylated”, denoting its attachment to the proline and/or alanine containing peptide. In some embodiments, the L-asparaginase is JZP-341.

In some embodiments, the L-asparaginase is a fusion protein with (i) a recombinant L-asparaginase and (ii) one or more polypeptide(s) that are primarily composed of proline, alanine, and serine. In some embodiments, the L-asparaginase is a fusion protein with (i) a recombinant L-asparaginase and (ii) one or more polypeptide(s) that are primarily composed of proline and alanine. In some embodiments, the recombinant L-asparaginase has at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.5% or about 100% identity to the amino acid sequence of SEQ ID NO: 1. In some embodiments, the recombinant L-asparaginase comprises SEQ ID NO:1. In some embodiments, the recombinant L-asparaginase is SEQ ID NO:1.

The one or more polypeptide(s) can be random coil polypeptides. Random coil polypeptides can be characterized by weak intramolecular interactions that favor interconversion between an ensemble of structures, rendering them less prone to generate antibody responses than polypeptides that adopt stable, structurally defined conformations. Nonetheless, fusion to a random coil polypeptide (e.g., the one or more polypeptide(s) with proline, alanine, and optionally serine) can greatly increase the stability, hydrodynamic radius, and circulating half-life (e.g., by disfavoring biliary clearance) of a protein. While random coil peptide functionalization can decrease the activity of certain enzymes, for example by sterically blocking substrate access to an active site, L-asparaginase activity can be enhanced by pasylation, and in particular by pasylation with proline- and alanine-containing polypeptides with about 100 to 600 amino acids.

In some embodiments, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% of amino acid residues of the one or more polypeptide(s) are proline, alanine, or serine. In some embodiments, at least about 50%, at least about 55%, at least about 60%, at least about 65%, at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% of amino acid residues of the one or more polypeptide(s) are proline or alanine. In some embodiments, the one or more polypeptide(s) has at least one serine. In some embodiments, the one or more polypeptide(s) does not include serine. In some embodiments, the one or more polypeptide(s) consist of proline and alanine.

In some embodiments, the one or more polypeptide(s) are each between about 20 and 1200 amino acids in length. For example, the one or more polypeptide(s) can each be between about 20 and 50, between about 20 and 100, between about 50 and 100, between about 50 and 200, between about 50 and 400, between about 100 and 200, between about 100 and 400, between about 100 and 500, between about 100 and 600, between about 150 and 450, between about 200 and 400, between about 200 and 600, between about 200 and 800, between about 400 and 800, or between about 600 and 1200 amino acids in length. In particular embodiments, the one or more polypeptides are each between about 100 and 600, between about 150 and 450, or between about 200 and 400 amino acids in length.

In some embodiments, the recombinant L-asparaginase is conjugated to a single polypeptide. The polypeptide can be coupled to the N- or C-terminus of the recombinant L-asparaginase, through an isopeptide bond (e.g., to a lysine of the recombinant L-asparaginase), or through a linker, such as a succinimide or chemically-derived ester. In particular embodiments, the L-asparaginase is a single expression construct with the polypeptide coupled to the N- or C-terminus of the recombinant L-asparaginase through a peptide bond.

In many embodiments, the one or more polypeptide(s) do not disrupt the activity of the recombinant L-asparaginase. For example, in many embodiments, the recombinant L-asparaginase has at least about 50%, at least about 55%, at least about 60%, at least about 65%, at least about 70%, at least about 75%, at least about 80%, at least about 85%, at least about 90%, at least about 95%, at least about 98%, or at least about 99% of its activity when coupled to the one or more polypeptide(s) (as compared to the recombinant L-asparaginase not coupled to the one or more polypeptide(s)).

In some embodiments, the L-asparaginase is a fusion protein comprising (i) a recombinant L-asparaginase having at least 85%, 90%, 91%, 92%, 93%, 94%, 95%, 96%, 97%, 98%, 99%, or 100% identity to the amino acid sequence of SEQ ID NO: 1 and (ii) one or more polypeptide(s), wherein the polypeptide consists solely of proline and alanine amino acid residues.

In such fusion proteins, the proline residues in the polypeptide consisting solely of proline and alanine amino acid residues can constitute more than about 10% and less than about 70% of the polypeptide. Accordingly, in such fusion proteins, it can be preferred that 10% to 70% of the total number of amino acid residues in the polypeptide are proline residues; more preferably, 20% to 50% of the total number of amino acid residues comprised in the polypeptide are proline residues; and even more preferably, 30% to 40% (e.g., 30%, 35% or 40%) of the total number of amino acid residues comprised in the polypeptide are proline residues. The polypeptide can comprise a plurality of amino acid repeats, wherein said repeat consists of proline and alanine residues and wherein no more than 6 consecutive amino acid residues are identical. Particularly, the polypeptide can comprise or consist of the amino acid sequence AAPAAPAPAAPAAPAPAAPA (SEQ ID NO: 2) or circular permuted versions or (a) multimers(s) of the sequences as a whole or parts of the sequence. In other embodiments, the L-asparaginase specifically lacks such a polypeptide, e.g., the L-asparaginase is not conjugated to a polypeptide containing the above-described percentages or repeats of proline residues.

The present disclosure also relates to a nucleic acid encoding the L-asparaginase, particularly a fusion protein as defined herein. In some embodiments, the nucleotide sequence is a sequence encoding any of the L-asparaginases comprising SEQ ID NO: 1 and a polypeptide, wherein the polypeptide consists solely of proline and alanine amino acid residues, preferably wherein the protein is a fusion protein, described herein, except that one or more amino acid is added, deleted, inserted or substituted, with the proviso that the fusion protein having this amino acid sequence retains L-asparaginase activity. In other embodiments, the nucleotide sequence is a sequence encoding any L-asparaginase comprises SEQ ID NO: 1, wherein that sequence is not conjugated to (or part of a sequence encoding a fusion protein that contains) a polypeptide that consists solely of proline and alanine amino acid residues.

The L-asparaginase according to the present disclosure can be prepared using methods known in the art, particularly those methods disclosed in U.S. Pat. No. 10,174,302 and PCT Application No. WO2019/109018, herein incorporated by reference for exemplary embodiments.

### C. Compositions Comprising L-asparaginase

The present disclosure also provides for compositions comprising a L-asparaginase. Such compositions can include a L-asparaginase in combination with other elements (including without limitation buffers, salts, and excipients). Such compositions can include vehicles for administering L-asparaginase into a subject, including for example particles, powders, and encapsulation.

In some embodiments, a L-asparaginase described herein can be encapsulated. The encapsulation of L-asparaginase in erythrocytes can in some instances serve to improve the therapeutic index (D. Schrijvers et al., Clin. Pharmacokinet. 2003, 42 (9): 779-791). Methods for encapsulation are described for example in EP1773452, which is incorporated by reference herein in its entirety and in particular for all teachings related to encapsulation of L-asparaginase.

### D. Functional Aspects and Other Characteristics of Combination Therapies Involving L-asparaginase and Compositions Thereof.

As will be appreciated, discussion herein of functional aspects and other characteristics of combination therapies involving recombinant L-asparaginase (e.g., an unfunctionalized recombinant L-asparaginase, a pegylated recombinant L-asparaginase, or a pasylated recombinant L-asparaginase) can also apply to compositions comprising combination therapies involving the recombinant L-asparaginase of the presently disclosure.

In some aspects, a L-asparaginase described herein can elicit a lower immunogenic response in the patient as compared to a wild-type L-asparaginase. In some embodiments, the recombinant L-asparaginase described herein can have a greater AUC value after a single dose compared to the native L-asparaginase. These characteristics of the recombinant L-asparaginase described herein are beneficial for a patient that can have a previous hypersensitivity to an E. coli L-asparaginase or a PEGylated form thereof. In some embodiments, the recombinant L-asparaginase described herein does not raise any significant antibody response for a particular period of time after administration of a single dose, e.g., greater than about 1 week, 2 weeks, 3 weeks, 4, weeks, 5 weeks, 6 weeks, 7 weeks, 8 weeks, 9 weeks, 10 weeks, 11 weeks, 12 weeks, or longer. In one example, “does not raise any significant antibody response” means that the subject receiving the recombinant L-asparaginase is identified within art-recognized parameters as antibody-negative. Antibody levels can be determined by methods known in the art, for example ELISA or surface plasmon resonance assays (Zalewska-Szewczyk (2009) Clin. Exp. Med. 9, 113-116; Avramis (2009) Anticancer Research 29, 299-302, each of which is incorporated herein by reference in its entirety).

Compositions comprising the recombinant L-asparaginase of the present disclosure display reduced aggregation compared to those containing Erwinase® and Erwinia chrysanthemi L-asparaginase recombinantly expressed in E. coli. In some embodiments, compositions comprising the recombinant L-asparaginase described herein demonstrates reduced aggregation compared to compositions containing other forms L-asparaginase. For example, processes for manufacturing an unconjugated recombinant L-asparaginase of the present disclosure result in lower aggregation than Erwinase® and Erwinia chrysanthemi L-asparaginase recombinantly expressed in E. coli. The process for making batches of Erwinase® for example, results in a product with about 6% aggregation. Batches of a recombinant L-asparaginase of the present disclosure generally have less than about 1% aggregation.

In some embodiments, the recombinant L-asparaginase of the disclosure has greater purity than other L-asparaginases. In some embodiments, purity is measured by demonstrating the amount of aggregation in a given sample of an asparaginase. The amount of aggregation can be demonstrated by various methods described in the art, including but not limited to Size-Exclusion Chromatogaphy (SEC-HPLC), Size-Exclusion Ultrahigh-Performance Liquid Chromatography (SE-UHPLC), Size Exclusion Chromatography-Multi-Angle Light Scattering (SEC MALLS), and sedimentation velocity Analytical Ultracentrifugation (svAUC). In some embodiments, the amount of aggregation of the recombinant L-asparaginase is less than 10%, 9%, 8%, 7%, 6%, 5%, 4%, 3%, 2%, 1%, 0.5%, 0.25%, 0.2%, 0.1%, or 0.01%. In some embodiments, the amount of aggregation seen in compositions containing the recombinant L-asparaginase is less than 1-10%. In some embodiments, the amount of aggregation seen in compositions containing the recombinant L-asparaginase is less than 10%. In some embodiments, the amount of aggregation of the recombinant L-asparaginase is less than 9%. In some embodiments, the amount of aggregation of the recombinant L-asparaginase is less than 8%. In some embodiments, the amount of aggregation of the recombinant L-asparaginase is less than 7%. In some embodiments, the amount of aggregation of the recombinant L-asparaginase is less than 6%. In some embodiments, the amount of aggregation of the recombinant L-asparaginase is less than 5%. In some embodiments, the amount of aggregation of the recombinant L-asparaginase is less than 4%. In some embodiments, the amount of aggregation of the recombinant L-asparaginase is less than 3%. In some embodiments, the amount of aggregation of the recombinant L-asparaginase is less than 2%. In some embodiments, the amount of aggregation of the recombinant L-asparaginase is less than 1%. In some embodiments, the amount of aggregation of the recombinant L-asparaginase is less than 0.5%. In some embodiments, the amount of aggregation of the recombinant L-asparaginase is less than 0.25%. In some embodiments, the amount of aggregation of the recombinant L-asparaginase is less than 0.2%. In some embodiments, the amount of aggregation of the recombinant L-asparaginase is less than 0.1%. In some embodiments, the amount of aggregation of the recombinant L-asparaginase is less than 0.01%. In some embodiments, the amount of aggregation of the recombinant L-asparaginase is between 0.01% and 10%. In some embodiments, the amount of aggregation of the recombinant L-asparaginase is between about 0.01% and about 9%. In some embodiments, the amount of aggregation of the recombinant L-asparaginase is between about 0.01% and about 8%. In some embodiments, the amount of aggregation of the recombinant L-asparaginase is between about 0.01% and about 7%. In some embodiments, the amount of aggregation of the recombinant L-asparaginase is between about 0.01% and about 6%. In some embodiments, the amount of aggregation of the recombinant L-asparaginase is between about 0.1% and about 5%. In some embodiments, the amount of aggregation of the recombinant L-asparaginase is between about 0.2% and about 4%. In some embodiments, the amount of aggregation of the recombinant L-asparaginase is between about 0.25% and about 3%. In some embodiments, the amount of aggregation of the recombinant L-asparaginase is between about 0.5% and about 2%. In some embodiments, the amount of aggregation of the recombinant L-asparaginase is about 1%. In some embodiments, the amount of aggregation of the recombinant L-asparaginase is 1%.

It is known to those skilled in the art that lower amounts of aggregation typically results in a product with lower immunogenicity. Immunogenicity is a key factor in causing adverse events in the clinic and is regulated by the Federal Drug Administration (FDA) (See U.S. Department of Health and Human Services, Guidance for Industry: Immunogenicity Assessment for Therapeutic Protein Products, 2014, p. 15-17 of Quaternary Structure: Product Aggregates and Measurement of Aggregates, https://www.fda.gov/media/85017/download; See also, Ratanji et al; Immunogenicity of therapeutic proteins: Influence of Aggregation. Journal of Immunotoxicology, 2014; 11 (2): 99-109; Wang et al; Immunogenicity of Protein Aggregates-Concerns and Realities, International Journal of Pharmaceutics, 2012, 431 (1-2): 1-11; and Moussa et al, Immunogenicity of Therapeutic Protein Aggregates, Journal of Pharmaceutical Sciences, 2016; 105(2): 417-430).

In addition, protein aggregation correlates with enzyme activity, as aggregation interferes with the ability of the enzyme to function and also can cause a reduction in the total yield of active enzyme. Protein aggregation causes challenges for manufacturing and development, delaying the time it takes to get therapeutics to patients and increasing cost. The recombinant crisantaspase of present disclosure demonstrates lower aggregation than other Erwinia chrysanthemi L-asparaginase recombinantly expressed in E. coli and Erwinia chrysanthemi-derived L-asparaginases. These aspects of the recombinant L-asparaginase make it an improvement over the art.

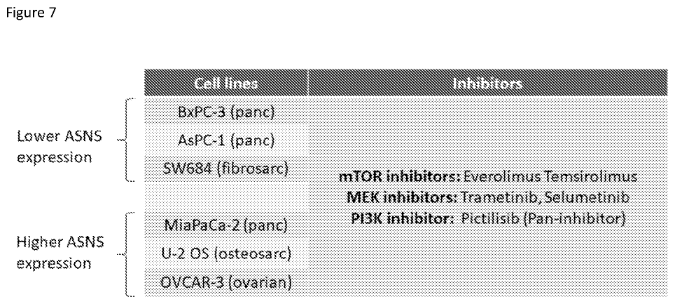

In some embodiments, a prognostic marker may be used to determine whether a patient would be successfully treated with an L-asparaginase therapy. As non-limiting examples, the prognostic marker can be a WnT pathway mutation, a MAPK pathway mutation, ASNS expression, or BCL2 expression. In some embodiments, measuring expression of ASNS or BCL2 may be used as a prognostic marker to determine whether a patient would be successful with treatment of a combination or monotherapy comprising an L-asparaginase (see FIGS. 7, 11, and 12).

The recombinant L-asparaginase of the present disclosure can have any combination of the properties in the above sections or any other properties described herein.

In some embodiments, combination therapies involving L-asparaginase result in synergistic results when compared to single agent testing with L-asparaginase or the selected combination therapy administered alone.

### E. Methods of Manufacturing a L-asparaginase

In some embodiments, the L-asparaginase disclosed herein is recombinantly produced in Pseudomonas fluorescens. In some embodiments, the Pseudomonas fluorescens is deficient in native L-asparaginase.

In some embodiments, the present disclosure provides methods for cytoplasmic production of a recombinant L-asparaginase in soluble form at high yields, wherein the recombinant protein is periplasmically produced at lower yields in its native host. In its native host, Erwinia chrysanthemi, L-asparaginase is produced in the periplasm. The present disclosure provides methods that allow production of high levels of soluble and/or active recombinant L-asparaginase in the cytoplasm of the host cell. In embodiments, methods provided herein yield high levels of soluble and/or active recombinant L-asparaginase in the cytoplasm of a Pseudomonadales, Pseudomonad, Pseudomonas, or Pseudomonas fluorescens host cell.

Methods that can be used for manufacturing a recombinant L-asparaginase are described for example in U.S. Pub. 2019/0127742, which is herein incorporated by reference in its entirety for all purposes and in particular for all teachings related to manufacturing methods for recombinant L-asparaginase. Further methods for isolating and/or developing L-asparaginase for use in the methods described herein include WO 2011/003886, WO 2018/234492, WO 2019/109018, WO 2021/078988, and PCT/US2021/032627, filed May 14, 2021, which are each herein incorporated by reference in its entirety for all purposes and in particular for all teachings related to manufacturing and development methods for L-asparaginase.

### IV. Combination Therapies for Use with a L-asparaginase

The present disclosure provides for methods of treating cancer in a patient comprising administering to the patient an effective amount of a protein or a conjugate of the protein having substantial L-asparagine aminohydrolase activity and another therapeutic.

In some embodiments, the present disclosure is directed to a method for treating cancer comprising co-administering to a patient in need of the treatment a therapeutically effective amount of a protein or a conjugate of the protein having substantial L-asparagine aminohydrolase activity and another therapeutic. In some embodiments, the conjugate of a protein having substantial L-asparagine aminohydrolase activity is administered with a combination of chemotherapy drugs, including, but not limited to glucocorticoids, corticosteroids, anticancer compounds or other agents, including, but not limited to methotrexate, dexamethasone, prednisone, prednisolone, vincristine, cyclophosphamide, and anthracycline. As an example, patients with ALL will be administered the conjugate of the present disclosure as a component of multi-agent chemotherapy during chemotherapy phases including induction, consolidation or intensification, and maintenance. The conjugate can be administered before, after, or simultaneously with other compounds as part of a multi-agent chemotherapy regimen.

In some embodiments, treatment with a L-asparaginase of the present disclosure is co-administered with a multi-agent chemotherapeutic regimen. In some embodiments, treatment with a L-asparaginase of the present disclosure is co-administered with one or more other chemotherapeutic agents as part of a multi-agent chemotherapeutic regimen. In some embodiments, treating patients with a L-asparaginase of the present disclosure in addition to other agents helps to ensure availability of an asparaginase for patients who have developed hypersensitivity to E. coli derived-asparaginase. In some embodiments, the L-asparaginase is part of a multi-agent regimen that includes a chemotherapeutic agent, an immunosuppressant, a corticosteroid, a glucocorticoid, a BCL-2 inhibitor, a BCL-XL inhibitor, an inhibitor of both BCL-XL and BCL-2, a glutaminase inhibitor, a topoisomerase inhibitor, an mTOR inhibitor, a BRAF inhibitor, a pan-RAF inhibitor, a VEGF inhibitor, a BTK inhibitor, a MEK inhibitor, a CD20 inhibitor, a checkpoint inhibitor, a MAPK pathway inhibitor, a RAF inhibitor, a RAS inhibitor, or a combination thereof. In some embodiments, the L-asparaginase is part of a multi-agent regimen that includes a chemotherapeutic agent, an immunosuppressant, a corticosteroid, a glucocorticoid, a BCL-2 inhibitor, a BCL-XL inhibitor, an inhibitor of both BCL-XL and BCL-2, a glutaminase inhibitor, a topoisomerase inhibitor, an mTOR inhibitor, a BRAF inhibitor, a pan-RAF inhibitor, a VEGF inhibitor, a BTK inhibitor, a MEK inhibitor, a CD20 inhibitor, a checkpoint inhibitor, or a combination thereof. Examples of agents that can be part of a multi-agent chemotherapeutic regimen with a L-asparaginase of the present disclosure include, but are not limited to: cytarabine, vincristine, daunorubicin, methotrexate, leuvocorin, doxorubicin, anthracycline, corticosteroids and glucocortiods (including but not limited to prednisone, prednisolone, and/or dexamethasone), cyclophosphamide, 6-mercaptopurine, venetoclax, etoposide, regorafenib, encorafenib, lonsurf, vemurafenib, avastin, blinatumomab, gemcitabine, binimetanib, and cobimetinib. In some embodiments, the multi-agent chemotherapeutic regimen includes the L-asparaginase and cytarabine, vincristine, daunorubicin, methotrexate, leuvocorin, doxorubicin, anthracycline, corticosteroids and glucocortiods (including but not limited to prednisone, prednisolone, and/or dexamethasone), cyclophosphamide, 6-mercaptopurine, venetoclax, etoposide, or a combination thereof. In some embodiments, the multi-agent chemotherapeutic regimen is the L-asparaginase and one additional chemotherapeutic agent. In some embodiments, the multi-agent chemotherapeutic regimen is the L-asparaginase and two or more additional chemotherapeutic agents.

As an example, patients with ALL will be co-administered the L-asparaginase of the present disclosure along with a multi-agent chemotherapy during 3 chemotherapy phases including induction, consolidation or intensification, and maintenance. In a specific example, the L-asparaginase of the present disclosure is co-administered with an asparagine synthetase inhibitor (e.g., such as set forth in WO 2007/103290, which is herein incorporated by reference in its entirety). In another specific example, the L-asparaginase of the present disclosure is not co-administered with an asparagine synthetase inhibitor, but is co-administered with other chemotherapy drugs. In another specific example, the L-asparaginase of the present disclosure is co-administered with an asparagine synthetase inhibitor and other chemotherapy drugs. The L-asparaginase of the present disclosure can be co-administered before, after, or simultaneously with other compounds as part of a multi-agent chemotherapy regimen. In a particular embodiment, the L-asparaginase of the present disclosure comprises a protein recombinantly produced in Pseudomonas fluorescens, and more specifically, the recombinant L-asparaginase comprising the sequence of SEQ ID NO:1.

If a patient treated with E. coli-derived L-asparaginase has a relapse, subsequent treatment with E. coli preparations could lead to a “vaccination” effect, whereby the E. coli preparation has increased immunogenicity during the subsequent administrations. In one embodiment, the conjugate of the present disclosure can be used in a method of treating patients who were previously treated with other asparaginase preparations, in particular those who were previously treated with E. coli-derived asparaginases.

In some embodiments, the uses and methods of treatment of the present disclosure comprise administering an L-asparaginase conjugate having properties or combinations of properties described herein above (e.g., in the section entitled L-asparaginase PEG conjugates or pasylated L-asparaginase) or herein below.

In some embodiments, the present disclosure provides for methods of treating cancer in a patient comprising administering to the patient an effective amount of a protein or a conjugate of the protein having substantial L-asparagine aminohydrolase activity and a BCL-XL inhibitor. In some embodiments, the present disclosure provides for methods of treating cancer in a patient comprising administering to the patient an effective amount of a protein or a conjugate of the protein having substantial L-asparagine aminohydrolase activity and a BCL-2 inhibitor. In some embodiments, the present disclosure provides for methods of treating cancer in a patient comprising administering to the patient an effective amount of a protein or a conjugate of the protein having substantial L-asparagine aminohydrolase activity and an inhibitor of both BCL-XL and BCL-2. In some embodiments, the present disclosure provides for methods of treating cancer in a patient comprising administering to the patient an effective amount of a protein or a conjugate of the protein having substantial L-asparagine aminohydrolase activity and a mTOR inhibitor. In some embodiments, the present disclosure provides for methods of treating cancer in a patient comprising administering to the patient an effective amount of a protein or a conjugate of the protein having substantial L-asparagine aminohydrolase activity and a CD20 inhibitor. In some embodiments, the present disclosure provides for methods of treating cancer in a patient comprising administering to the patient an effective amount of a protein or a conjugate of the protein having substantial L-asparagine aminohydrolase activity and a BTK inhibitor. In some embodiments, the present disclosure provides a method of treating cancer in a patient including administering to the patient an effective amount of a protein having substantial L-asparagine aminohydrolase activity and a BRAF inhibitor. In some embodiments, the present disclosure provides a method of treating cancer in a patient including administering to the patient an effective amount of a protein having substantial L-asparagine aminohydrolase activity and a pan-RAF inhibitor. In some embodiments, the present disclosure provides a method of treating cancer in a patient including administering to the patient an effective amount of a protein having substantial L-asparagine aminohydrolase activity and a VEGF inhibitor. In some embodiments, the present disclosure provides a method of treating cancer in a patient including administering to the patient an effective amount of a protein having substantial L-asparagine aminohydrolase activity and a MEK inhibitor. In some embodiments, the present disclosure provides a method of treating cancer in a patient including administering to the patient an effective amount of a protein having substantial L-asparagine aminohydrolase activity and a checkpoint inhibitor.

### A. L-asparaginase and BCL-XL Inhibitors

In some embodiments, the uses and methods of treatment of the present disclosure comprise administering an L-asparaginase with a B-cell lymphoma-extra large (BCL-XL) inhibitor. BCL-XL is an anti-apoptotic transmembrane protein located in the mitochondria and prevents the release of mitochondrial contents such as cytochrome c. BCL-XL is also known to inhibit apoptosis through inhibition of Bax. In some embodiments, BCL-XL inhibitors act to reduce overexpression of BCL-XL and reduce resistance to chemotherapeutics.

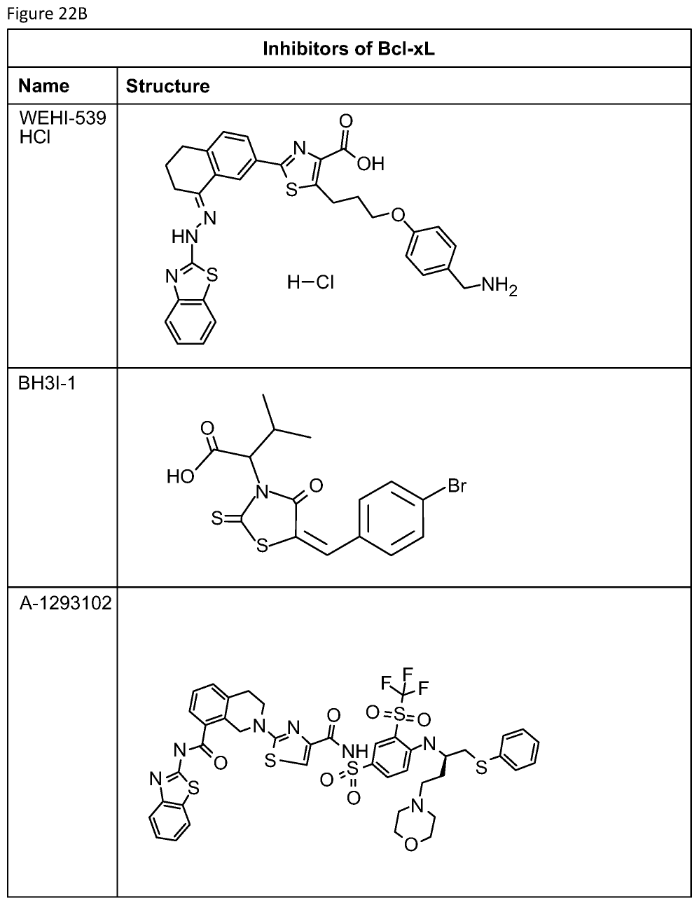

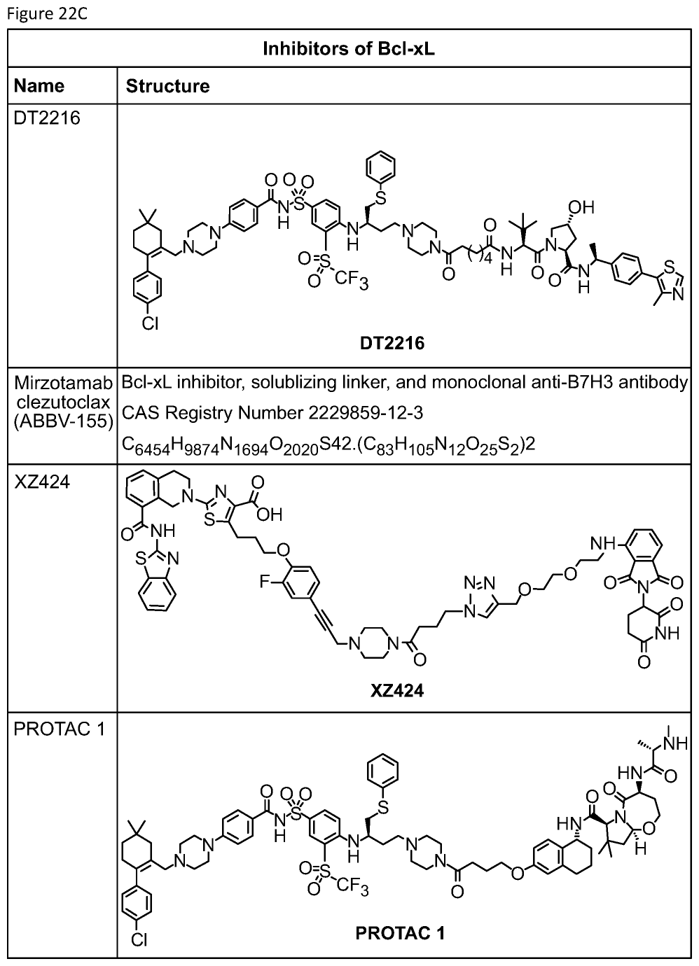

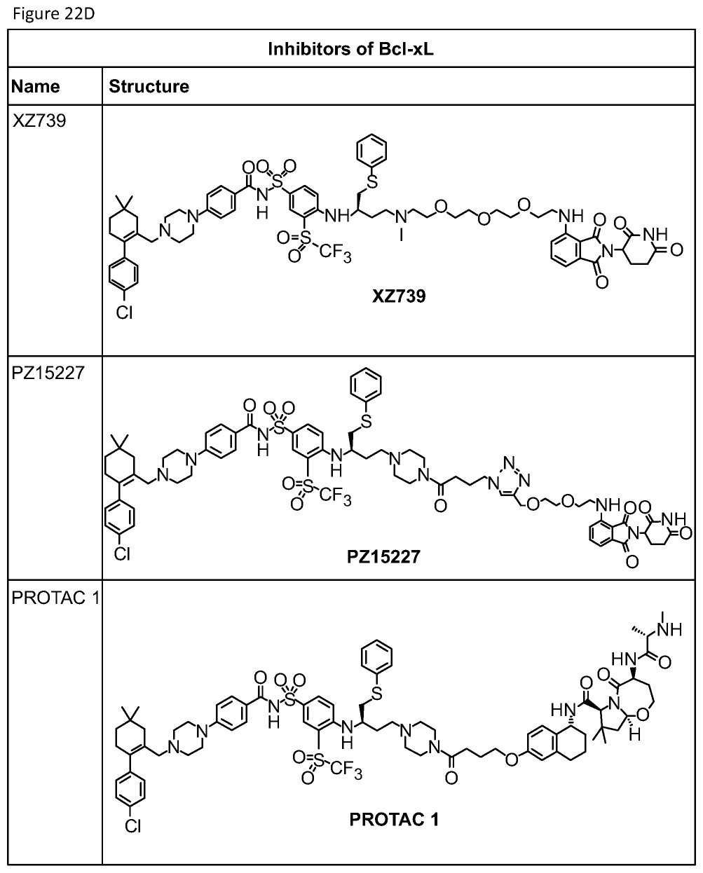

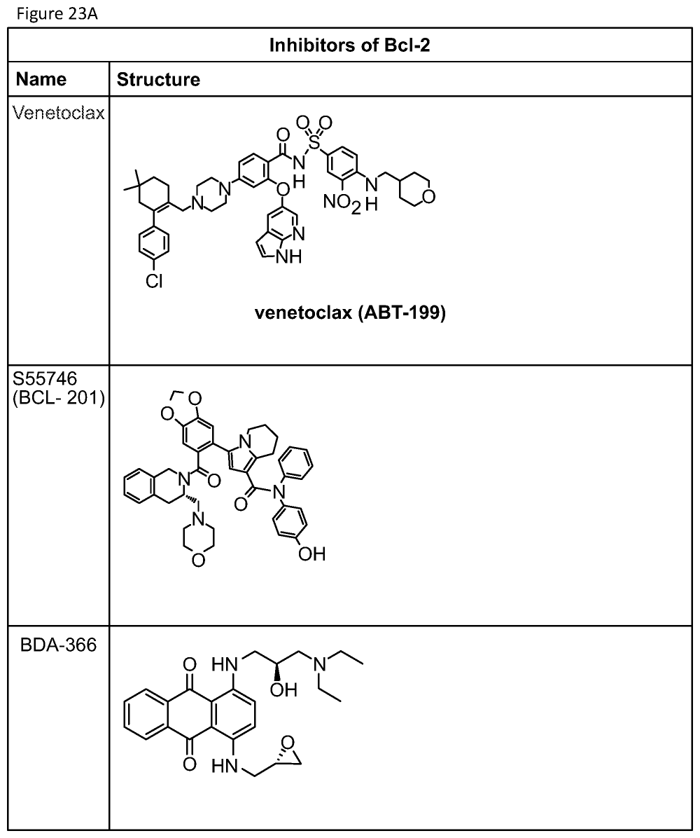

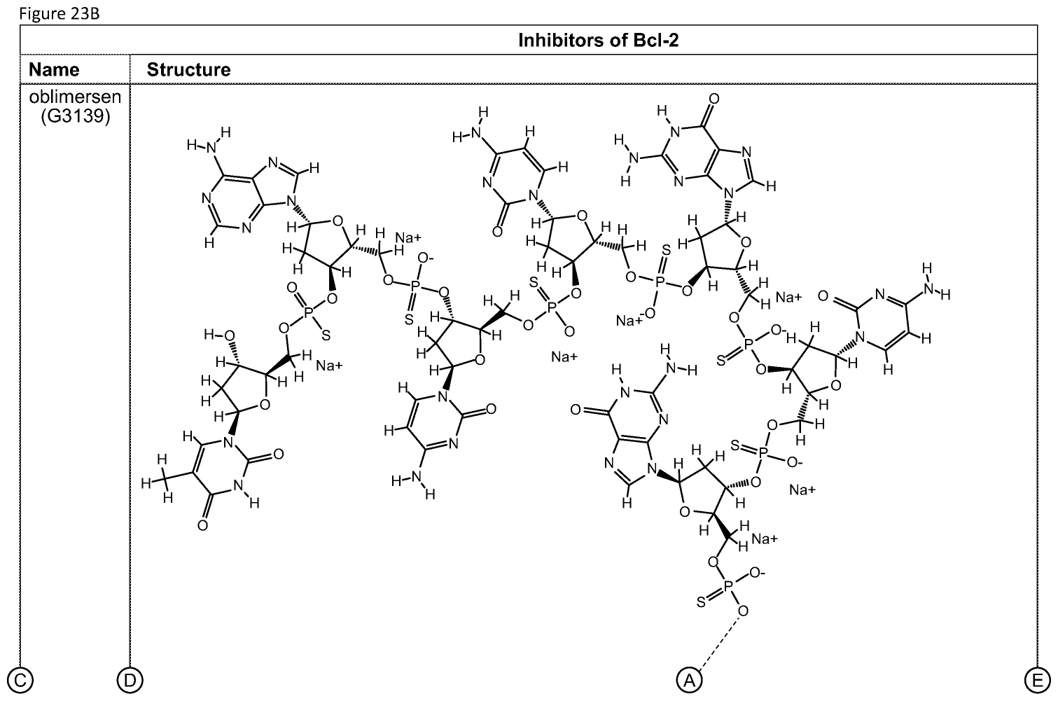

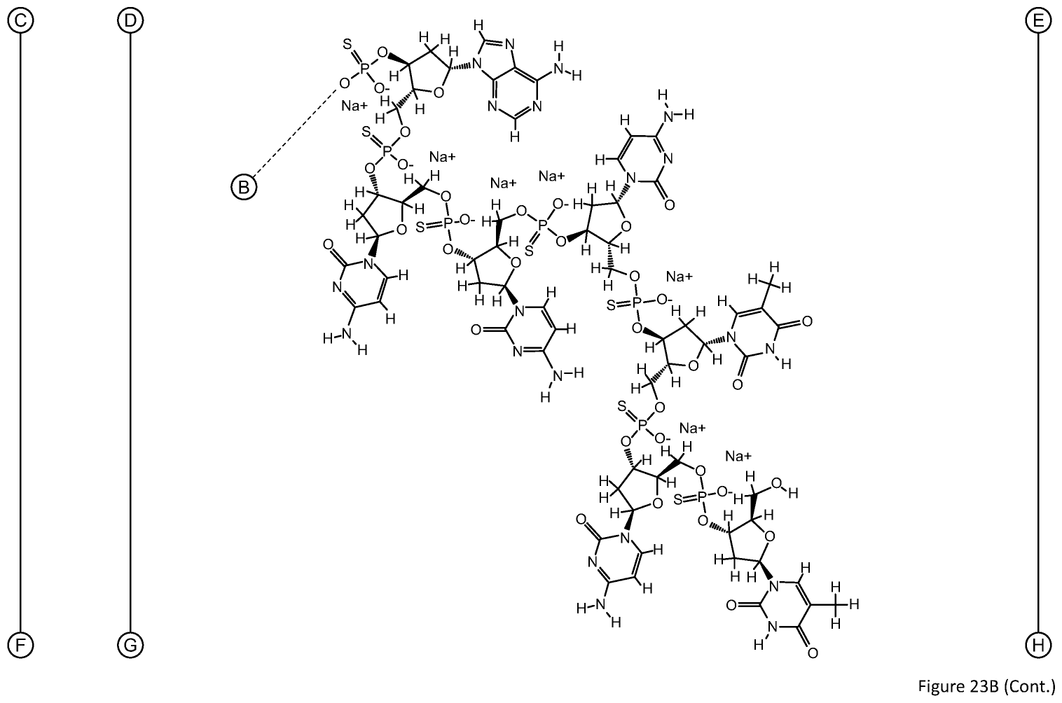

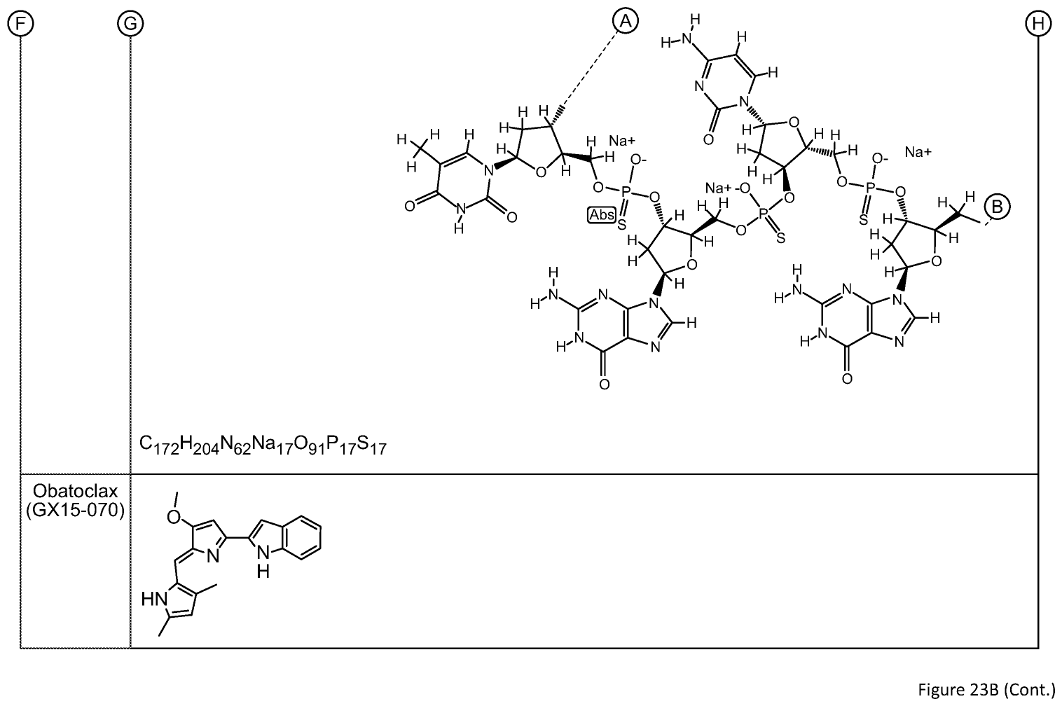

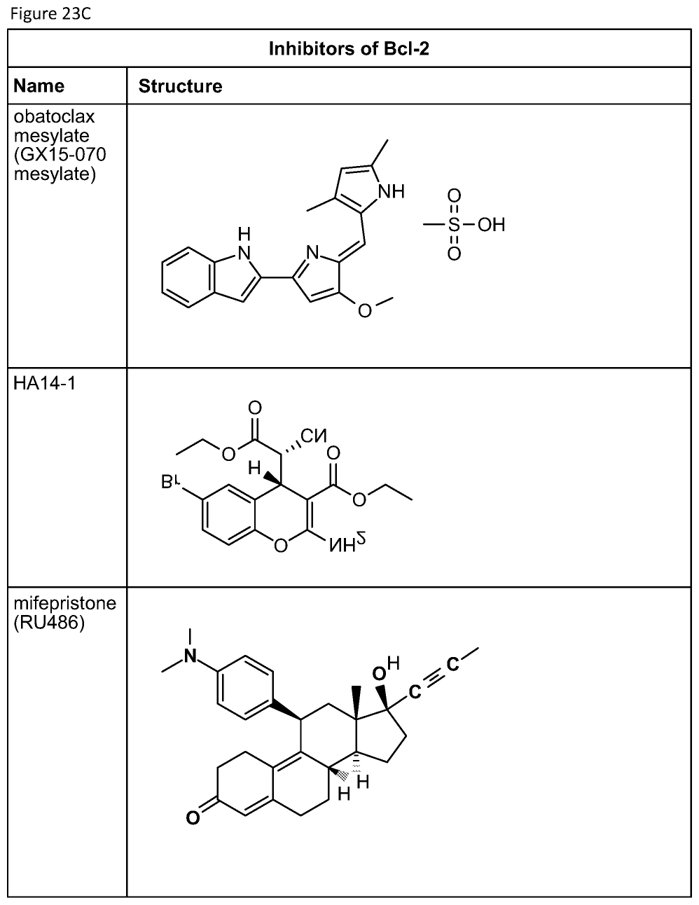

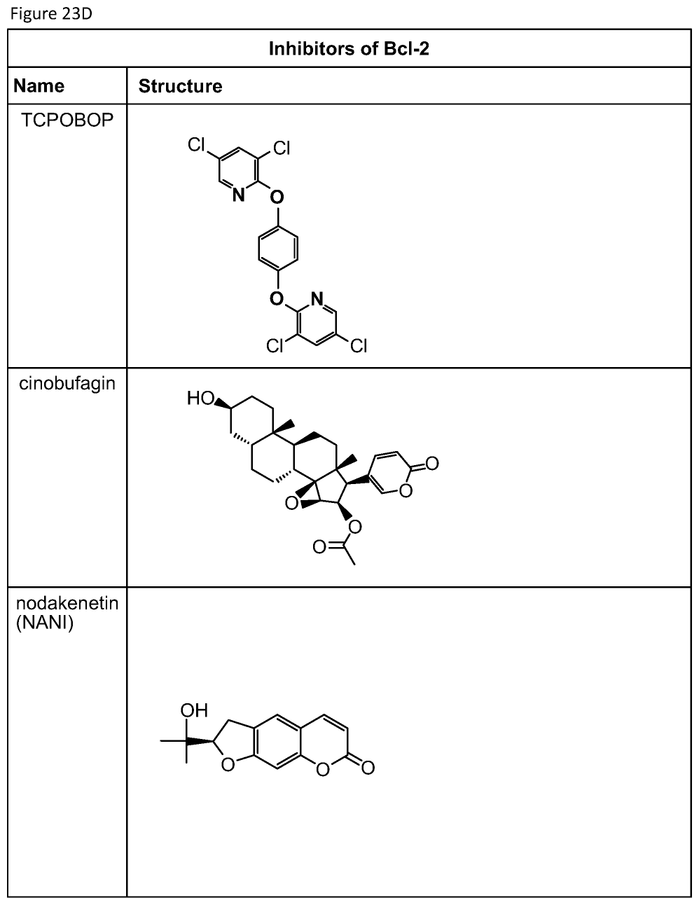

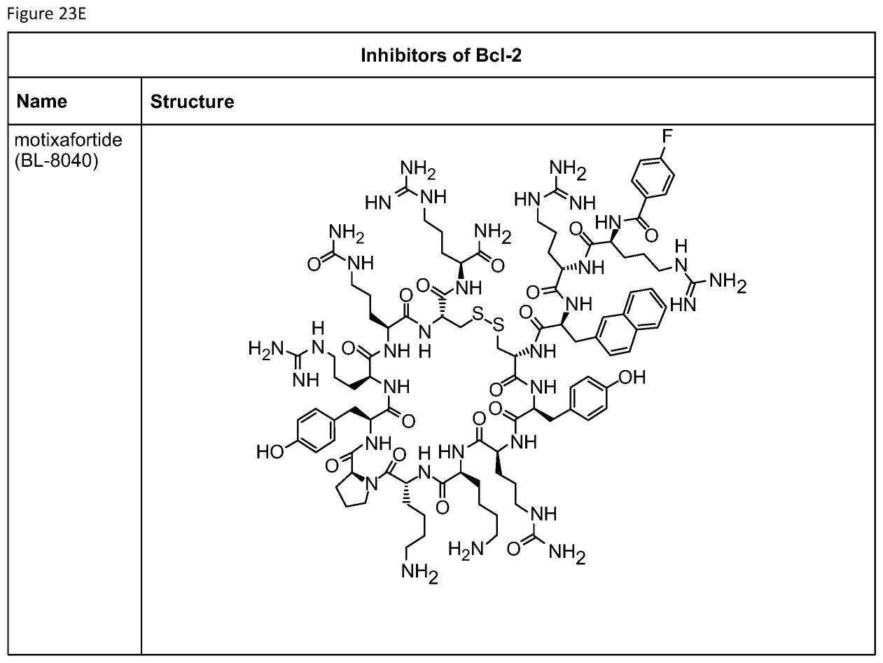

BCL-XL inhibitors that can be used in the present disclosure comprise a small molecule that inhibits BCL-XL, an antibody that inhibits BCL-XL, an antibody-drug conjugate with a BCL-XL payload, a dendrimer with a BCL-XL payload, a prodrug that targets BCL-XL, a proteolysis targeting chimera (PROTAC) that targets BCL-XL, or any other modality that targets BCL-XL. Examples of BCL-XL inhibitors include but are not limited to: A-1155463, A-1331852, WEHI-539, WEHI-539 HCl, BH31-1, A-1293102, DT2216, XZ424, XZ739, PZ15227, PROTAC 1, and ABBV-155. In some embodiments, the BCL-XL inhibitor is PROTAC 1 (as disclosed in Zhang et al., PROTACs are effective in addressing the platelet toxicity associated with BCL-XL inhibitors. Explor Target Antitumor Ther. 2020; 1:259-272). Examples of BCL-XL inhibitors include but are not limited to those found in FIG. 22A-22D. Additional examples of BCL-XL inhibitors may be found in Zhang et al., PROTACs are effective in addressing the platelet toxicity associated with BCL-XL inhibitors. Explor Target Antitumor Ther. 2020; 1:259-272, which is hereby incorporated by reference for its disclosure of PROTAC1 and other examples of BCL-XL inhibitors.

In some embodiments, combination therapies involving an L-asparaginase and BCL-XL result in a synergistic effect when administered to treat cancer.

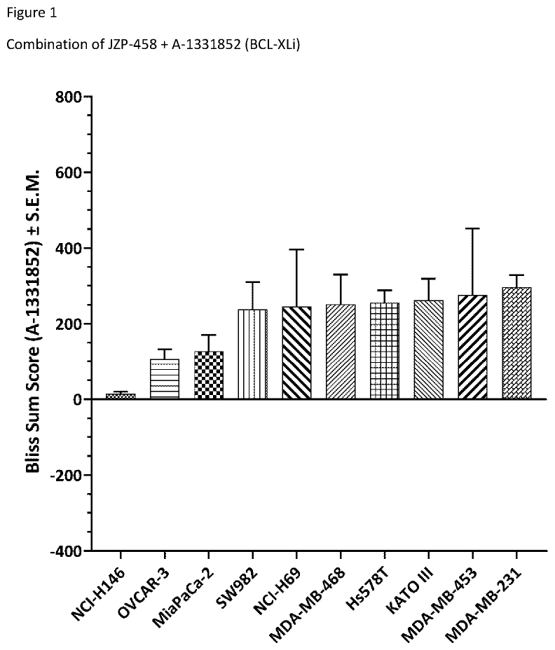

Data showing combination therapies involving an L-asparaginase and BCL-XL inhibitors can be found in FIGS. 1, 5, and 9.

In some embodiments, described herein are combination therapies for lung cancer, wherein the combination therapies comprise the use of L-asparaginase (e.g., pasylated or unfunctionalized L-asparaginase with homology to SEQ ID NO:1) and BCL-XL inhibitors. In further embodiments, the combination shows a synergistic effect against lung cancer. In further embodiments, the combination therapy with the synergistic effect against lung cancer is a combination comprising L-asparaginase and A1331852 or A1155463. In yet further embodiments, the synergy is seen when the combination therapy includes a recombinant L-asparaginase, including the L-asparaginase referred to as JZP-458 and/or Erwinase. In still further embodiments, the synergistic effect presents as a BLISS score from a lung cancer cell line (see for example Figure land FIG. 5). In further embodiments, the synergy of the L-asparaginase and BCL-XL inhibitor is shown in assays involving the lung cancer cell line NCI-H146 or the lung cancer cell line NCI-H69 (see for example FIG. 1 and FIG. 5).

In some embodiments, described herein are combination therapies for small cell lung cancer (SCLC), wherein the combination therapies comprise the use of L-asparaginase and BCL-XL inhibitors. In further embodiments, the combination shows a synergistic effect against SCLC. In further embodiments, the combination therapy with the synergistic effect against SCLC is a combination comprising L-asparaginase and A1331852 or A1155463. In yet further embodiments, the synergy is seen when the combination therapy includes a recombinant L-asparaginase, including the L-asparaginase referred to as JZP-458 and/or Erwinase. In still further embodiments, the synergistic effect presents as a BLISS score from an SCLC cell line (see for example Figure land FIG. 5). In further embodiments, the synergy of the L-asparaginase and BCL-XL inhibitor is shown in assays involving the SCLC cell line NCI-H146 or the SCLC cell line NCI-H69 (see for example FIG. 1 and FIG. 5).

In some embodiments, described herein are combination therapies for pancreatic cancer, wherein the combination therapies comprise the use of L-asparaginase and BCL-XL inhibitors. In further embodiments, the combination shows a synergistic effect against pancreatic cancer. In further embodiments, the combination therapy with the synergistic effect against pancreatic cancer is a combination comprising L-asparaginase and A1331852 or A1155463. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a pancreatic cell line (see for example FIG. 1 and FIG. 5). In further embodiments, the synergy of the L-asparaginase and BCL-XL inhibitor is shown in assays involving the pancreatic cancer cell line MiaPaCa-2 (see for example FIG. 1 and FIG. 5).

In some embodiments, described herein are combination therapies for breast cancer, wherein the combination therapies comprise the use of L-asparaginase and BCL-XL inhibitors. In further embodiments, the combination shows a synergistic effect against breast cancer. In further embodiments, the combination therapy with the synergistic effect against pancreatic cancer is a combination comprising L-asparaginase and A1331852 or A1155463. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a breast cancer cell line (see for example FIG. 1 and FIG. 5). In further embodiments, the synergy of the L-asparaginase and BCL-XL inhibitor is shown in assays involving the breast cancer cell lines MDA-MB-231, MDA-MB-453, Hs578T, or MDA-MB-468 (see for example FIG. 1 and FIG. 5). In some embodiments, the breast cancer is triple negative breast cancer (TNBC).

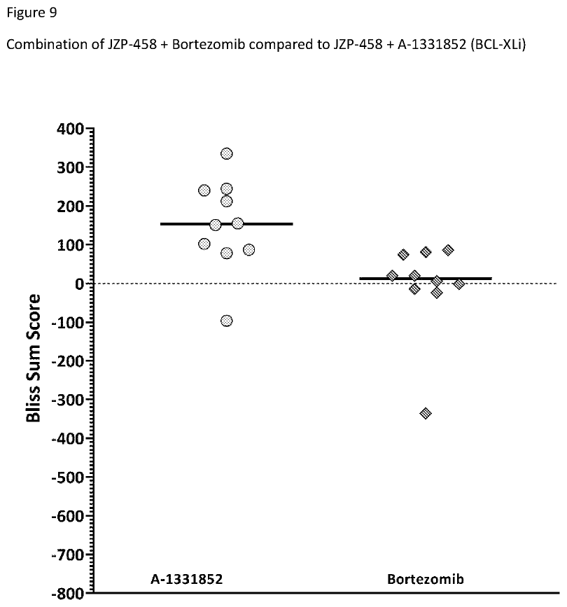

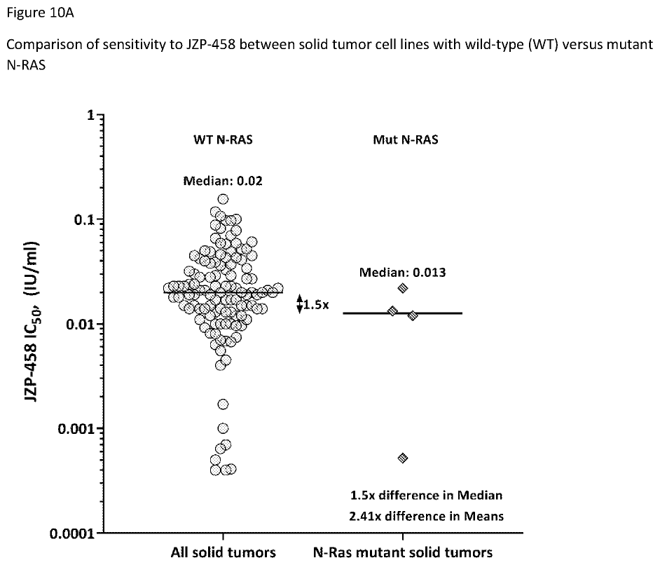

In some embodiments, described herein are combination therapies for ovarian cancer, wherein the combination therapies comprise the use of L-asparaginase and BCL-XL inhibitors. In further embodiments, the combination shows a synergistic effect against ovarian cancer. In further embodiments, the combination therapy with the synergistic effect against ovarian cancer is a combination comprising L-asparaginase and A1331852 or A1155463. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from an ovarian cell line (see for example FIG. 1, FIG. 5, and FIG. 9). In further embodiments, the synergy of the L-asparaginase and BCL-XL inhibitor is shown in assays involving the ovarian cancer cell line OVCAR-3 (see for example FIG. 1, FIG. 5, and FIG. 9).

In some embodiments, described herein are combination therapies for sarcoma, wherein the combination therapies comprise the use of L-asparaginase and BCL-XL inhibitors. In further embodiments, the combination shows a synergistic effect against sarcoma. In further embodiments, the combination therapy with the synergistic effect against sarcoma is a combination comprising L-asparaginase and A1331852 or A1155463. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a sarcoma cell line (see for example FIG. 1, FIG. 5, and FIG. 9. In further embodiments, the synergy of the L-asparaginase and BCL-XL inhibitor is shown in assays involving the sarcoma cell line SW982 (see for example FIG. 1 and FIG. 5).

In some embodiments, described herein are combination therapies for gastric cancer, wherein the combination therapies comprise the use of L-asparaginase and BCL-XL inhibitors. In further embodiments, the combination shows a synergistic effect against gastric cancer. In further embodiments, the combination therapy with the synergistic effect against gastric cancer is a combination comprising L-asparaginase and A1331852 or A1155463. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a gastric cancer cell line (see for example FIG. 1 and FIG. 5). In further embodiments, the synergy of the L-asparaginase and BCL-XL inhibitor is shown in assays involving the gastric cancer cell line KATO III (see for example FIG. 1, FIG. 5, FIG. 9.

As will be appreciated and as is discussed in further detail herein, any of the above indications treated with any of the L-asparaginase and BCL-XL inhibitors described herein may be treated by treating with L-asparaginase prior to treating with a BCL-XL inhibitor, by treating with L-asparaginase subsequent to treating with a BCL-XL inhibitor, or by treating with L-asparaginase simultaneously with a BCL-XL inhibitor. Administration may be provided by separate compositions, administration at different times in separate compositions, or administration in a composition in which two or more active pharmaceutical ingredients are present. Simultaneous administration in separate compositions and administration in a composition in which both L-asparaginase and a BCL-XL inhibitor are present is also described herein. Depending on how each one of the active pharmaceutical ingredients is delivered the frequency of dosing may vary. In some embodiments, each or both of the L-asparaginase and BCL-XL inhibitor can be given every one day, two days, three days, four days, five days, six days, one week, two weeks, three weeks, 1 month, 2 months, or three months. In some embodiments, the BCL-XL inhibitor is administered following administration with L-asparaginase. In some embodiments, the BCL-XL inhibitor is administered before administration with L-asparaginase. In some embodiments, there are at least 3-6 h between the administration of the L-asparaginase and the BCL-XL inhibitor. In some embodiments there is one, two, three, four, five, six, seven, eight, nine, ten, eleven, twelve hours between the administration of the L-asparaginase and the BCL-XL inhibitor. In some embodiments there are about 24 hours between the administration of the L-asparaginase and the BCL-XL inhibitor. In some embodiments there are about 48 hours between the administration of the L-asparaginase and the BCL-XL inhibitor. Administration of the L-asparaginase and the BCL-XL inhibitor can be by many different routes, including, but not limited to: intravenous, intraperitoneal, subcutaneous, intramuscular, oral, topical (transdermal), or transmucosal administration. In some embodiments, the route of administration for L-asparaginase and the BCL-XL inhibitor will be the same. In some embodiments, the route of administration for L-asparaginase and the BCL-XL inhibitor will be different. In some embodiments, the route of administration for the BCL-XL inhibitor will be intravenous administration. In some embodiments, the BCL-XL inhibitor will be administered every two weeks. In some embodiments, the BCL-XL inhibitor will be administered every three weeks. In some embodiments, the BCL-XL inhibitor will be administered every four weeks. In some embodiments, the route of administration for the L-asparaginase will be intravenous administration. In some embodiments, the route of administration for the L-asparaginase will be intramuscular administration.

### B. L-asparaginase and BCL-2 Inhibitors

In some embodiments, the uses and methods of treatment of the present disclosure comprise administering an L-asparaginase with a B-cell lymphoma 2 (BCL-2) inhibitor. BCL-2 is an apoptosis regulator protein encoded by the BCL2 gene that exhibits anti-apoptotic activity and affects mitochondrial regulation.

BCL-2 inhibitors that may be used in the present disclosure include, but are not limited to a small molecule that inhibits BCL-2, an antibody that inhibits BCL-2, an antibody-drug conjugate with a BCL-2 payload, a dendrimer with a BCL-2 payload, a prodrug that targets BCL-2, a proteolysis targeting chimera (PROTAC) that targets BCL-2, or any other modality that targets BCL-2. Examples of BCL-2 inhibitors, include but are not limited to: venetoclax (ABT-199), S55746, BDA-366, oblimersen (G3139), obatoclax, obatoclax mesylate (GX15-070), HA14-1, mifepristone (RU486), TCPOBOP, cinobufagin, nodakenetin (NANI), and motixafortide (BL-8040). Additional examples of BCL-2 inhibitors may be found in Zhang et al., PROTACs are effective in addressing the platelet toxicity associated with BCL-XL inhibitors. Explor Target Antitumor Ther. 2020; 1:259-272, which is hereby incorporated by reference for its disclosure of BCL-2 inhibitors.

In some embodiments, combination therapies involving an L-asparaginase and BCL-2 result in a synergistic effect when administered to treat cancer.

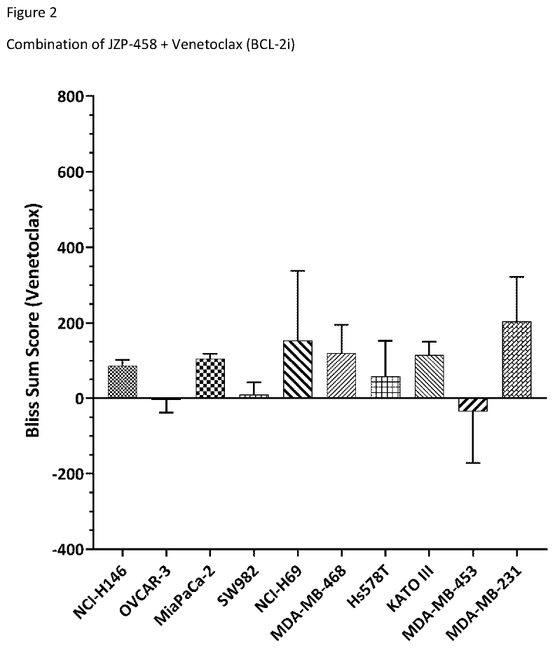

Data showing combination therapies involving an L-asparaginase and BCL-2 inhibitors can be found in FIGS. 2, 13, 18, and 19.

In some embodiments, described herein are combination therapies for lung cancer, wherein the combination therapies comprise the use of L-asparaginase and BCL-2 inhibitors. In further embodiments, the combination shows a synergistic effect against lung cancer. In further embodiments, the combination therapy with the synergistic effect against lung cancer is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a lung cancer cell line (see for example FIG. 2). In further embodiments, the synergy of the L-asparaginase and a BCL-2 inhibitor is shown in assays involving the lung cancer cell line NCI-H146 or the lung cancer cell line NCI-H69 (see for example FIG. 2).

In some embodiments, described herein are combination therapies for small cell lung cancer (SCLC), wherein the combination therapies comprise the use of L-asparaginase and BCL-2 inhibitors. In further embodiments, the combination shows a synergistic effect against SCLC. In further embodiments, the combination therapy with the synergistic effect against SCLC is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from an SCLC cell line (see for example FIG. 2). In further embodiments, the synergy of the L-asparaginase and a BCL-2 inhibitor is shown in assays involving the SCLC cell line NCI-H146 or the SCLC cell line NCI-H69 (see for example FIG. 2).

In some embodiments, described herein are combination therapies for pancreatic cancer, wherein the combination therapies comprise the use of L-asparaginase and BCL-2 inhibitors. In further embodiments, the combination shows a synergistic effect against pancreatic cancer. In further embodiments, the combination therapy with the synergistic effect against pancreatic cancer is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a pancreatic cell line (see for example FIG. 2). In further embodiments, the synergy of the L-asparaginase and a BCL-2 inhibitor is shown in assays involving the pancreatic cancer cell line MiaPaCa-2 (see for example FIG. 2).

In some embodiments, described herein are combination therapies for breast cancer, wherein the combination therapies comprise the use of L-asparaginase and BCL-2 inhibitors. In further embodiments, the combination shows a synergistic effect against breast cancer. In further embodiments, the combination therapy with the synergistic effect against pancreatic cancer is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a breast cancer cell line (see for example FIG. 2). In further embodiments, the synergy of the L-asparaginase and a BCL-2 inhibitor is shown in assays involving the breast cancer cell lines MDA-MB-231, Hs578T, or MDA-MB-468 (see for example FIG. 2).

In some embodiments, described herein are combination therapies for ovarian cancer, wherein the combination therapies comprise the use of L-asparaginase and BCL-2 inhibitors. In further embodiments, the combination shows a synergistic effect against ovarian cancer. In further embodiments, the combination therapy with the synergistic effect against ovarian cancer is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from an ovarian cell line (see for example FIG. 2). In further embodiments, the synergy of the L-asparaginase and a BCL-2 inhibitor is shown in assays involving the ovarian cancer cell line OVCAR-3 (see for example FIG. 2).

In some embodiments, described herein are combination therapies for sarcoma, wherein the combination therapies comprise the use of L-asparaginase and BCL-2 inhibitors. In further embodiments, the combination shows a synergistic effect against sarcoma. In further embodiments, the combination therapy with the synergistic effect against sarcoma is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a sarcoma cell line (see for example FIG. 2). In further embodiments, the synergy of the L-asparaginase and a BCL-2 inhibitor is shown in assays involving the sarcoma cell line SW982 (see for example FIG. 2).

In some embodiments, described herein are combination therapies for gastric cancer, wherein the combination therapies comprise the use of L-asparaginase and BCL-2 inhibitors. In further embodiments, the combination shows a synergistic effect against gastric cancer. In further embodiments, the combination therapy with the synergistic effect against gastric cancer is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a gastric cancer cell line (see for example FIG. 2). In further embodiments, the synergy of the L-asparaginase and a BCL-2 inhibitor is shown in assays involving the gastric cancer cell line KATO III (see for example FIG. 2.

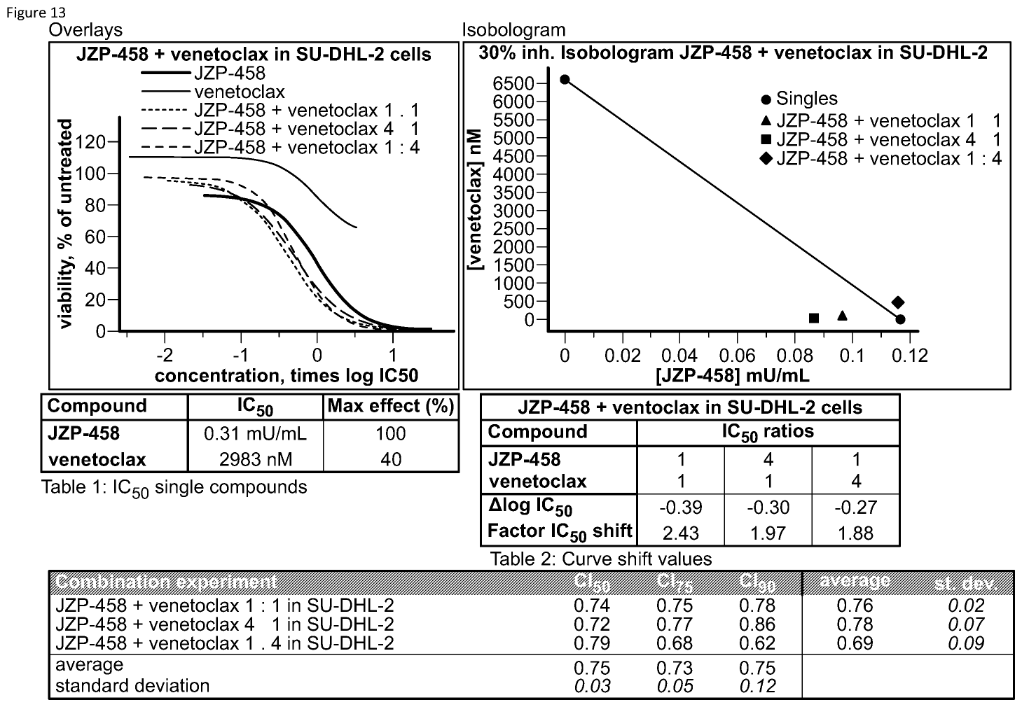

In some embodiments, described herein are combination therapies for lymphoma, wherein the combination therapies comprise the use of L-asparaginase and BCL-2 inhibitors. In further embodiments, the combination shows a synergistic effect against lymphoma. In further embodiments, the combination therapy with the synergistic effect against lymphoma is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a lymphoma cell line (see for example FIG. 13). In further embodiments, the synergy of the L-asparaginase and a BCL-2 inhibitor is shown in assays involving lymphoma cell lines (see for example FIG. 13).

In some embodiments, described herein are combination therapies for diffuse large b cell lymphoma (DLBCL), wherein the combination therapies comprise the use of L-asparaginase and BCL-2 inhibitors. In further embodiments, the combination shows a synergistic effect against DLBCL. In further embodiments, the combination therapy with the synergistic effect against DLBCL is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a DLBCL cell line. In further embodiments, the synergy of the L-asparaginase and a BCL-2 inhibitor is shown in assays involving DLBCL cell lines.

As will be appreciated and as is discussed in further detail herein, any of the above indications treated with any of the L-asparaginase and BCL-2 inhibitors described herein may be treated by treating with L-asparaginase prior to treating with a BCL-2 inhibitor, by treating with L-asparaginase subsequent to treating with a BCL-2 inhibitor, or by treating with L-asparaginase simultaneously with a BCL-2 inhibitor. Administration may be provided by separate compositions, administration at different times in separate compositions, or administration in a composition in which two or more active pharmaceutical ingredients are present. Simultaneous administration in separate compositions and administration in a composition in which both L-asparaginase and a BCL-2 inhibitor are present is also described herein. Depending on how each one of the active pharmaceutical ingredients is delivered the frequency of dosing may vary. In some embodiments, each or both of the L-asparaginase and BCL-2 inhibitor can be given every one day, two days, three days, four days, five days, six days, one week, two weeks, three weeks, 1 month, 2 months, or three months. In some embodiments, the BCL-2 inhibitor is administered following administration with L-asparaginase. In some embodiments, the BCL-2 inhibitor is administered before administration with L-asparaginase. In some embodiments, there are at least 3-6 h between the administration of the L-asparaginase and the BCL-2 inhibitor. In some embodiments there is one, two, three, four, five, six, seven, eight, nine, ten, eleven, twelve hours between the administration of the L-asparaginase and the BCL-2 inhibitor. In some embodiments there are about 24 hours between the administration of the L-asparaginase and the BCL-2 inhibitor. In some embodiments there are about 48 hours between the administration of the L-asparaginase and the BCL-2 inhibitor. Administration of the L-asparaginase and the BCL-2 inhibitor can be by many different routes, including, but not limited to: intravenous, intraperitoneal, subcutaneous, intramuscular, oral, topical (transdermal), or transmucosal administration. In some embodiments, the route of administration for L-asparaginase and the BCL-2 inhibitor will be the same. In some embodiments, the route of administration for L-asparaginase and the BCL-2 inhibitor will be different. In some embodiments, the route of administration for the BCL-2 inhibitor will be intravenous administration. In some embodiments, the BCL-2 inhibitor will be administered every two weeks. In some embodiments, the BCL-2 inhibitor will be administered every three weeks. In some embodiments, the BCL-2 inhibitor will be administered every four weeks. In some embodiments, the route of administration for the L-asparaginase will be intravenous administration. In some embodiments, the route of administration for the L-asparaginase will be intramuscular administration.

C. L-asparaginase with Inhibitors of Both BCL-2 and BCL-XL

In some embodiments, the uses and methods of treatment of the present disclosure comprise administering an L-asparaginase with an inhibitor of both B-cell lymphoma-extra large (BCL-XL) and B-cell lymphoma 2 (BCL-2). In some embodiments, an inhibitor of both BCL-XL and BCL-2 can be an inhibitor specific to these two targets. In some embodiments, an inhibitor of both BCL-XL and BCL-2 can inhibit these targets along with additional targets. In some embodiments, one active pharmaceutical ingredient or active pharmaceutical agent is an inhibitor of both BCL-XL and BCL-2. In some embodiments, two or more active pharmaceutical ingredients or active pharmaceutical agents are inhibitors of both BCL-XL and BCL-2.

Examples of inhibitors of both BCL-XL and BCL-2 that can be used in the present disclosure include, but are not limited to: a small molecule that inhibits both BCL-XL and BCL-2, an antibody that inhibits both BCL-XL and BCL-2, an antibody-drug conjugate with both a BCL-XL and BCL-2 payload, a dendrimer with both a BCL-XL and BCL-2 payload, a prodrug that targets both BCL-XL and BCL-2, a proteolysis targeting chimera (PROTAC) that targets both BCL-XL and BCL-2, or any other modality that targets both BCL-XL and BCL-2. Examples of both BCL-XL and BCL-2 inhibitors include but are not limited to: navitoclax (ABT-263), ABT-737, sabutoclax, gossypol, (R)-(−)-gossypol acetic acid, TW-37, gambogic acid, 2-Methoxy-antimycin A, berberine chloride (NSC 646666), berberine chloride hydrate, APG-1252, AZD-0466, BM-1197, AZD4320 (dendrimer conjugate with AZD4320 as a payload), and pelcitoclax (APG-1252). Additional examples of inhibitors of both BCL-XL and BCL-2 may be found in Zhang et al., PROTACs are effective in addressing the platelet toxicity associated with BCL-XL inhibitors. Explor Target Antitumor Ther. 2020; 1:259-272, which is hereby incorporated by reference for its disclosure of BCL-2 inhibitors.

In some embodiments, combination therapies involving an L-asparaginase and an inhibitor of both BCL-XL and BCL-2 result in a synergistic effect when administered to treat cancer.

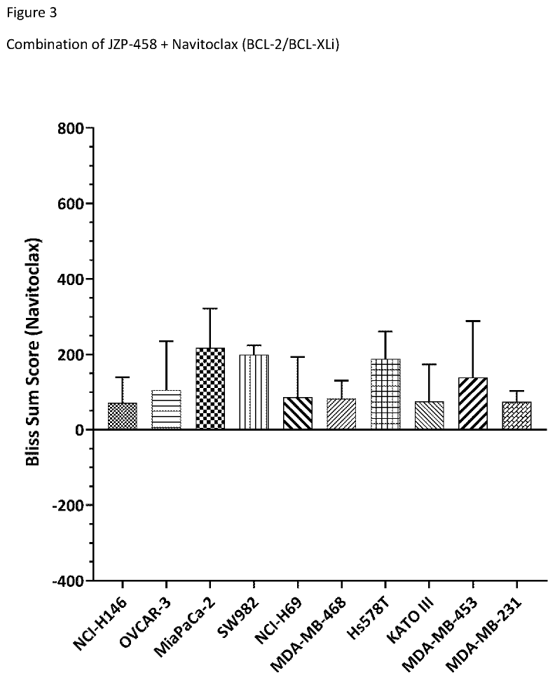

Examples of combination therapies involving an L-asparaginase and an inhibitor of both BCL-XL and BCL-2 inhibitors can be found in FIG. 3.

In some embodiments, described herein are combination therapies for lung cancer, wherein the combination therapies comprise the use of L-asparaginase and an inhibitor of both BCL-XL and BCL-2. In further embodiments, the combination shows a synergistic effect against lung cancer. In further embodiments, the combination therapy with the synergistic effect against lung cancer is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a lung cancer cell line (see for example FIG. 3). In further embodiments, the synergy of the L-asparaginase and an inhibitor of both BCL-XL and BCL-2 is shown in assays involving the lung cancer cell line NCI-H146 (see for example FIG. 3) or the lung cancer cell line NCI-H69.

In some embodiments, described herein are combination therapies for small cell lung cancer (SCLC), wherein the combination therapies comprise the use of L-asparaginase and an inhibitor of both BCL-XL and BCL-2. In further embodiments, the combination shows a synergistic effect against SCLC. In further embodiments, the combination therapy with the synergistic effect against SCLC is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from an SCLC cell line (see for example FIG. 3). In further embodiments, the synergy of the L-asparaginase and an inhibitor of both BCL-XL and BCL-2 is shown in assays involving the SCLC cell line NCI-H146 (see for example FIG. 3) or the SCLC cell line NCI-H69.

In some embodiments, described herein are combination therapies for pancreatic cancer, wherein the combination therapies comprise the use of L-asparaginase and an inhibitor of both BCL-XL and BCL-2. In further embodiments, the combination shows a synergistic effect against pancreatic cancer. In further embodiments, the combination therapy with the synergistic effect against pancreatic cancer is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a pancreatic cell line (see for example FIG. 3). In further embodiments, the synergy of the L-asparaginase and an inhibitor of both BCL-XL and BCL-2 is shown in assays involving the pancreatic cancer cell line MiaPaCa-2 (see for example FIG. 3).

In some embodiments, described herein are combination therapies for breast cancer, wherein the combination therapies comprise the use of L-asparaginase and an inhibitor of both BCL-XL and BCL-2. In further embodiments, the combination shows a synergistic effect against breast cancer. In further embodiments, the combination therapy with the synergistic effect against pancreatic cancer is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a breast cancer cell line (see for example FIG. 3). In further embodiments, the synergy of the L-asparaginase and an inhibitor of both BCL-XL and BCL-2 is shown in assays involving the breast cancer cell lines MDA-MB-231, MDA-MB-453, Hs578T, or MDA-MB-468 (see for example FIG. 3).

In some embodiments, described herein are combination therapies for ovarian cancer, wherein the combination therapies comprise the use of L-asparaginase and an inhibitor of both BCL-XL and BCL-2. In further embodiments, the combination shows a synergistic effect against ovarian cancer. In further embodiments, the combination therapy with the synergistic effect against ovarian cancer is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from an ovarian cell line (see for example FIG. 3). In further embodiments, the synergy of the L-asparaginase and an inhibitor of both BCL-XL and BCL-2 is shown in assays involving the ovarian cancer cell line OVCAR-3 (see for example FIG. 3).

In some embodiments, described herein are combination therapies for sarcoma, wherein the combination therapies comprise the use of L-asparaginase and an inhibitor of both BCL-XL and BCL-2. In further embodiments, the combination shows a synergistic effect against sarcoma. In further embodiments, the combination therapy with the synergistic effect against sarcoma is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a sarcoma cell line (see for example FIG. 3). In further embodiments, the synergy of the L-asparaginase and an inhibitor of both BCL-XL and BCL-2 is shown in assays involving the sarcoma cell line SW982 (see for example FIG. 3).

In some embodiments, described herein are combination therapies for gastric cancer, wherein the combination therapies comprise the use of L-asparaginase and an inhibitor of both BCL-XL and BCL-2. In further embodiments, the combination shows a synergistic effect against gastric cancer. In further embodiments, the combination therapy with the synergistic effect against gastric cancer is a combination comprising L-asparaginase and navitoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a gastric cancer cell line (see for example FIG. 3). In further embodiments, the synergy of the L-asparaginase and an inhibitor of both BCL-XL and BCL-2 is shown in assays involving the gastric cancer cell line KATO III (see for example FIG. 3).

As will be appreciated and as is discussed in further detail herein, any of the above indications treated with any of the L-asparaginase and an inhibitor of both BCL-XL and BCL-2 described herein may be treated by treating with L-asparaginase prior to treating with an inhibitor of both BCL-XL and BCL-2, by treating with L-asparaginase subsequent to treating with an inhibitor of both BCL-XL and BCL-2, or by treating with L-asparaginase simultaneously with an inhibitor of both BCL-XL and BCL-2. Administration may be provided by separate compositions, administration at different times in separate compositions, or administration in a composition in which two or more active pharmaceutical ingredients are present. Simultaneous administration in separate compositions and administration in a composition in which both L-asparaginase and the inhibitor of both BCL-XL and BCL-2 are present is also described herein. Depending on how each one of the active pharmaceutical ingredients is delivered the frequency of dosing may vary. In some embodiments, each or both of the L-asparaginase and the inhibitor of both BCL-XL and BCL-2 can be given every one day, two days, three days, four days, five days, six days, one week, two weeks, three weeks, 1 month, 2 months, or three months. In some embodiments, the inhibitor of both BCL-XL and BCL-2 is administered following administration with L-asparaginase. In some embodiments, the inhibitor of both BCL-XL and BCL-2 is administered before administration with L-asparaginase. In some embodiments, there are at least 3-6 h between the administration of the L-asparaginase and the inhibitor of both BCL-XL and BCL-2. In some embodiments there is one, two, three, four, five, six, seven, eight, nine, ten, eleven, twelve hours between the administration of the L-asparaginase and the inhibitor of both BCL-XL and BCL-2. In some embodiments there are about 24 hours between the administration of the L-asparaginase and the inhibitor of both BCL-XL and BCL-2. In some embodiments there are about 48 hours between the administration of the L-asparaginase and the inhibitor of both BCL-XL and BCL-2. Administration of the L-asparaginase and the inhibitor of both BCL-XL and BCL-2 can be by many different routes, including, but not limited to: intravenous, intraperitoneal, subcutaneous, intramuscular, oral, topical (transdermal), or transmucosal administration. In some embodiments, the route of administration for L-asparaginase and the inhibitor of both BCL-XL and BCL-2 will be the same. In some embodiments, the route of administration for L-asparaginase and the inhibitor of both BCL-XL and BCL-2 will be different. In some embodiments, the route of administration for the inhibitor of both BCL-XL and BCL-2 will be intravenous administration. In some embodiments, the inhibitor of both BCL-XL and BCL-2 will be administered every two weeks. In some embodiments, the inhibitor of both BCL-XL and BCL-2 will be administered every three weeks. In some embodiments, the inhibitor of both BCL-XL and BCL-2 will be administered every four weeks. In some embodiments, the route of administration for the L-asparaginase will be intravenous administration. In some embodiments, the route of administration for the L-asparaginase will be intramuscular administration.

D. L-asparaginase and mTOR Inhibitors

In some embodiments, the uses and methods of treatment of the disclosure comprise administering an L-asparaginase with an inhibitor of mammalian Target of Rapamycin (mTOR). mTOR is a serine/threonine protein kinase which utilizes two protein complexes, mTORC1 and mTORC2 in order to regulate cell growth, proliferation, and metabolism. In some embodiments, inhibiting mTOR inhibits activation of T cells and B cells. In some embodiments, inhibiting mTOR inhibits activation of T cells and B cells by reducing IL-2. In some embodiments, inhibiting mTOR inhibits calcineurin. In some embodiments, mTOR inhibitors stabilize disease. In some embodiments, mTOR inhibitors cause disease regression.

Examples of mTOR inhibitors that can be used in the present disclosure include, but are not limited to: any small molecules that inhibit mTOR, an antibody that inhibits mTOR, an antibody-drug conjugate with a mTOR payload, a dendrimer with a mTOR payload, a prodrug that targets mTOR, a proteolysis targeting chimera (PROTAC) that targets mTOR, or any other modality that targets mTOR. Examples of mTOR inhibitors include but are not limited to: rapamycin (sirolimus), rapalogs (rapamycin derivatives), temsirolimus (CCI-779), everolimus (RAD001), and ridaforolimus (AP-23573).

In some embodiments, combination therapies involving an L-asparaginase and an mTOR inhibitor result in a synergistic effect when administered to treat cancer.

Data showing combination therapies involving an L-asparaginase and an mTOR inhibitor can be found in FIGS. 7, 8, 14, 18, and 19.

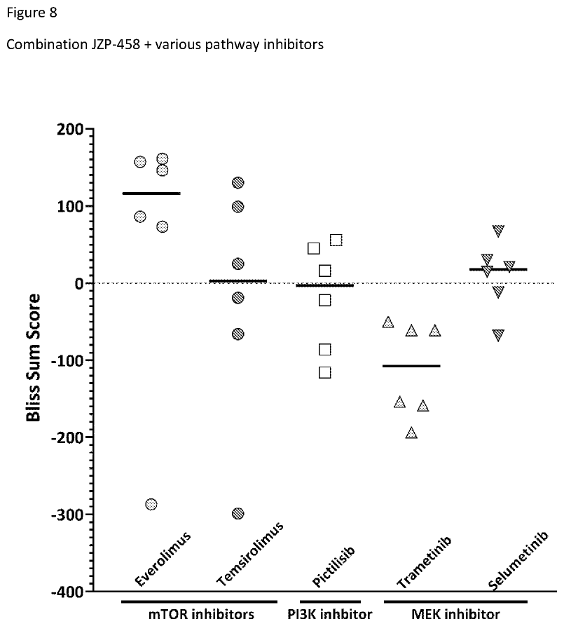

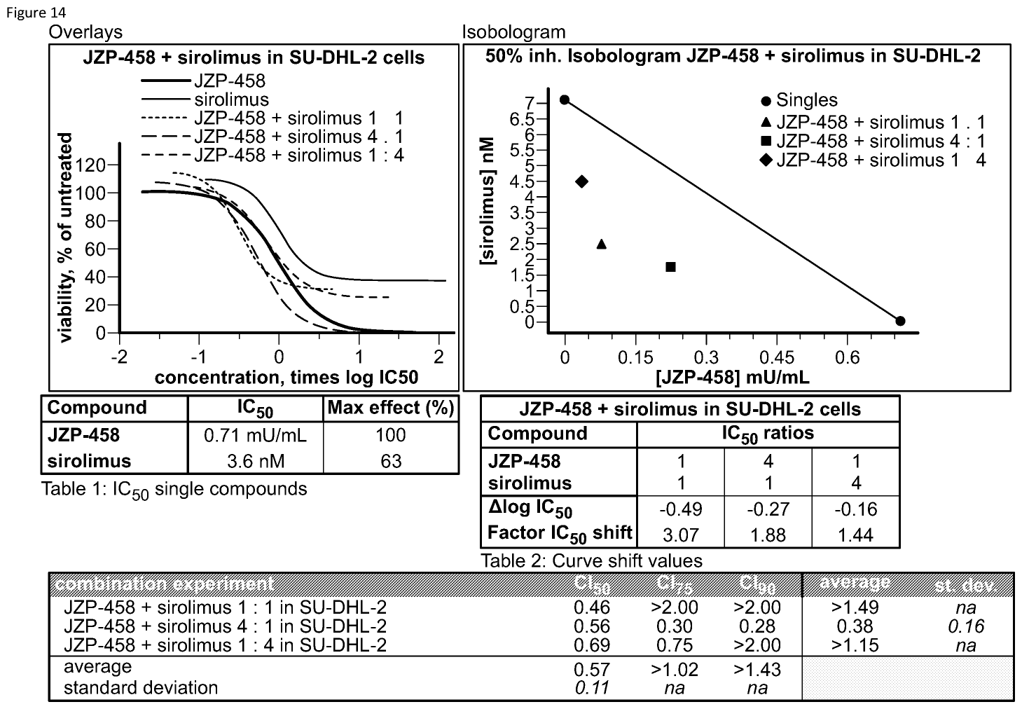

In some embodiments, described herein are combination therapies for diffuse large b cell lymphoma (DLBCL), wherein the combination therapies comprise the use of L-asparaginase and mTOR inhibitors. In further embodiments, the combination shows a synergistic effect against DLBCL. In further embodiments, the combination therapy with the synergistic effect against DLBCL is a combination comprising L-asparaginase and rapamycin (or sirolimus). In other embodiments, the combination therapy with the synergistic effect against DLBCL is a combination comprising L-asparaginase and everolimus. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a DLBCL cell line (see for example FIG. 14). In further embodiments, the synergy of the L-asparaginase and BCL-2 inhibitor is shown in assays involving DLBCL cell lines (see for example FIG. 14). Data in FIG. 8 shows that mTOR inhibitors, specifically everolimus, show an increased Bliss sum score when compared with PI3K inhibitors and MEK inhibitors.

In some embodiments, combination therapies involving an L-asparaginase and everolimus are useful for treatment of patients with unresectably locally advanced or metastatic triple-negative breast cancer (mTNBC). More specifically, combination therapies involving an L-asparaginase and everolimus are useful for treatment of patients with unresectably locally advanced or metastatic triple-negative breast cancer (mTNBC) who have received two or more prior systemic therapies, at least one of them for metastatic disease.

As will be appreciated and as is discussed in further detail herein, any of the above indications treated with any of the L-asparaginase and mTOR inhibitors described herein may be treated by treating with L-asparaginase prior to treating with a mTOR inhibitor, by treating with L-asparaginase subsequent to treating with a mTOR inhibitor, or by treating with L-asparaginase simultaneously with a mTOR inhibitor. Administration may be provided by separate compositions, administration at different times in separate compositions, or administration in a composition in which two or more active pharmaceutical ingredients are present. Simultaneous administration in separate compositions and administration in a composition in which both L-asparaginase and a mTOR inhibitor are present is also described herein. Depending on how each one of the active pharmaceutical ingredients is delivered the frequency of dosing may vary. In some embodiments, each or both of the L-asparaginase and mTOR inhibitor can be given every one day, two days, three days, four days, five days, six days, one week, two weeks, three weeks, 1 month, 2 months, or three months. In some embodiments, the mTOR inhibitor is administered following administration with L-asparaginase. In some embodiments, the mTOR inhibitor is administered before administration with L-asparaginase. In some embodiments, there are at least 3-6 h between the administration of the L-asparaginase and the mTOR inhibitor. In some embodiments there is one, two, three, four, five, six, seven, eight, nine, ten, eleven, twelve hours between the administration of the L-asparaginase and the mTOR inhibitor. In some embodiments there are about 24 hours between the administration of the L-asparaginase and the mTOR inhibitor. In some embodiments there are about 48 hours between the administration of the L-asparaginase and the mTOR inhibitor. Administration of the L-asparaginase and the mTOR inhibitor can be by many different routes, including, but not limited to: intravenous, intraperitoneal, subcutaneous, intramuscular, oral, topical (transdermal), or transmucosal administration. In some embodiments, the route of administration for L-asparaginase and the mTOR inhibitor will be the same. In some embodiments, the route of administration for L-asparaginase and the mTOR inhibitor will be different. In some embodiments, the route of administration for the mTOR inhibitor will be intravenous administration. In some embodiments, the mTOR inhibitor will be administered every two weeks. In some embodiments, the mTOR inhibitor will be administered every three weeks. In some embodiments, the mTOR inhibitor will be administered every four weeks. In some embodiments, the route of administration for the L-asparaginase will be intravenous administration. In some embodiments, the route of administration for the L-asparaginase will be intramuscular administration.

### E. L-asparaginase and CD20 Inhibitors

In some embodiments, the uses and methods of treatment of the disclosure comprise administering an L-asparaginase with a CD20 inhibitor. CD20 is a membrane-embedded surface protein found on lymphocytes, more specifically B cells, which is encoded by the MS4A1 gene. CD20 plays a role promoting the development and differentiation of B cells into plasma cells. CD20 can be overexpressed in certain types of cancer, for example, B-cell lymphomas and leukemias.

Examples of CD20 inhibitors that can be used in the present disclosure include, but are not limited to: any small molecules that inhibit CD20, an antibody that inhibits CD20, an antibody-drug conjugate with a CD20 payload, a dendrimer with a CD20 payload, a prodrug that targets CD20, a proteolysis targeting chimera (PROTAC) that targets CD20, or any other modality that targets CD20. Examples of CD20 inhibitors include but are not limited to: rituximab, ofatumumab, ublituximab, ocrelizumab, obinutuzumab, ocaratuzumab, ibritumomab tiuxetan, tositumomab, TRU-015, and IMMU-106. In some embodiments, the combination therapy involving an L-asparaginase and a CD20 inhibitor includes rituximab, cyclophosphamide, hydroxydaunorubicin hydrochloride (doxorubicin hydrochloride), vincristine (Oncovin) and prednisone (also known as R-CHOP).

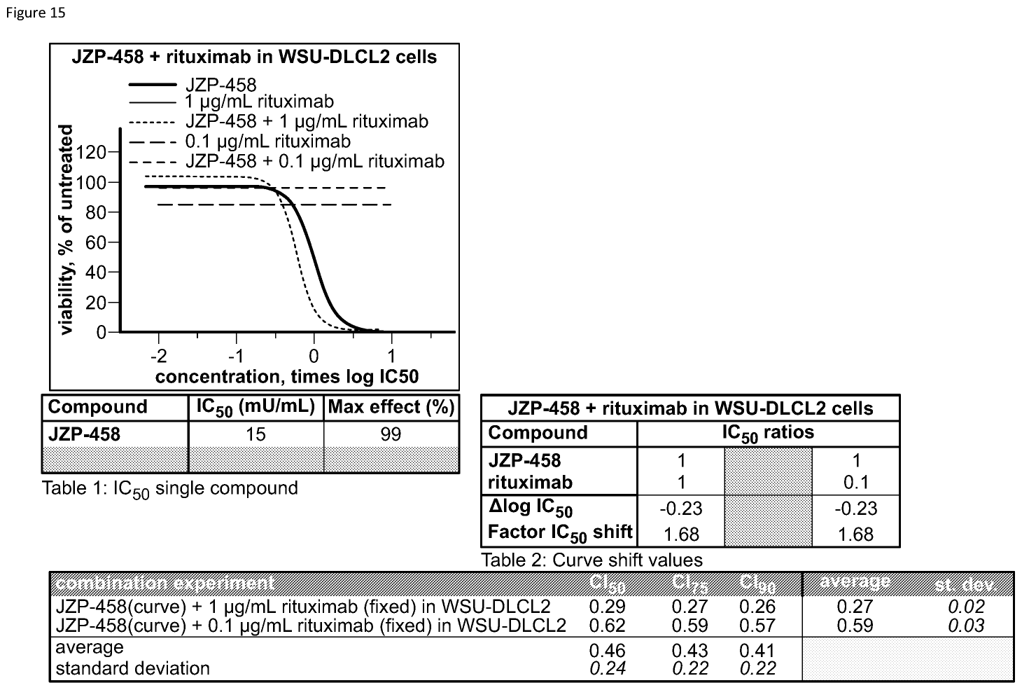

Data showing combination therapies involving an L-asparaginase and an mTOR inhibitor can be found in FIGS. 15, 18, and 19.

In some embodiments, combination therapies involving an L-asparaginase and an CD20 inhibitor result in a synergistic effect when administered to treat cancer.

In some embodiments, described herein are combination therapies for lymphoma, wherein the combination therapies comprise the use of L-asparaginase and CD20 inhibitors. In further embodiments, the combination shows a synergistic effect against lymphoma. In further embodiments, the combination therapy with the synergistic effect against lymphoma is a combination comprising L-asparaginase and rituximab. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a lymphoma cell line (see for example FIG. 15). In further embodiments, the synergy of the L-asparaginase and a CD20 inhibitor is shown in assays involving lymphoma cell lines (see for example FIG. 15).

In some embodiments, described herein are combination therapies for b cell lymphoma, wherein the combination therapies comprise the use of L-asparaginase and CD20 inhibitors. In further embodiments, the combination shows a synergistic effect against b cell lymphoma. In further embodiments, the combination therapy with the synergistic effect against b cell lymphoma is a combination comprising L-asparaginase and rituximab. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a CI50 score from a b cell lymphoma cell line (see for example FIG. 15). In further embodiments, the synergy of the L-asparaginase and a CD20 inhibitor is shown in assays involving b cell lymphoma cell lines WSU-DLCL2 (THL) (see for example FIG. 15).

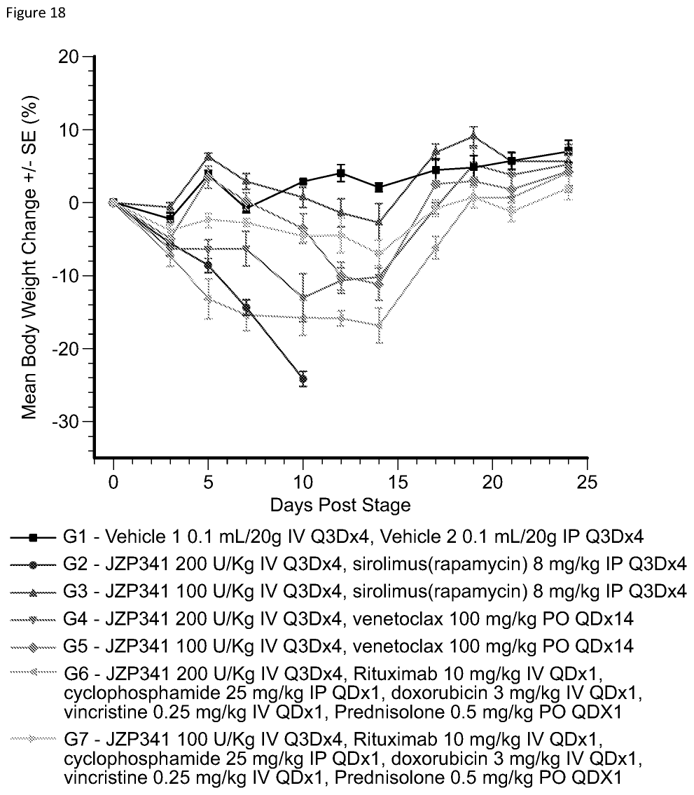

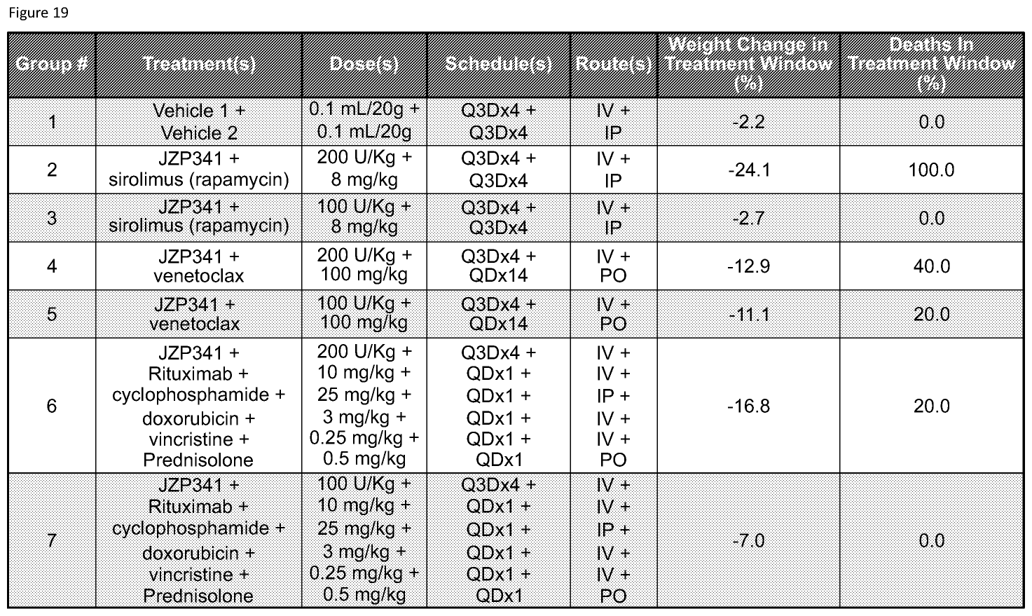

As it was shown herein (e.g., in FIGS. 18 and 19) that coadministration of a CD20 inhibitor increases the efficacy of diffuse large b cell lymphoma (DLBCL) treatment with L-asparaginase, in some embodiments, the present disclosure provides a method of treating DLBCL in a patient including administering to the patient an effective amount of a protein having substantial L-asparagine aminohydrolase activity and a CD20 inhibitor. In some embodiments, the DLBCL is ABC-DLBCL. In some embodiments, the CD20 inhibitor is prednisone or vincristine. In some embodiments, the CD20 inhibitor is prednisone. In some embodiments, the CD20 inhibitor is vincristine. In some embodiments, the L-asparaginase is pasylated. In some embodiments, the L-asparaginase is pegylated. In some embodiments, the L-asparaginase is functionalized with about 5000 Da PEG or mPEG.

In some embodiments, combination therapies involving an L-asparaginase and rituximab can be useful for the treatment of DLBCL. In some embodiments, combination therapies involving an L-asparaginase and rituximab can be useful for the treatment of lymphoma. In some embodiments, combination therapies involving an L-asparaginase and rituximab can be useful for the treatment of B-cell non-Hodgkin lymphoma.

In some embodiments, the combination therapy involving an L-asparaginase and a CD20 inhibitor includes rituximab, cyclophosphamide, hydroxydaunorubicin hydrochloride (doxorubicin hydrochloride), vincristine (Oncovin) and prednisone (also known as R-CHOP). FIGS. 18 and 19 shows data of treatment with JZP341 in combination with R-CHOP, including change in body weight and percent death in treatment window.

As will be appreciated and as is discussed in further detail herein, any of the above indications treated with any of the L-asparaginase and CD20 inhibitors described herein may be treated by treating with L-asparaginase prior to treating with a CD20 inhibitor, by treating with L-asparaginase subsequent to treating with a CD20 inhibitor, or by treating with L-asparaginase simultaneously with a CD20 inhibitor. Administration may be provided by separate compositions, administration at different times in separate compositions, or administration in a composition in which two or more active pharmaceutical ingredients are present. Simultaneous administration in separate compositions and administration in a composition in which both L-asparaginase and a CD20 inhibitor are present is also described herein. Depending on how each one of the active pharmaceutical ingredients is delivered the frequency of dosing may vary. In some embodiments, each or both of the L-asparaginase and CD20 inhibitor can be given every one day, two days, three days, four days, five days, six days, one week, two weeks, three weeks, 1 month, 2 months, or three months. In some embodiments, the CD20 inhibitor is administered following administration with L-asparaginase. In some embodiments, the CD20 inhibitor is administered before administration with L-asparaginase. In some embodiments, there are at least 3-6 h between the administration of the L-asparaginase and the CD20 inhibitor. In some embodiments there is one, two, three, four, five, six, seven, eight, nine, ten, eleven, twelve hours between the administration of the L-asparaginase and the CD20 inhibitor. In some embodiments there are about 24 hours between the administration of the L-asparaginase and the CD20 inhibitor. In some embodiments there are about 48 hours between the administration of the L-asparaginase and the CD20 inhibitor. Administration of the L-asparaginase and the CD20 inhibitor can be by many different routes, including, but not limited to: intravenous, intraperitoneal, subcutaneous, intramuscular, oral, topical (transdermal), or transmucosal administration. In some embodiments, the route of administration for L-asparaginase and the CD20 inhibitor will be the same. In some embodiments, the route of administration for L-asparaginase and the CD20 inhibitor will be different. In some embodiments, the route of administration for the CD20 inhibitor will be intravenous administration. In some embodiments, the CD20 inhibitor will be administered every two weeks. In some embodiments, the CD20 inhibitor will be administered every three weeks. In some embodiments, the CD20 inhibitor will be administered every four weeks. In some embodiments, the route of administration for the L-asparaginase will be intravenous administration. In some embodiments, the route of administration for the L-asparaginase will be intramuscular administration.

F. Other Combinations with L-asparaginase

BTK inhibitors can also be combined with an L-asparaginase for the treatment of cancer. Examples of BTK inhibitors that can be used in the present disclosure include, but are not limited to: any small molecules that inhibit BTK, an antibody that inhibits BTK, an antibody-drug conjugate with a BTK payload, a dendrimer with a BTK payload, a prodrug that targets BTK, a proteolysis targeting chimera (PROTAC) that targets BTK, or any other modality that targets BTK. Examples of BTK inhibitors include but are not limited to: ibrutinib, zanubrutinib, and acalabrutinib.

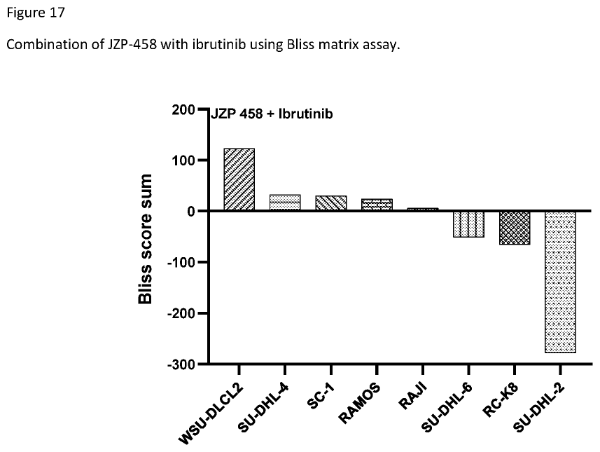

FIG. 17 shows synergy of JZP458 with ibrutinib in lymphoma lines (BLISS matrix). In some embodiments, described herein are combination therapies for lymphoma, wherein the combination therapies comprise the use of L-asparaginase and BTK inhibitors. In further embodiments, the combination shows a synergistic effect against lymphoma. In further embodiments, the combination therapy with the synergistic effect against lymphoma is a combination comprising L-asparaginase and ibrutinib. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a lymphoma cell line (see for example FIG. 17). In further embodiments, the synergy of the L-asparaginase and a BTK inhibitor is shown in assays involving lymphoma cell lines (see for example FIG. 17).

In some embodiments, described herein are combination therapies for b cell lymphoma, wherein the combination therapies comprise the use of L-asparaginase and BTK inhibitors. In further embodiments, the combination shows a synergistic effect against b cell lymphoma. In further embodiments, the combination therapy with the synergistic effect against B cell lymphoma is a combination comprising L-asparaginase and ibrutinib. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a B cell lymphoma cell line (see for example FIG. 17). In further embodiments, the synergy of the L-asparaginase and a BTK inhibitor is shown in assays involving B cell lymphoma cell lines WSU-DLCL2 (THL) (see for example FIG. 17).

As will be appreciated and as is discussed in further detail herein, any of the above indications treated with any of the L-asparaginase and BTK inhibitors described herein may be treated by treating with L-asparaginase prior to treating with a BTK inhibitor, by treating with L-asparaginase subsequent to treating with a BTK inhibitor, or by treating with L-asparaginase simultaneously with a BTK inhibitor. Administration may be provided by separate compositions, administration at different times in separate compositions, or administration in a composition in which two or more active pharmaceutical ingredients are present. Simultaneous administration in separate compositions and administration in a composition in which both L-asparaginase and a BTK inhibitor are present is also described herein. Depending on how each one of the active pharmaceutical ingredients is delivered the frequency of dosing may vary. In some embodiments, each or both of the L-asparaginase and BTK inhibitor can be given every one day, two days, three days, four days, five days, six days, one week, two weeks, three weeks, 1 month, 2 months, or three months. In some embodiments, the BTK inhibitor is administered following administration with L-asparaginase. In some embodiments, the BTK inhibitor is administered before administration with L-asparaginase. In some embodiments, there are at least 3-6 h between the administration of the L-asparaginase and the BTK inhibitor. In some embodiments there is one, two, three, four, five, six, seven, eight, nine, ten, eleven, twelve hours between the administration of the L-asparaginase and the BTK inhibitor. In some embodiments there are about 24 hours between the administration of the L-asparaginase and the BTK inhibitor. In some embodiments there are about 48 hours between the administration of the L-asparaginase and the BTK inhibitor. Administration of the L-asparaginase and the BTK inhibitor can be by many different routes, including, but not limited to: intravenous, intraperitoneal, subcutaneous, intramuscular, oral, topical (transdermal), or transmucosal administration. In some embodiments, the route of administration for L-asparaginase and the BTK inhibitor will be the same. In some embodiments, the route of administration for L-asparaginase and the BTK inhibitor will be different. In some embodiments, the route of administration for the BTK inhibitor will be intravenous administration. In some embodiments, the BTK inhibitor will be administered every two weeks. In some embodiments, the BTK inhibitor will be administered every three weeks. In some embodiments, the BTK inhibitor will be administered every four weeks. In some embodiments, the route of administration for the L-asparaginase will be intravenous administration. In some embodiments, the route of administration for the L-asparaginase will be intramuscular administration.

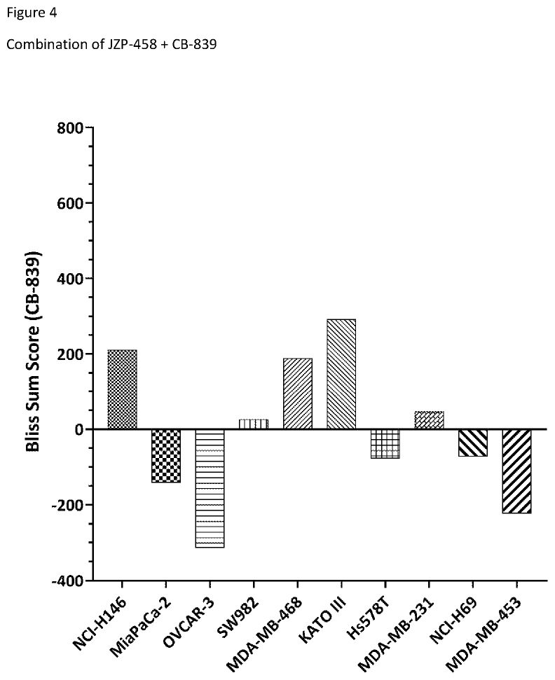

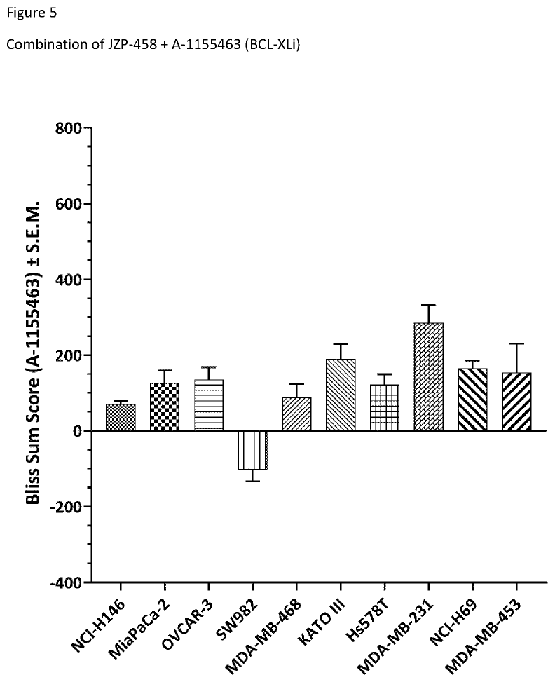

Glutaminase inhibitors can also be combined with an L-asparaginase for the treatment of cancer. An example of a glutaminase inhibitor is CB-839. FIG. 4 shows JZP458 combined with CB-839. Synergy was found for sarcoma cell line SW982 and breast cancer cell line MDA-MB-231. Strong synergy was indicated for lung cancer cell line NCI-146, breast cancer cell line MDA-MB-468, and gastric cancer cell line KATOIII. In some embodiments, the glutaminase inhibitor and L-asparaginase are combined with an inhibitor of BCL-XL.

In some embodiments, described herein are combination therapies for lung cancer, wherein the combination therapies comprise the use of L-asparaginase and glutaminase inhibitors. In further embodiments, the combination shows a synergistic effect against lung cancer. In further embodiments, the combination therapy with the synergistic effect against lung cancer is a combination comprising L-asparaginase and CB-839. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a lung cancer cell line (see for example FIG. 4). In further embodiments, the synergy of the L-asparaginase and a glutaminase inhibitor is shown in assays involving the lung cancer cell line NCI-H146 (see for example FIG. 4).

In some embodiments, described herein are combination therapies for small cell lung cancer (SCLC), wherein the combination therapies comprise the use of L-asparaginase and glutaminase inhibitors. In further embodiments, the combination shows a synergistic effect against SCLC. In further embodiments, the combination therapy with the synergistic effect against SCLC is a combination comprising L-asparaginase and CB-839. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from an SCLC cell line (see for example FIG. 4). In further embodiments, the synergy of the L-asparaginase and a glutaminase inhibitor is shown in assays involving the SCLC cell line NCI-H146 (see for example FIG. 4).

In some embodiments, described herein are combination therapies for breast cancer, wherein the combination therapies comprise the use of L-asparaginase and glutaminase inhibitors. In further embodiments, the combination shows a synergistic effect against breast cancer. In further embodiments, the combination therapy with the synergistic effect against pancreatic cancer is a combination comprising L-asparaginase and CB-839. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a breast cancer cell line (see for example FIG. 4). In further embodiments, the synergy of the L-asparaginase and a glutaminase inhibitor is shown in assays involving the breast cancer cell lines MDA-MB-231 or MDA-MB-468 (see for example FIG. 4).

In some embodiments, described herein are combination therapies for sarcoma, wherein the combination therapies comprise the use of L-asparaginase and glutaminase inhibitors. In further embodiments, the combination shows a synergistic effect against sarcoma. In further embodiments, the combination therapy with the synergistic effect against sarcoma is a combination comprising L-asparaginase and CB-839. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a sarcoma cell line (see for example FIG. 4). In further embodiments, the synergy of the L-asparaginase and a glutaminase inhibitor is shown in assays involving the sarcoma cell line SW982 (see for example FIG. 4).

In some embodiments, described herein are combination therapies for gastric cancer, wherein the combination therapies comprise the use of L-asparaginase and glutaminase inhibitors. In further embodiments, the combination shows a synergistic effect against gastric cancer. In further embodiments, the combination therapy with the synergistic effect against gastric cancer is a combination comprising L-asparaginase and venetoclax. In yet further embodiments, the synergy is seen when the combination therapy includes a recombinant L-asparaginase, including the L-asparaginase referred to as JZP-458, JZP-341, and/or Erwinase. In still further embodiments, the synergistic effect presents as a BLISS score from a gastric cancer cell line (see for example FIG. 4). In further embodiments, the synergy of the L-asparaginase and a glutaminase inhibitor is shown in assays involving the gastric cancer cell line KATO III (see for example FIG. 4).

In some embodiments, the uses and methods of treatment of the disclosure include administering an L-asparaginase with a vascular endothelial growth factor (VEGF) inhibitor. VEGF is a signaling protein that stimulates angiogenesis. Abnormal VEGF function can be a principal inducer of tumor vascularization associated with tumor growth and survival. In the context of L-asparaginase cancer treatment, VEGF inhibition can augment the effects of blood asparagine depletion by further limiting the supply of asparagine to solid tumors. Examples of VEGF inhibitors consistent with the present disclosure include Altiratinib, Anlotinib, Apatinib, Avastin, Axitinib, Bevacizumab, Brivanib, Cabozantinib, Cediranib, Delitinib, Donafenib, Dovitinib, Famitinib, Forctinib, Fruquintinib, Glesatinib, Golvatinib, Ilorasertib, Lenvatinib, Linifanib, Midostaurin, Motesanib, Ningetinib, Nintedanib, Orantinib, Pazopanib, Ponatinib, Ramucirumab, Rebastinib, Regorafenib, Semaxanib, Sorafenib, Sulfatinib, Sunitinib, Tafctinib, Telatinib, Tesevatinib, Tivozanib, Vandetanib, Vatalanib, and Vorolanib. In certain embodiments, the VEGF inhibitor is Avastin or Regorafenib. In particular embodiments, the VEGF inhibitor is a VEGF-A inhibitor.

In some embodiments, the uses and methods of treatment of the disclosure include administering an L-asparaginase with a BRAF inhibitor. BRAF is a protein kinase responsible for transducing a broad set of cellular signals to activate Mitogen-activated protein kinase kinase (MEK) signaling. BRAF constitutive activation and overexpression can induce aberrant cell growth, and can be a primary driver of cancer. BRAF inhibition can augment the effects of L-asparagainse therapy to limit cancer cell growth and survival. Examples of BRAF inhibitors consistent with the present disclosure include Dabrafenib, Encorafenib, Sorafenib, and Vemurafenib. In particular embodiments, the BRAF inhibitor is Encorafenib, or Vemurafenib.

In some embodiments, the uses and methods of treatment of the disclosure include administering an L-asparaginase with a pan-RAF inhibitor. As used herein, the term “pan-RAF inhibitor” can denote species that inhibit two or more of ARAF, BRAF, and CRAF. In some embodiments, a pan-raf inhibitor inhibits all three of ARAF, BRAF, and CRAF. Examples of pan-RAF inhibitors consistent with the present disclosure include Encorafenib, LY-3009120, HM95573 (GDC-5573), LXH-254, MLN2480, BeiGene-283, RXDX-105, BAL3833, Regorafenib, and Sorafenib. In certain embodiments, the pan-raf inhibitor is Encorafenib.

In some embodiments, the uses and methods of treatment of the disclosure include administering an L-asparaginase with a MEK inhibitor. MEK is a central signaling protein in the MAPK signaling pathway that is capable of activating a range of growth and survival responses. Aberrant MEK activity is associated with a large number of cancers, can promote unchecked cancer growth and diminish the efficacy of chemotherapeutic treatment. As disclosed herein, MEK inhibition can synergistically enhance L-asparaginase cancer treatment by attenuating MEK-mediated growth and survival signaling induced by asparagine starvation. Examples of MEK inhibitors consistent with the present disclosure include binimetinib, cobimetinib, selumetinib, and trametinib. In certain embodiments, the MEK inhibitor is binimetinib or cobimetinib.

In some embodiments, the uses and methods of treatment of the disclosure include administering an L-asparaginase with a checkpoint inhibitor. As used herein, the term “checkpoint inhibitor” denotes a species that inhibits or antagonizes an immune checkpoint such as PD-L1 or CTLA-4. As a number of cancers utilize immune checkpoints to generate immunosuppressive environments conducive to growth and metastasis, checkpoint inhibitor therapy can dramatically enhance immune-mediated cancer clearance following L-asparaginase therapy. Examples of checkpoint inhibitors consistent with the present disclosure include and Atezolizumab, avelumab, BMS-936559, Durvalumab, Ipilimumab, Lambrolizumab, MDX1105-01, MEDI4736, MPDL3280A, MSB0010718C, Pembrolizumab, Nivolumab, and Tremelimumab.

As will be appreciated and as is discussed in further detail herein, any of the above indications treated with any of the L-asparaginase and glutaminase inhibitors described herein may be treated by treating with L-asparaginase prior to treating with a glutaminase inhibitor, by treating with L-asparaginase subsequent to treating with a glutaminase inhibitor, or by treating with L-asparaginase simultaneously with a glutaminase inhibitor. Administration may be provided by separate compositions, administration at different times in separate compositions, or administration in a composition in which two or more active pharmaceutical ingredients are present. Simultaneous administration in separate compositions and administration in a composition in which both L-asparaginase and a glutaminase inhibitor are present is also described herein. Depending on how each one of the active pharmaceutical ingredients is delivered the frequency of dosing may vary. In some embodiments, each or both of the L-asparaginase and glutaminase inhibitor can be given every one day, two days, three days, four days, five days, six days, one week, two weeks, three weeks, 1 month, 2 months, or three months. In some embodiments, the glutaminase inhibitor is administered following administration with L-asparaginase. In some embodiments, the glutaminase inhibitor is administered before administration with L-asparaginase. In some embodiments, there are at least 3-6 h between the administration of the L-asparaginase and the glutaminase inhibitor. In some embodiments there is one, two, three, four, five, six, seven, eight, nine, ten, eleven, twelve hours between the administration of the L-asparaginase and the glutaminase inhibitor. In some embodiments there are about 24 hours between the administration of the L-asparaginase and the glutaminase inhibitor. In some embodiments there are about 48 hours between the administration of the L-asparaginase and the glutaminase inhibitor. Administration of the L-asparaginase and the glutaminase inhibitor can be by many different routes, including, but not limited to: intravenous, intraperitoneal, subcutaneous, intramuscular, oral, topical (transdermal), or transmucosal administration. In some embodiments, the route of administration for L-asparaginase and the glutaminase inhibitor will be the same. In some embodiments, the route of administration for L-asparaginase and the glutaminase inhibitor will be different. In some embodiments, the route of administration for the glutaminase inhibitor will be intravenous administration. In some embodiments, the glutaminase inhibitor will be administered every two weeks. In some embodiments, the glutaminase inhibitor will be administered every three weeks. In some embodiments, the glutaminase inhibitor will be administered every four weeks. In some embodiments, the route of administration for the L-asparaginase will be intravenous administration. In some embodiments, the route of administration for the L-asparaginase will be intramuscular administration.

As will be appreciated and as is discussed in further detail herein, any of the above indications treated with any combination of the L-asparaginase, glutaminase inhibitors, and BCL-XL inhibitors described herein may be treated by treating with L-asparaginase prior to treating with a glutaminase inhibitor and/or BCL-XL inhibitors, by treating with L-asparaginase subsequent to treating with a glutaminase inhibitor and/or BCL-XL inhibitors, or by treating with L-asparaginase simultaneously with a glutaminase inhibitor and/or BCL-XL inhibitors. Administration may be provided by separate compositions, administration at different times in separate compositions, or administration in a composition in which two or more active pharmaceutical ingredients are present. Simultaneous administration in separate compositions and administration in a composition in which a L-asparaginase, a glutaminase inhibitor and a BCL-XL inhibitor are present is also described herein. Depending on how each one of the active pharmaceutical ingredients is delivered the frequency of dosing may vary. In some embodiments, each or all of the L-asparaginase, glutaminase inhibitor, and/or BCL-XL inhibitor can be given every one day, two days, three days, four days, five days, six days, one week, two weeks, three weeks, 1 month, 2 months, or three months. In some embodiments, the glutaminase inhibitor is administered following administration with L-asparaginase. In some embodiments, the BCL-XL inhibitor is administered following administration with L-asparaginase. In some embodiments, the glutaminase inhibitor is administered before administration with L-asparaginase. In some embodiments, the BCL-XL inhibitor is administered before administration with L-asparaginase. In some embodiments, there are at least 3-6 h between the administration of the L-asparaginase and the glutaminase inhibitor. In some embodiments, there are at least 3-6 h between the administration of the L-asparaginase and the BCL-XL inhibitor. In some embodiments, there are at least 3-6 h between the administration of the BCL-XL inhibitor and the glutaminase inhibitor. In some embodiments there is one, two, three, four, five, six, seven, eight, nine, ten, eleven, twelve hours between the administration of the L-asparaginase and the glutaminase inhibitor. In some embodiments there is one, two, three, four, five, six, seven, eight, nine, ten, eleven, twelve hours between the administration of the L-asparaginase and the BCL-XL inhibitor. In some embodiments there is one, two, three, four, five, six, seven, eight, nine, ten, eleven, twelve hours between the administration of the BCL-XL and the glutaminase inhibitor. In some embodiments there are about 24 hours between the administration of the L-asparaginase and the glutaminase inhibitor. In some embodiments there are about 24 hours between the administration of the L-asparaginase and the BCL-XL inhibitor. In some embodiments there are about 24 hours between the administration of the BCL-XL inhibitor and the glutaminase inhibitor. In some embodiments there are about 48 hours between the administration of the L-asparaginase and the glutaminase inhibitor. In some embodiments there are about 48 hours between the administration of the L-asparaginase and the BCL-XL inhibitor. In some embodiments there are about 48 hours between the administration of the BCL-XL inhibitor and the glutaminase inhibitor. Administration of the L-asparaginase, the glutaminase inhibitor, and/or the BCL-XL inhibitor can be by many different routes, including, but not limited to: intravenous, intraperitoneal, subcutaneous, intramuscular, oral, topical (transdermal), or transmucosal administration. In some embodiments, the route of administration for L-asparaginase and the glutaminase inhibitor will be the same. In some embodiments, the route of administration for L-asparaginase, the glutaminase inhibitor, and/or the BCL-XL will be different. In some embodiments, the route of administration for the glutaminase inhibitor will be intravenous administration. In some embodiments, the route of administration for the BCL-XL inhibitor will be intravenous administration. In some embodiments, the route of administration for the glutaminase inhibitor will be intramuscular administration. In some embodiments, the route of administration for the BCL-XL inhibitor will be intramuscular administration. In some embodiments, the glutaminase inhibitor will be administered every two weeks. In some embodiments, the glutaminase inhibitor will be administered every three weeks. In some embodiments, the glutaminase inhibitor will be administered every four weeks. In some embodiments, the BCL-XL inhibitor will be administered every two weeks. In some embodiments, the BCL-XL inhibitor will be administered every three weeks. In some embodiments, the BCL-XL inhibitor will be administered every four weeks. In some embodiments, the route of administration for the L-asparaginase will be intravenous administration. In some embodiments, the route of administration for the L-asparaginase will be intramuscular administration.

As will be appreciated and as is discussed in further detail herein, any of the above indications treated with any of the L-asparaginase and an inhibitor described herein can be treated by treating with L-asparaginase prior to treating with an inhibitor, by treating with L-asparaginase subsequent to treating with an inhibitor, or by treating with L-asparaginase simultaneously with an inhibitor.

As will be appreciated and as is discussed herein, any of the above inhibitors can be combined with any of the other inhibitors discussed herein. In some embodiments, more than one inhibitor can be combined with L-asparaginase in order to treat a patient with a disease treatable by asparagine depletion. In some embodiments, more than one inhibitor can be combined with L-asparaginase in order to treat a patient with cancer.

### V. Methods of Treatment and Use of the Combination Therapies Involving a L-asparaginase

### A. Diseases or Disorders

The combination therapies involving L-asparaginase described herein are useful in the treatment of cancer. In some embodiments, the human subject has, prior to administration of a combination therapy involving L-asparaginase, experienced silent inactivation of the E. Coli-derived asparaginase. In some embodiments, the human subject has, prior to administration of a combination therapy involving L-asparaginase experienced an allergic reaction to the E. Coli-derived asparaginase. In some embodiments, the human subject has, prior to administration of a combination therapy involving L-asparaginase, experienced anaphylaxis to the E. Coli-derived asparaginase. Non-limiting examples of objective signs of allergy or hypersensitivity include testing “antibody positive” for an asparaginase enzyme.

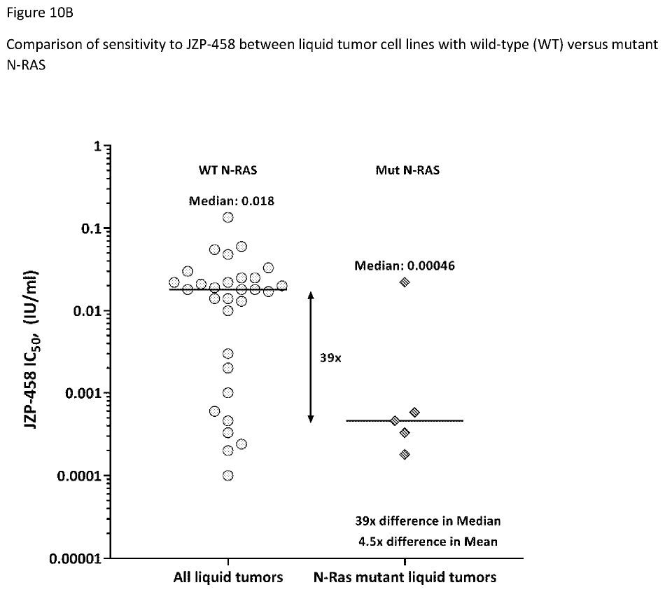

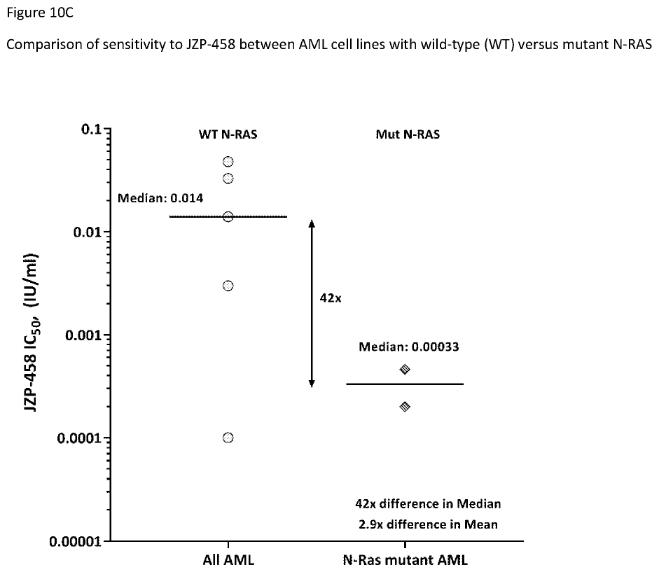

In some embodiments, a combination therapy involving L-asparaginase of the present disclosure can be used in the treatment of cancer. In some embodiments, a combination therapy involving L-asparaginase described herein is useful in the treatment or the manufacture of a medicament for use in the treatment of acute lymphoblastic leukemia (ALL). The incidence of relapse in ALL patients following treatment with L-asparaginase remains high, with approximately 10-25% of pediatric ALL patients having early relapse (e.g., some during maintenance phase at 30-36 months post-induction). If a patient treated with E. coli-derived L-asparaginase has a relapse, subsequent treatment with E. coli preparations could lead to a “vaccination” effect, whereby the E. coli preparation has increased immunogenicity during the subsequent administrations. In one embodiment, a combination therapy involving L-asparaginase is useful in a method of treating patients with relapsed ALL who were previously treated with other asparaginase preparations, in particular those who were previously treated with E. coli-derived asparaginases. In some embodiments, a combination therapy involving L-asparaginase administered to the patient with relapsed ALL includes L-asparaginase conjugated with a PEG moiety. In some embodiments, a combination therapy involving L-asparaginase administered to the patient with relapsed ALL includes an L-asparaginase that is not conjugated with a PEG moiety. In some embodiments, a combination therapy involving L-asparaginase administered to the patient with relapsed ALL includes an L-asparaginase that is conjugated with a proline- or alanine-containing peptide. In some embodiments, a combination therapy involving L-asparaginase administered to the patient with relapsed ALL includes an L-asparaginase that is not conjugated with a proline- or alanine-containing peptide. In some embodiments, the ALL is T-cell ALL. In some embodiments, the ALL is B-cell ALL. In some embodiments, the ALL has a NRAS mutation. As illustrated herein, NRAS mutant AML is surprisingly highly sensitive to unfunctionalized L-asparaginase (e.g., as shown in FIG. 10C), accordingly, in particular embodiments, unfunctionalized L-asparaginase (e.g., L-asparaginase with homology to SEQ ID NO:1) is administered as a monotherapy for treating NRAS mutant AML. Optionally, unfunctionalized L-asparaginase (e.g., L-asparaginase with homology to SEQ ID NO:1) is included in combination therapy. For example, in some embodiments, the L-asparaginase is administered in combination with a pan-RAF inhibitor.

In another aspect, the present disclosure provides a method of treating KRAS mutant acute myeloid leukemia (AML) in a patient including administering to the patient an effective amount of an L-asparaginase. In some embodiments, the L-asparaginase is administered as a monotherapy. In some embodiments, the L-asparaginase is administered as part of a combination therapy. In some embodiments, the L-asparaginase is administered in combination with a pan-RAF inhibitor. In some embodiments, the L-asparaginase is unfunctionalized.

In some embodiments, a combination therapy involving L-asparaginase of the present disclosure is useful in the treatment or the manufacture of a medicament for use in the treatment of lymphoblastic lymphoma (LBL). Similarly to patients with ALL, in some embodiments, a combination therapy involving L-asparaginase administered to the patient with relapsed LBL includes an L-asparaginase that is conjugated with a PEG moiety. In some embodiments, a combination therapy involving L-asparaginase administered to the patient with relapsed LBL includes an L-asparaginase that is not conjugated with a PEG moiety. In some embodiments, a combination therapy involving L-asparaginase administered to the patient with relapsed LBL includes an L-asparaginase that is conjugated with a proline- or alanine-containing peptide. In some embodiments, a combination therapy involving L-asparaginase administered to the patient with relapsed LBL includes an L-asparaginase that is not conjugated with a proline- or alanine-containing peptide.

In some embodiments, a combination therapy involving L-asparaginase described herein is used in the treatment or the manufacture of a medicament for use in the treatment of colorectal cancer (CRC). In some embodiments, a combination therapy involving L-asparaginase administered to the patient with CRC includes an L-asparaginase that is conjugated with a PEG moiety. In some embodiments, a combination therapy involving L-asparaginase administered to the patient with CRC includes an L-asparaginase that is not conjugated with a PEG moiety. In some embodiments, a combination therapy involving L-asparaginase administered to the patient with CRC includes an L-asparaginase that is conjugated with a proline- or alanine-containing peptide. In some embodiments, a combination therapy involving L-asparaginase administered to the patient with CRC includes an L-asparaginase that is not conjugated with a proline- or alanine-containing peptide. In some embodiments, the CRC is Wnt-negative CRC.

In a further embodiment, pasylated L-asparaginase is administered as a monotherapy for treating CRC. In particular embodiments, the CRC is Wnt-negative CRC. As Wnt pathway mutations can activate β-catenin signaling related to asparagine synthetase regulation, L-asparaginase monotherapy was previously viewed as an unlikely treatment for Wnt-negative cancers. However, it was surprisingly determined herein that pasylated L-asparaginase is effective for treating a broad range of colorectal cancers, including Wnt-negative CRC.

In some embodiments, a combination therapy involving L-asparaginase described herein is useful in the treatment or the manufacture of a medicament for use in the treatment of acute myeloid leukemia (AML). In some embodiments, a combination therapy involving L-asparaginase administered to the patient with AML includes an L-asparaginase that is conjugated with a PEG moiety. In some embodiments, a combination therapy involving L-asparaginase administered to the patient with AML includes an L-asparaginase that is not conjugated with a PEG moiety. In some embodiments, a combination therapy involving L-asparaginase administered to the patient with AML includes an L-asparaginase that is conjugated with a proline- or alanine-containing peptide. In some embodiments, a combination therapy involving L-asparaginase administered to the patient with AML includes an L-asparaginase that is not conjugated with a proline- or alanine-containing peptide. In some embodiments, the AML is relapsed/refractory acute myeloid leukemia (R/R AML). In some embodiments, the AML is FLT3 resistant AML. In some embodiments, the AML includes NRAS mutations. NRAS mutations can appear in FLT3 resistant AML. FLT3 treatment emergent RAS/MAPK pathway mutations can appear in 37% of patients. Activating mutations in NRAS can appear in 32% of patients. In some embodiments, the AML includes a KRAS mutation. As disclosed herein, KRAS mutations can appear in 7% of patients.

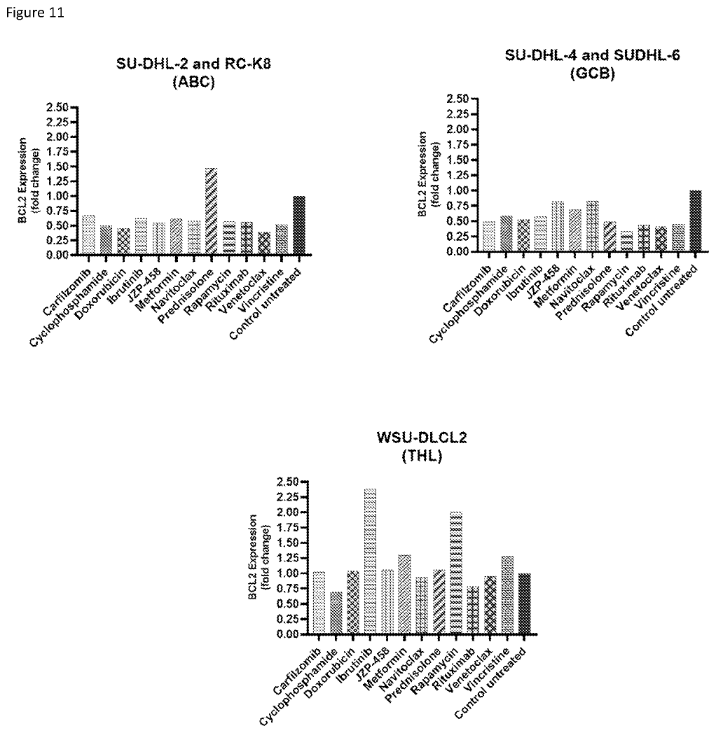

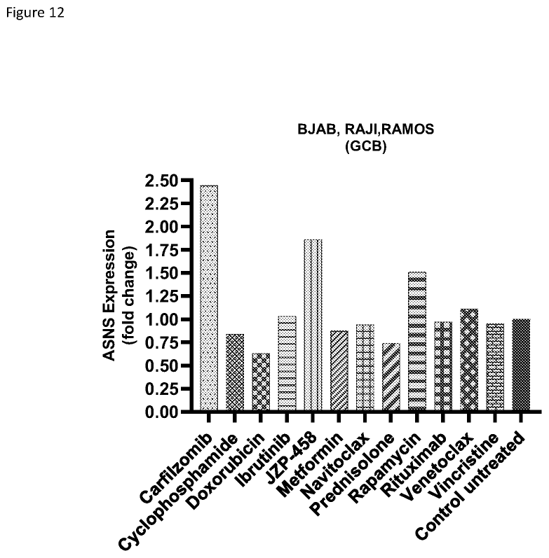

In some embodiments, a combination therapy involving L-asparaginase described herein is useful in the treatment or the manufacture of a medicament for use in the treatment of diffuse large b cell lymphoma (DLBCL). In some embodiments, a combination therapy involving L-asparaginase administered to the patient with DLBCL includes an L-asparaginase that is conjugated with a PEG moiety. In some embodiments, a combination therapy involving L-asparaginase administered to the patient with DLBCL includes an L-asparaginase that is not conjugated with a PEG moiety. In some embodiments, a combination therapy involving L-asparaginase administered to the patient with DLBCL includes an L-asparaginase that is conjugated with a proline- or alanine-containing peptide. In some embodiments, a combination therapy involving L-asparaginase administered to the patient with DLBCL includes an L-asparaginase that is not conjugated with a proline- or alanine-containing peptide. FIG. 19 shows strong sensitivity of DLBCL lines SU-DHL-2, RC-K8, and SU-DHL-6 to L-asparaginase (JZP458). ABC-DLBCL lines and one of GCB-DLBCL line are very sensitive to JZP458. FIG. 11 shows decreased BCL2 expression as prognostic marker for DLBCL lines (ABC and GCB type). JZP458 and most chemotherapeutic agents decrease BCL2 expression. FIG. 12 shows increased BCL2 expression and increased ASNS expression as resistance marker for DLBCL lines (triple hit lymphoma) and Burkitt lymphoma. Ibrutinib and rapamycin significantly increase BCL2 expression in triple hit lymphoma line WSU-DLCL2 and carfilzomib and JZP458 ASNS in Burkitt lymphoma lines. FIG. 19 shows strong sensitivity of DLBCL lines to JZP458. ABC-DLBCL lines and one of GCB-DLBCL lines are very sensitive to JZP458 in the range of 0.011-7.3 IU/mL. Dose-response curve overlays-72 hrs incubation.

In some embodiments, a combination therapy involving L-asparaginase of the present disclosure is useful in the treatment or the manufacture of a medicament for use in the treatment of pancreatic cancer. In some embodiments, a combination therapy involving L-asparaginase administered to the patient with pancreatic cancer includes an L-asparaginase that is conjugated with a PEG moiety. In some embodiments, a combination therapy involving L-asparaginase administered to the patient with pancreatic cancer includes an L-asparaginase that is not conjugated with a PEG moiety. In some embodiments, a combination therapy involving L-asparaginase administered to the patient with pancreatic cancer includes an L-asparaginase that is conjugated with a proline- or alanine-containing peptide. In some embodiments, a combination therapy involving L-asparaginase administered to the patient with pancreatic cancer includes an L-asparaginase that is not conjugated with a proline- or alanine-containing peptide.

In some embodiments, a combination therapy involving L-asparaginase of the present disclosure is useful in the treatment or the manufacture of a medicament for use in the treatment of sarcoma. In some embodiments, the sarcoma is synovial sarcoma. In some embodiments, the sarcoma is osteosarcoma. In some embodiments, the sarcoma is fibrosarcoma. In some embodiments, a combination therapy involving L-asparaginase administered to the patient with pancreatic cancer includes an L-asparaginase that is conjugated with a PEG moiety. In some embodiments, a combination therapy involving L-asparaginase administered to the patient with pancreatic cancer includes an L-asparaginase that is not conjugated with a PEG moiety. In some embodiments, a combination therapy involving L-asparaginase administered to the patient with pancreatic cancer includes an L-asparaginase that is conjugated with a proline- or alanine-containing peptide. In some embodiments, a combination therapy involving L-asparaginase administered to the patient with pancreatic cancer includes an L-asparaginase that is not conjugated with a proline- or alanine-containing peptide.

In some embodiments, a combination therapy involving L-asparaginase of the present disclosure is useful in the treatment or the manufacture of a medicament for use in the treatment of ovarian cancer. In some embodiments, the sarcoma is fibrosarcoma. In some embodiments, a combination therapy involving L-asparaginase administered to the patient with pancreatic cancer includes an L-asparaginase that is conjugated with a PEG moiety. In some embodiments, a combination therapy involving L-asparaginase administered to the patient with pancreatic cancer includes an L-asparaginase that is not conjugated with a PEG moiety. In some embodiments, a combination therapy involving L-asparaginase administered to the patient with pancreatic cancer includes an L-asparaginase that is conjugated with a proline- or alanine-containing peptide. In some embodiments, a combination therapy involving L-asparaginase administered to the patient with pancreatic cancer includes an L-asparaginase that is not conjugated with a proline- or alanine-containing peptide.

In some embodiments, a combination therapy involving L-asparaginase of the present disclosure is useful in the treatment or the manufacture of a medicament for use in the treatment of breast cancer. In some embodiments, the breast cancer is triple negative breast cancer (TNBC). In some embodiments, a combination therapy involving L-asparaginase administered to the patient with pancreatic cancer includes an L-asparaginase that is conjugated with a PEG moiety. In some embodiments, a combination therapy involving L-asparaginase administered to the patient with pancreatic cancer includes an L-asparaginase that is not conjugated with a PEG moiety. In some embodiments, a combination therapy involving L-asparaginase administered to the patient with pancreatic cancer includes an L-asparaginase that is conjugated with a proline- or alanine-containing peptide. In some embodiments, a combination therapy involving L-asparaginase administered to the patient with pancreatic cancer includes an L-asparaginase that is not conjugated with a proline- or alanine-containing peptide.

In some embodiments, a combination therapy involving L-asparaginase of the present disclosure is useful in the treatment or the manufacture of a medicament for use in the treatment of gastric cancer. In some embodiments, a combination therapy involving L-asparaginase administered to the patient with pancreatic cancer includes an L-asparaginase that is conjugated with a PEG moiety. In some embodiments, a combination therapy involving L-asparaginase administered to the patient with pancreatic cancer includes an L-asparaginase that is not conjugated with a PEG moiety. In some embodiments, a combination therapy involving L-asparaginase administered to the patient with pancreatic cancer includes an L-asparaginase that is conjugated with a proline- or alanine-containing peptide. In some embodiments, a combination therapy involving L-asparaginase administered to the patient with pancreatic cancer includes an L-asparaginase that is not conjugated with a proline- or alanine-containing peptide.

In some embodiments, a combination or monotherapy involving L-asparaginase described herein is useful in the treatment or manufacture of a medicament for use in the treatment of brain cancer. In some embodiments, a combination or monotherapy involving L-asparaginase described herein is useful in the treatment or the manufacture of a medicament for use in the treatment of glioblastoma or astrocytoma. In some embodiments, a combination or monotherapy involving L-asparaginase described herein is useful in the treatment or the manufacture of a medicament for use in the treatment of glioblastoma. In some embodiments, a combination or monotherapy involving L-asparaginase described herein is useful in the treatment or the manufacture of a medicament for use in the treatment of astrocytoma. In some embodiments, a combination or monotherapy involving L-asparaginase administered to the patient with glioblastoma or astrocytoma includes an L-asparaginase that is conjugated with a PEG moiety. In some embodiments, a combination or monotherapy involving L-asparaginase administered to the patient with glioblastoma or astrocytoma includes an L-asparaginase that is not conjugated with a PEG moiety. In some embodiments, a combination or monotherapy involving L-asparaginase administered to the patient with glioblastoma or astrocytoma includes an L-asparaginase that is conjugated with a proline- or alanine-containing peptide. In some embodiments, a combination or monotherapy involving L-asparaginase administered to the patient with glioblastoma or astrocytoma includes an L-asparaginase that is not conjugated with a proline- or alanine-containing peptide.

In some embodiments, a combination therapy involving L-asparaginase described herein is useful in the treatment or the manufacture of a medicament for use in the treatment of Burkitt lymphoma. In some embodiments, a combination therapy involving L-asparaginase administered to the patient with Burkitt lymphoma includes an L-asparaginase that is conjugated with a PEG moiety. In some embodiments, a combination therapy involving L-asparaginase administered to the patient with Burkitt lymphoma includes an L-asparaginase that is not conjugated with a PEG moiety. In some embodiments, a combination therapy involving L-asparaginase administered to the patient with Burkitt lymphoma includes an L-asparaginase that is conjugated with a proline- or alanine-containing peptide. In some embodiments, a combination therapy involving L-asparaginase administered to the patient with Burkitt lymphoma includes an L-asparaginase that is not conjugated with a proline- or alanine-containing peptide. FIG. 15 shows increased BCL2 expression and increased ASNS expression as resistance marker for Burkitt lymphoma and carfilzomib and JZP458 increase the ASNS expression in Burkitt lymphoma lines. In some embodiments, a combination therapy involving L-asparaginase described herein is useful in the treatment or the manufacture of a medicament for use in the treatment of EBV-related Burkitt lymphoma.

In some embodiments, a combination or monotherapy involving L-asparaginase described herein is useful in the treatment or the manufacture of a medicament for use in the treatment of liquid tumors comprising an NRAS mutation. In some embodiments, a combination or monotherapy involving L-asparaginase is administered to the patient with a liquid tumor comprising an NRAS mutation includes an L-asparaginase that is conjugated with a PEG moiety. In some embodiments, a combination or monotherapy involving L-asparaginase administered to the patient with a liquid tumor comprising an NRAS mutation includes an L-asparaginase that is not conjugated with a PEG moiety. In some embodiments, a combination or monotherapy involving L-asparaginase administered to the patient with a liquid tumor comprising an NRAS mutation includes an L-asparaginase that is conjugated with a proline- or alanine-containing peptide. In some embodiments, a combination or monotherapy involving L-asparaginase administered to the patient with a liquid tumor comprising an NRAS mutation includes an L-asparaginase that is not conjugated with a proline- or alanine-containing peptide. In some embodiments, a combination or monotherapy involving L-asparaginase administered to the patient with a liquid tumor comprising an NRAS mutation includes an unfunctionalized L-asparaginase. FIGS. 11A, 11B, and 11C show liquid tumors with NRAS mutations appear to be more sensitive to asparaginase (JZP-458). Across the panel sensitivity difference due to NRAS mutations is driven by liquid tumors. AML: 9/34 (26%); T-cell ALL: 6/15 (40%); B-cell ALL: 1/12 (8.3%). FIG. 12 shows NRAS mutations appear in FLT3 resistant AML. FLT3 treatment emergent RAS/MAPK pathway mutations in 37% of patients. Activating mutations in NRAS in 32% of patients. KRAS mutations in 7% of patients. No patients at baseline had detectable NRAS/KRAS mutations.

In some embodiments, a combination or monotherapy involving L-asparaginase described herein is useful in the treatment or the manufacture of a medicament for use in the treatment of liquid tumors comprising a KRAS mutation. In some embodiments, a combination or monotherapy involving L-asparaginase is administered to the patient with a liquid tumor comprising a KRAS mutation includes an L-asparaginase that is conjugated with a PEG moiety. In some embodiments, a combination or monotherapy involving L-asparaginase administered to the patient with a liquid tumor comprising a KRAS mutation includes an L-asparaginase that is not conjugated with a PEG moiety. In some embodiments, a combination or monotherapy involving L-asparaginase administered to the patient with a liquid tumor comprising a KRAS mutation includes an L-asparaginase that is conjugated with a proline- or alanine-containing peptide. In some embodiments, a combination or monotherapy involving L-asparaginase administered to the patient with a liquid tumor comprising a KRAS mutation includes an L-asparaginase that is not conjugated with a proline- or alanine-containing peptide.

In some embodiments, a combination or monotherapy involving L-asparaginase described herein is useful in the treatment or the manufacture of a medicament for use in the treatment of liquid tumors comprising a MAPK pathway mutation. Non-limiting examples of MAPK pathway mutations include NRAS mutation, HRAS mutations, KRAS mutations, ARAF mutations, BRAF mutations, CRAF mutations, MEK mutations, ERK1/2 mutations, and CREB mutations. In some embodiments, a combination or monotherapy involving L-asparaginase is administered to the patient with a liquid tumor comprising a MAPK pathway mutation includes an L-asparaginase that is conjugated with a PEG moiety. In some embodiments, a combination or monotherapy involving L-asparaginase administered to the patient with a liquid tumor comprising a MAPK pathway mutation includes an L-asparaginase that is not conjugated with a PEG moiety. In some embodiments, a combination or monotherapy involving L-asparaginase administered to the patient with a liquid tumor comprising a MAPK pathway mutation includes an L-asparaginase that is conjugated with a proline- or alanine-containing peptide. In some embodiments, a combination or monotherapy involving L-asparaginase administered to the patient with a liquid tumor comprising a MAPK pathway mutation includes an L-asparaginase that is not conjugated with a proline- or alanine-containing peptide.

Diseases or disorders that the L-asparaginase of the present disclosure is also useful in treating include but are not limited to the following: malignancies, or cancers, including but not limited to hematalogic malignancies, lymphoma, non-Hodgkin's lymphoma, B-cell non-Hodgkin lymphoma, double hit lymphoma, NK lymphoma, pancreatic cancer, Hodgkin's disease, large cell immunoblastic lymphoma, acute promyelocytic leukemia, acute myelocytic leukemia, acute myelomonocytic leukemia, acute monocytic leukemia, acute T-cell leukemia, biphenotypic B-cell myelomonocytic Leukemia, chronic lymphocytic leukemia, lymphosarcoma, reticulosarcoma, and melanosarcoma.

Other diseases or disorders that the L-asparaginase is useful in treating are cancers including, but not limited to, carcinoma, renal cell carcinoma, renal cell adenocarcinoma, brain cancer, glioblastoma including glioblastoma multiforma and glioblastoma astrocytoma, medulloblastoma, rhabdomyosarcoma, malignant melanoma, epidermoid carcinoma, squamous cell carcinoma, lung cancer, lung carcinoma including large cell lung carcinoma and small cell lung carcinoma, endometrial carcinoma, ovarian adenocarcinoma, ovarian tetratocarcinoma, cervical adenocarcinoma, breast carcinoma, breast adenocarcinoma, breast ductal carcinoma, pancreatic adenocarcinoma, pancreatic ductal carcinoma, colon carcinoma, colon adenocarcinoma, colorectal adenocarcinoma, bladder transitional cell carcinoma, bladder papilloma, prostate carcinoma, osteosarcoma, epitheloid carcinoma of the bone, prostate carcinoma, and thyroid cancer. In some embodiments, the cancer to be treated is gastric cancer, sarcoma, synovial sarcoma, fibrosarcoma, osteoscarcoma, ovarian cancer, renal cancer, endometrial cancer, sarcomas, bladder cancer, prostate cancer, lymphoma, pancreatic cancer, brain cancer, lung cancer, breast cancer, and colorectal cancer. In some embodiments, the cancer to be treated is lymphoma. In some embodiments, the cancer to be treated is b cell lymphoma. In some embodiments, the cancer to be treated is large cell lymphoma. The cancer may be a solid cancer, for example lung cancer or breast cancer. Cells suspected of causing disease can be tested for asparagine dependence in any suitable in vitro or in vivo assay, e.g., an in vitro assay wherein the growth medium lacks asparagine.

In some embodiments, the cancer is a brain cancer or a cancer of the central nervous system. As used herein, the term “cancer of the central nervous system” (or “CNS cancer”) denotes a cancer of the brain or spinal cord. Examples of CNS cancers include glioblastoma, glioblastoma multiforme, astrocytoma, medulloblastoma, neuroepithelial cancer, oligodendroglioma, ependymoma, oligoastrocytomas, neuroblastoma, gangliogliomas, and gangliocytoma. In particular embodiments, unfunctionalized L-asparaginase is administered for treating a CNS cancer. In particular embodiments, the L-asparaginase is administered as a monotherapy. In some embodiments, the CNS cancer is a brain cancer. In some embodiments, the CNS cancer is a medulloblastoma or glioblastoma. In some embodiments, the CNS cancer is an astrocytoma.

In some embodiments, the cancer is a solid cancer. It was surprisingly shown herein that unfunctionalized L-asparaginase is effective for treating numerous solid cancers (e.g., as disclosed in FIG. 10A). Following from this observation, in some embodiments, unfunctionalized L-asparaginase is administered as a monotherapy for treating a solid cancer.

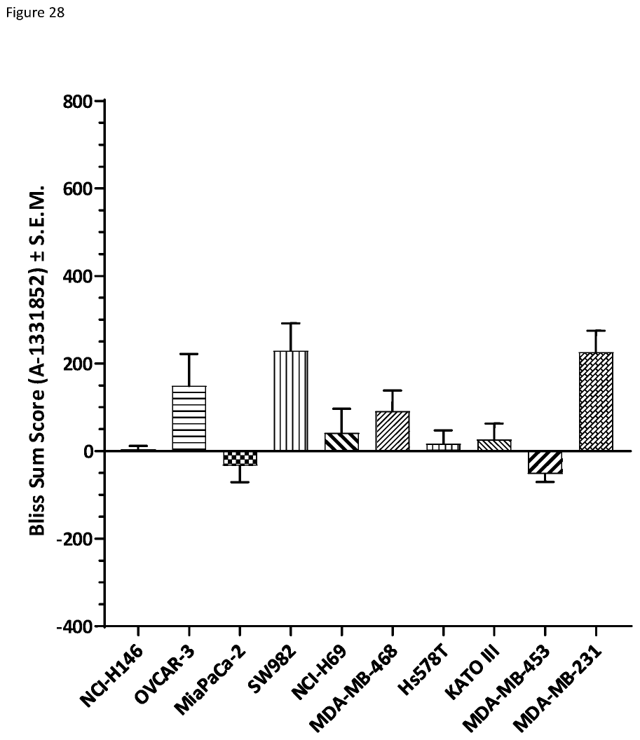

It was further shown herein that the combination of L-asparaginase with a BCL-XL inhibitor is effective for treating solid tumors. As shown in FIGS. 1 and 28, pasylated and unfunctionalized L-asparaginase exhibit synergism with the BCL-XL inhibitor A-1331852 against multiple solid tumor cancers, including a number of lung and breast cancer cells lines. Accordingly, in some embodiments, L-asparaginase is administered with a BCL-XL inhibitor to treat a solid cancer. In some embodiments, the L-asparaginase is pasylated. In some embodiments, the L-asparaginase is unfunctionalized.

Diseases or disorders that the L-asparaginase of the present disclosure is useful in treating include sarcoma, breast cancer, metastatic breast cancer, liver cancer, stomach cancer, colorectal cancer, and head and neck cancer.

### B. Methods for Testing for Asparagine Dependence

Cells suspected of causing disease can be tested for asparagine dependence in any suitable in vitro or in vivo assay, e.g., an in vitro assay wherein the growth medium lacks asparagine. Thus, in some embodiments, the present disclosure is directed to a method of treating a disease treatable in a patient, the method comprising administering to the patient an effective amount of a L-asparaginase described herein. In some embodiments, the L-asparaginase is a recombinant L-asparaginase. In a specific embodiment, the disease is ALL. In a specific embodiment, the disease is LBL. In a particular embodiment, the L-asparaginase used in the treatment of a disease treatable by asparagine depletion comprises the sequence of SEQ ID NO:1. In a further embodiment, the L-asparaginase is not conjugated to a polymer such as PEG.

### C. Methods for Assessing Nadir Serum Asparaginase Activity (NSAA)

Assays for measuring nadir serum asparaginase activity (NSAA) in human subjects can be conducted for evaluation of the human subject. In some embodiments, a serum sample is taken from the human subject to assess NSAA. In some embodiments, a whole blood sample is taken from the subject in order to assess NSAA. In some embodiments, assessing NSAA occurs before the patient is given L-asparaginase. In some embodiments, assessing NSAA occurs after the patient is given L-asparaginase.

### D. Line of Therapy for Combination Therapy Including an L-asparaginase

A first line therapy is the first treatment given for a disease. A first line therapy can be a monotherapy or a standard set of treatments.

A second line therapy can be a monotherapy or a standard set of treatments. A second line therapy is a treatment given after a first treatment fails, loses its effect (either partially or totally), has side effects that are not tolerated, the patient elects to withdraw from the first treatment for any reason, or a new treatment becomes available that can have a better outcome than the present treatment. In some embodiments, the second line therapy can be given to the human subject in addition to the first line therapy for beneficial additive or synergistic results.

Additional lines of therapy including third, fourth, fifth, sixth, and any further lines of therapies are defined similarly to second line therapies but in this case both the first and the second line therapies either fail, lose their effect (either partially or totally), have side effects that are not tolerated, the patient elects to withdraw from the first and/or second lines of therapy for any reason, a new treatment becomes available that can have a better outcome than the first and second line treatment, or any combination of these reasons. Additional lines of therapy can be a monotherapy or a standard set of treatments. In some embodiments, the additional lines of therapy can be given to the human subject in addition to the first line and/or second line of therapy for beneficial additive or synergistic results.

In some embodiments, treatment with a combination therapy comprising an L-asparaginase of the present disclosure will be administered as a first line therapy. In other embodiments, treatment with a combination therapy including an L-asparaginase of the present disclosure will be administered as a second line therapy in patients, particularly patients with ALL and LBL, where objective signs of allergy or hypersensitivity, including “silent inactivation,” have developed to other asparaginase preparations, in particular, the native Escherichia-coli-derived L-asparaginase or its PEGylated variant (pegaspargase). Non-limiting examples of objective signs of allergy or hypersensitivity include testing “antibody positive” for an asparaginase enzyme. The patient can have a previous hypersensitivity to at least one L-asparaginase from E. coli, and/or can have a previous hypersensitivity to at least one L-asparaginase from Erwinia chrysanthemi. The hypersensitivity can be selected from the group consisting of allergic reaction, anaphylactic shock, and silent inactivation. In a specific embodiment, the combination therapy including an L-asparaginase of the present disclosure is used in second line therapy after treatment with pegaspargase. In a more specific embodiment, the combination therapy including an L-asparaginase of the present disclosure used in second line therapy comprises an L-asparaginase produced in a Pseudomonadales flourescens cell, more specifically, comprising a tetramer, wherein each monomer or subunit comprises the sequence of SEQ ID NO: 1. In some embodiments, combination therapy including an L-asparaginase of the present disclosure is used in second line therapy with patients who are hypersensitive to an E. coli-derived L-asparaginase, and/or can have a previous hypersensitivity to an Erwinia chrysanthemi-derived L-asparaginase. An E. coli-derived L-asparaginase can be in the native form or a long-acting form of an E. coli-derived L-asparaginase. The native E. coli-derived L-asparaginase can be identified by SEQ ID NO: 3 or any of the following examples including, but not limited to, Crastinin (Bayer), Elspar (Merck), Kidrolase (Rhone-Poulenc) and Leunase (Kyowa) (Also see ASPG2_ECOLI, Uniprot accession number P00805, https://www.uniprot.org/uniprot/P00805).

SEQ ID NO: 3 is as follows:

MEFFKKTALAALVMGFSGAALALPNITILATGGTIAGGGDSATKS
NYTVGKVGVENLVNAVPQLKDIANVKGEQVVNIGSQDMNDNVWLT
LAKKINTDCDKTDGFVITHGTDTMEETAYFLDLTVKCDKPVVMVG
AMRPSTSMSADGPFNLYNAVVTAADKASANRGVLVVMNDTVLDGR
DVTKTNTTDVATFKSVNYGPLGYIHNGKIDYQRTPARKHTSDTPF
DVSKLNELPKVGIVYNYANASDLPAKALVDAGYDGIVSAGVGNGN
LYKSVEDTLATAAKTGTAVVRSSRVPTGATTQDAEVDDAKYGFVA
SGTLNPQKARVLLQLALTQTKDPQQIQQIFNQY

In some embodiments, the combination therapy comprising an L-asparaginase can be used as a second line therapy with patients receiving a native E. coli-derived asparaginase. In some embodiments, one dose of the L-asparaginase in the combination therapy comprising an L-asparaginase is administered to the patient as a substitute for one dose of the native E. coli-derived asparaginase. In some embodiments, combination therapy comprising an L-asparaginase may be used as a second line therapy with patients receiving a long-acting E. coli-derived asparaginase. In some embodiments, six doses of the L-asparaginase in the combination therapy comprising an L-asparaginase are administered to the patient as a substitute for one dose of the long-acting E. coli-derived asparaginase. In some embodiments, seven doses of the L-asparaginase combination therapy comprising an L-asparaginase are administered to the patient as a substitute for one dose of the long-acting E. coli-derived asparaginase. In some embodiments, the long-acting E. coli-derived asparaginase is pegaspargase. In some embodiments, the long-acting E. coli-derived asparaginase is calaspargase (See Liu, G., Space for Calaspargase? A New Asparaginase for Acute Lymphoblastic Leukemia Clin Cancer Res 2020; 26:325-7). In some embodiments, treatment with a combination therapy comprising an L-asparaginase of the present disclosure is administered as a third line therapy. In some embodiments, treatment with a combination therapy comprising an L-asparaginase of the present disclosure is administered as a fourth line therapy. In some embodiments, treatment with a combination therapy comprising an L-asparaginase of the present disclosure is administered as a fifth line therapy. In some embodiments, treatment with a combination therapy comprising an L-asparaginase of the present disclosure is administered as a sixth line therapy. In some embodiments, treatment with a combination therapy comprising an L-asparaginase of the present disclosure is administered as a maintenance therapy. In some embodiments, administering the combination therapy comprising an L-asparaginase to a patient as a substitute for native or long-acting E. coli-derived asparaginase can occur anytime within the treatment cycle of the native or long-acting E. coli-derived asparaginase. In some embodiments, the treatment cycle for the native or long-acting E. coli-derived asparaginase is anywhere from 1 to 14 doses. In some embodiments, the treatment cycle for the native or long-acting E. coli-derived asparaginase is 1, 2, 3, 4, 5, 6, 7, 8, 9, 10, 11, 12, 13, or 14 doses. In some embodiments, the treatment cycle for the native or long-acting E. coli-derived asparaginase is between 2 and 12 doses. In some embodiments, the treatment cycle for the native or long-acting E. coli-derived asparaginase is between 4 and 10 doses. In some embodiments, the treatment cycle for the native or long-acting E. coli-derived asparaginase is between 4 and 10 doses. In some embodiments, the treatment cycle for the native or long-acting E. coli-derived asparaginase is between 4 and 8 doses. In some embodiments, the treatment cycle for the native or long-acting E. coli-derived asparaginase is between 4 and 6 doses. In some embodiments, administering the combination therapy including an L-asparaginase to a patient as a substitute for native or long-acting E. coli-derived asparaginase can occur anytime within the treatment cycle of the native or long-acting E. coli-derived asparaginase depending on when the patient experiences hypersensitivity.

### VI. Preferred Combinations Including L-asparaginase

In some embodiments, the present disclosure provides for a method of treating cancer in a human subject. In some embodiments, the present disclosure provides a method of treating cancer in a patient comprising administering to the patient an effective amount of an L-asparaginase and an inhibitor of BCL-XL. In some embodiments, the BCL-XL inhibitor is selected from the group consisting of: a small molecule that inhibit BCL-XL, an antibody that inhibits BCL-XL, an antibody-drug conjugate with a BCL-XL payload, a dendrimer with a BCL-XL payload, a prodrug that targets BCL-XL, and a proteolysis targeting chimera (PROTAC) that targets BCL-XL. In some embodiments, BCL-XL inhibitor is selected from the group consisting of: BCL-XL inhibitor is selected from the group consisting of: A-1155463, A-1331852, WEHI-539, WEHI-539 HCl, BH3I-1, A-1293102, DT2216, XZ424, XZ739, PZ15227, PROTAC 1, and ABBV-155. In some embodiments, the BCL-XL inhibitor is A-1331852. In some embodiments, the BCL-XL inhibitor is A-1155463. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is newly diagnosed AML. In some embodiments, the cancer is lymphoma. In some embodiments, the lymphoma is b cell lymphoma. In some embodiments, the lymphoma is DLBCL. In some embodiments, the lymphoma is Burkitt lymphoma. In some embodiments, the lymphoma is EBV-related Burkitt lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method of treatment comprises using ASNS or BCL2 as a prognostic marker to determine treatment. In some embodiments, the method comprises using a Wnt mutation as a prognostic marker to determine treatment.

In some embodiments, the present disclosure provides a method of treating cancer in a patient comprising administering to the patient an effective amount of an L-asparaginase and an inhibitor of BCL-2. In some embodiments, the BCL-2 inhibitor is selected from the group consisting of: a small molecule that inhibits BCL-2, an antibody that inhibits BCL-2, an antibody-drug conjugate with a BCL-2 payload, a dendrimer with a BCL-2 payload, a prodrug that targets BCL-2, and a proteolysis targeting chimera (PROTAC) that targets BCL-2. In some embodiments, the BCL-2 inhibitor is selected from the group consisting of: venetoclax (ABT-199), S55746, BDA-366, oblimersen (G3139), obatoclax, obatoclax mesylate (GX15-070), HA14-1, mifepristone (RU486), TCPOBOP, cinobufagin, nodakenetin (NANI), and motixafortide (BL-8040). In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is b cell lymphoma. In some embodiments, the lymphoma is DLBCL. In some embodiments, the lymphoma is Burkitt lymphoma. In some embodiments, the lymphoma is EBV-related Burkitt lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method of treatment comprises using ASNS or BCL2 as a prognostic marker to determine treatment. In some embodiments, the method comprises using a Wnt mutation as a prognostic marker to determine treatment.

In some embodiments, the present disclosure provides a method of treating cancer in a patient comprising administering to the patient an effective amount of an L-asparaginase and an inhibitor of both BCL-XL and BCL-2. In some embodiments, the both BCL-XL and BCL-2 inhibitor is selected from the group consisting of: a small molecule that inhibits both BCL-XL and BCL-2, an antibody that inhibits both BCL-XL and BCL-2, an antibody-drug conjugate with both a BCL-XL and BCL-2 payload, a dendrimer with both a BCL-XL and BCL-2 payload, a prodrug that targets both BCL-XL and BCL-2, and a proteolysis targeting chimera (PROTAC) that targets both BCL-XL and BCL-2. In some embodiments the inhibitor of both BCL-XL and BCL-2 is selected from the group consisting of: navitoclax (ABT-263), ABT-737, sabutoclax, gossypol, (R)-(−)-gossypol acetic acid, TW-37, gambogic acid, 2-Methoxy-antimycin A, berberine chloride (NSC 646666), berberine chloride hydrate, APG-1252, AZD-0466, BM-1197, AZD4320, and pelcitoclax (APG-1252). In some embodiments, the both BCL-XL and BCL-2 inhibitor is navitoclax. In some embodiments, the both BCL-XL and BCL-2 inhibitor is ABT-737. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is b cell lymphoma. In some embodiments, the lymphoma is DLBCL. In some embodiments, the lymphoma is Burkitt lymphoma. In some embodiments, the lymphoma is EBV-related Burkitt lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method of treatment comprises using ASNS or BCL2 as a prognostic marker to determine treatment. In some embodiments, the method comprises using a Wnt mutation as a prognostic marker to determine treatment.

In some embodiments, the present disclosure provides a method of treating cancer in a patient comprising administering to the patient an effective amount of an L-asparaginase and an mTOR inhibitor. In some embodiments, the mTOR inhibitor is selected from the group consisting of: a small molecule that inhibits mTOR, an antibody that inhibits mTOR, an antibody-drug conjugate with a mTOR payload, a dendrimer with a mTOR payload, a prodrug that targets mTOR, and a proteolysis targeting chimera (PROTAC) that targets mTOR. In some embodiments, the mTOR inhibitor is selected from the group consisting of: rapamycin (sirolimus), rapalogs (rapamycin derivatives), temsirolimus (CCI-779), everolimus (RAD001), and ridaforolimus (AP-23573). In some embodiments, the mTOR inhibitor is rapamycin. In some embodiments, the mTOR inhibitor is rapamycin (sirolimus). In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is b cell lymphoma. In some embodiments, the lymphoma is DLBCL. In some embodiments, the lymphoma is Burkitt lymphoma. In some embodiments, the lymphoma is EBV-related Burkitt lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method of treatment comprises using ASNS or BCL2 as a prognostic marker to determine treatment. In some embodiments, the method comprises using a Wnt mutation as a prognostic marker to determine treatment. In some embodiments, the present disclosure provides a method of treating cancer in a patient comprising administering to the patient an effective amount of an L-asparaginase and an anti-CD20 inhibitor. In some embodiments, the CD20 inhibitor is selected from the group consisting of: a small molecule that inhibits CD20, an antibody that inhibits CD20, an antibody-drug conjugate with a CD20 payload, a dendrimer with a CD20 payload, a prodrug that targets CD20, and a proteolysis targeting chimera (PROTAC) that targets CD20. In some embodiments, the CD20 inhibitor is selected from the group consisting of: rituximab, ofatumumab, ublituximab, ocrelizumab, obinutuzumab, ocaratuzumab, ibritumomab tiuxetan, tositumomab, TRU-015, IMMU-106, and R-CHOP. In some embodiments, the CD20 inhibitor is rituximab. In certain embodiments, the CD20 inhibitor comprises R-CHOP. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is b cell lymphoma. In some embodiments, the lymphoma is DLBCL. In some embodiments, the lymphoma is Burkitt lymphoma. In some embodiments, the lymphoma is EBV-related Burkitt lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method of treatment comprises using ASNS or BCL2 as a prognostic marker to determine treatment. In some embodiments, the method comprises using a Wnt mutation as a prognostic marker to determine treatment.

In some embodiments, the present disclosure provides a method of treating cancer in a patient comprising administering to the patient an effective amount of an L-asparaginase and a BTK inhibitor. In some embodiments, the BTK inhibitor is selected from the group consisting of: a small molecule that inhibits BTK, an antibody that inhibits BTK, an antibody-drug conjugate with a BTK payload, a dendrimer with a BTK payload, a prodrug that targets BTK, and a proteolysis targeting chimera (PROTAC) that targets BTK. In some embodiments, the BTK inhibitor is selected from the group consisting of: ibrutinib, zanubrutinib, and acalabrutinib. In some embodiments, the L-asparaginase is a recombinant L-asparaginase. In some embodiments, the L-asparaginase has at least about 80%, 85%, 90%, 91%, 92%, 93%, 94%, 95%, 96%, 97%, 98%, 99%, or 100% sequence identity to the amino acid of SEQ ID NO: 1. In some embodiments, the L-asparaginase is conjugated with a PEG moiety. In some embodiments, the L-asparaginase is conjugated with a proline- or alanine-containing peptide. In some embodiments, the cancer is acute lymphoblastic leukemia (ALL) or lymphoblastic lymphoma (LBL). In some embodiments, the cancer is colorectal cancer (CRC). In some embodiments, the CRC is Wnt-negative CRC. In some embodiments, the cancer is acute myeloid leukemia (AML). In some embodiments, the AML is R/R AML. In some embodiments, the AML is FLT3 resistant AML. In some embodiments, the cancer is lymphoma. In some embodiments, the lymphoma is b cell lymphoma. In some embodiments, the lymphoma is DLBCL. In some embodiments, the lymphoma is Burkitt lymphoma. In some embodiments, the lymphoma is EBV-related Burkitt lymphoma. In some embodiments, the cancer is breast cancer. In some embodiments, the breast cancer is TNBC. In some embodiments, the cancer is pancreatic cancer. In some embodiments, the cancer is brain cancer. In some embodiments, the cancer is glioblastoma. In some embodiments, the cancer is astrocytoma. In some embodiments, the cancer is lung cancer. In some embodiments, the cancer is small cell lung cancer (SCLC). In some embodiments, the cancer is ovarian cancer. In some embodiments, the cancer is sarcoma. In some embodiments, the cancer is gastric cancer. In some embodiments, the method of treatment comprises using ASNS or BCL2 as a prognostic marker to determine treatment. In some embodiments, the method comprises using a Wnt mutation as a prognostic marker to determine treatment.

### EXAMPLES

### Example 1: Combination Treatment with L-asparaginase

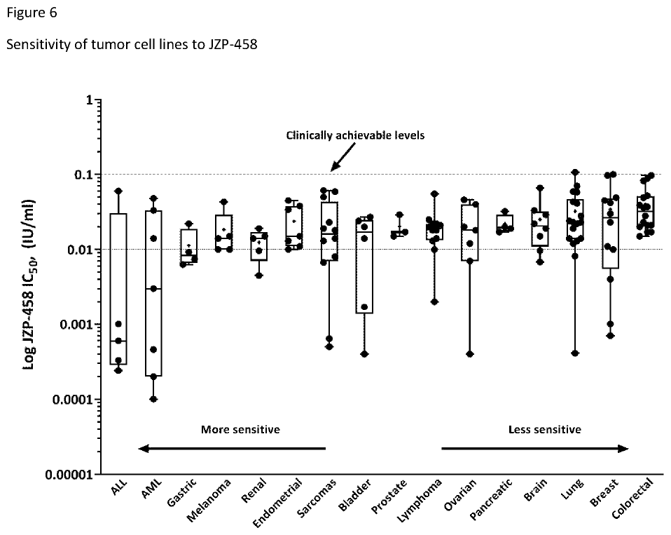

Sensitivity to L-asparaginase was determined in 140 lines across multiple indications. In silico tandem perturbation effects of Asparagine synthetase and signaling components analyzed (≈700 lines). A subset of lines are ASNS dependent for survival (see McDonald, E. Robert et al. (2017). Project DRIVE: A Compendium of Cancer Dependencies and Synthetic Lethal Relationships Uncovered by Large-Scale, Deep RNAi Screening. Cell 170, 577-592 and https://oncologynibr.shinyapps.io/drive/which is herein incorporated by reference to show expression of ASNS and sensitivity to asparagine depletion). In vitro, most lines are sensitive to asparagine depletion within a clinically achievable range (see FIG. 6).

### Procedure:

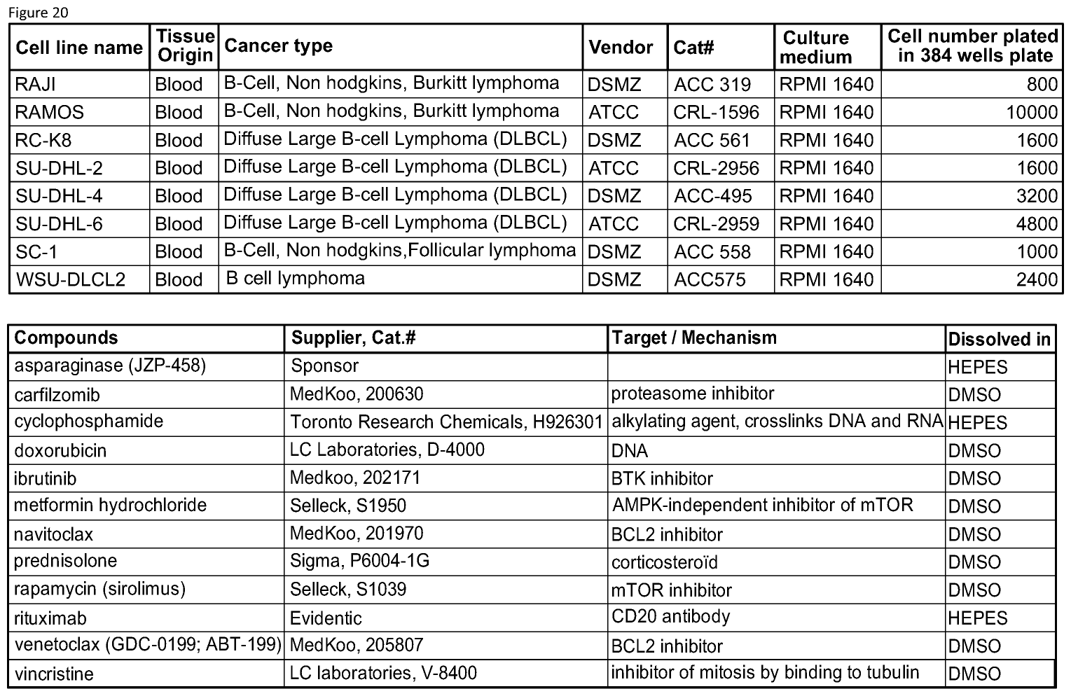

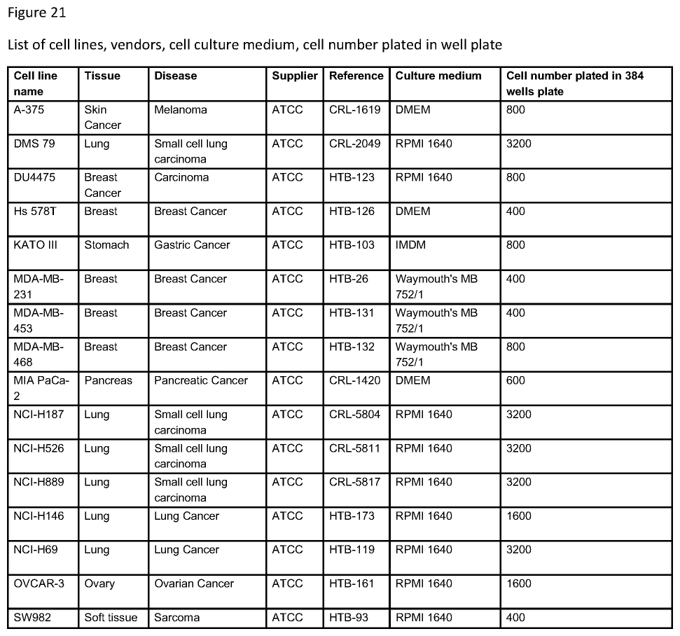

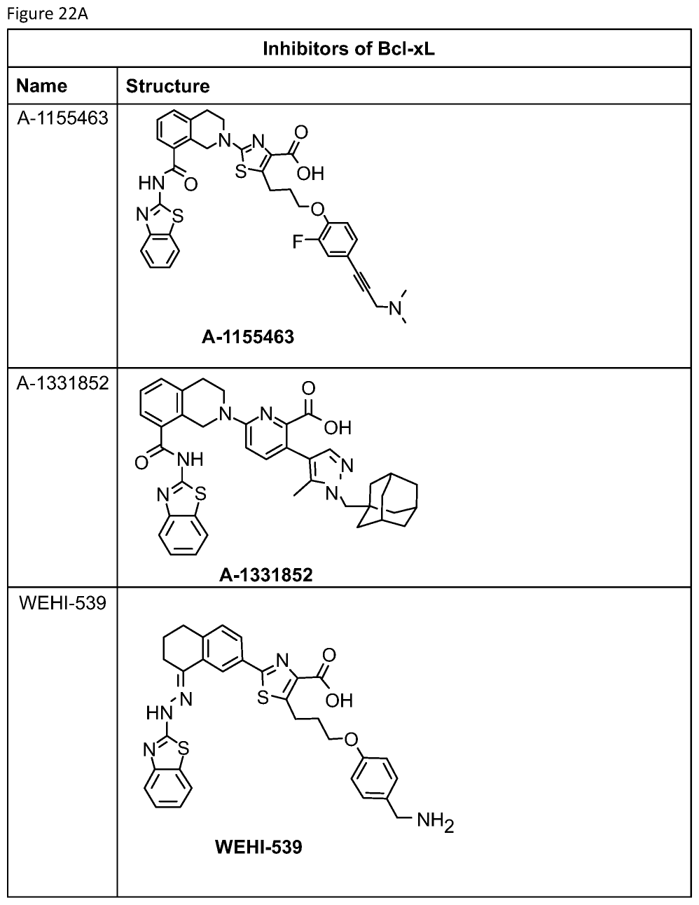

Reagents and Cell lines: See FIG. 20 and FIG. 21

Combination study under fixed ratio (SynergyFinder): SynergyFinder Protocol. Cells were plated in 384-well plates in 45 μl at a density of 2400/well in 45 μl cell culture medium. For each cell line, the optimal cell density was determined a priori to obtain an optimal assay window. Plated cells were incubated in a humidified incubator with 5% CO2 at 37° C. After 24 hours, 5 μl of the appropriate compound combination or single agent was added and plates were further incubated. After 72 hours, plates were cooled in 30 minutes to room temperature and 24 μl of ATPlite 1 Step™ (PerkinElmer) solution was added to each well, and subsequently shaken for 2 minutes. After 5 minutes of incubation in the dark at room temperature, the luminescence was recorded on an Envision multimode reader (PerkinElmer). Synergy was determined by the Combination Index (CI) (Zhao, L., et al., Clin. Cancer Res. 10:7994; 2004) and curve-shift analysis (Chou, T. C., Cancer Res. 70:440; 2010), which also provides a visual representation of synergy. The CI evaluates the concentrations needed to achieve a fixed-effect. A CI of below 1 indicates synergy. A CI of less than 0.3 indicates strong synergy (Haagensen, E. J., et al., Br. J. Cancer 106:1386; 2012). For example, a CI of 0.1 indicates that the combination needs a ten-fold lower concentration than expected from the single agent data, to achieve the same effect level.

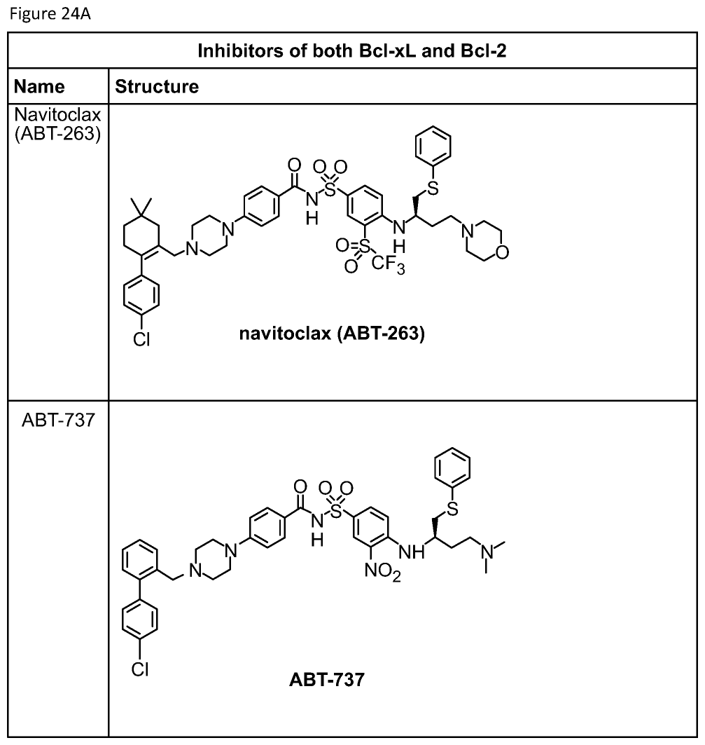

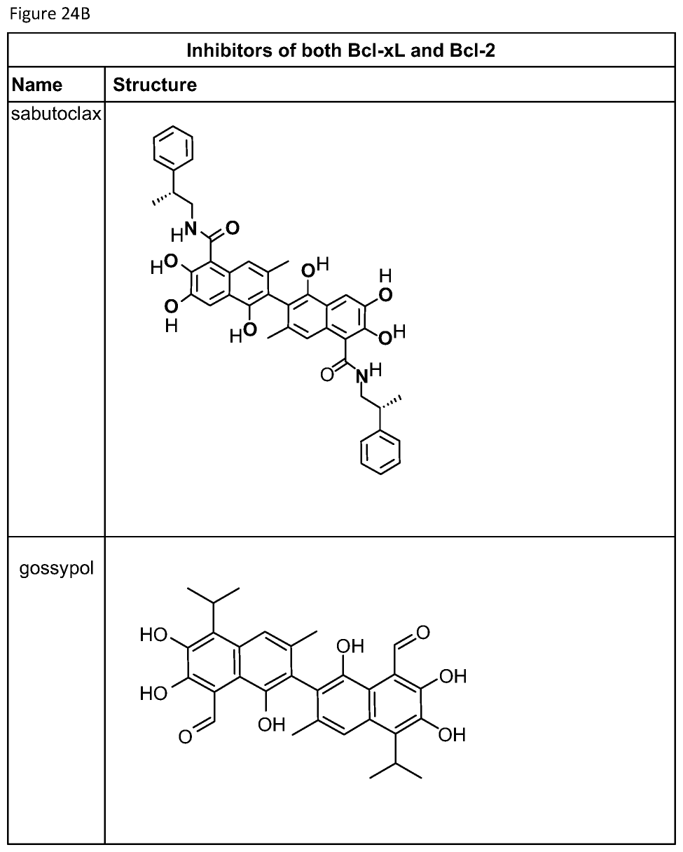

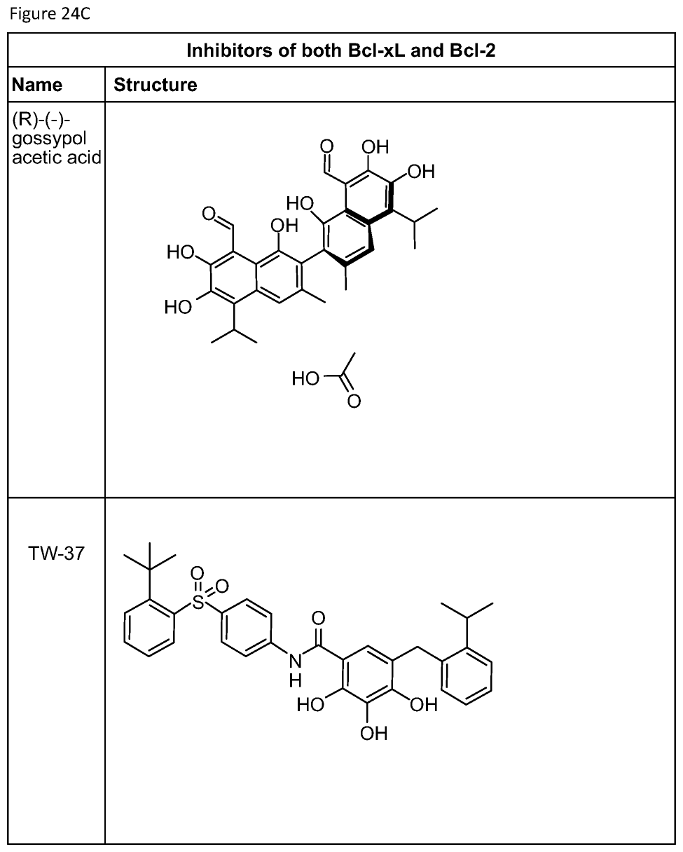

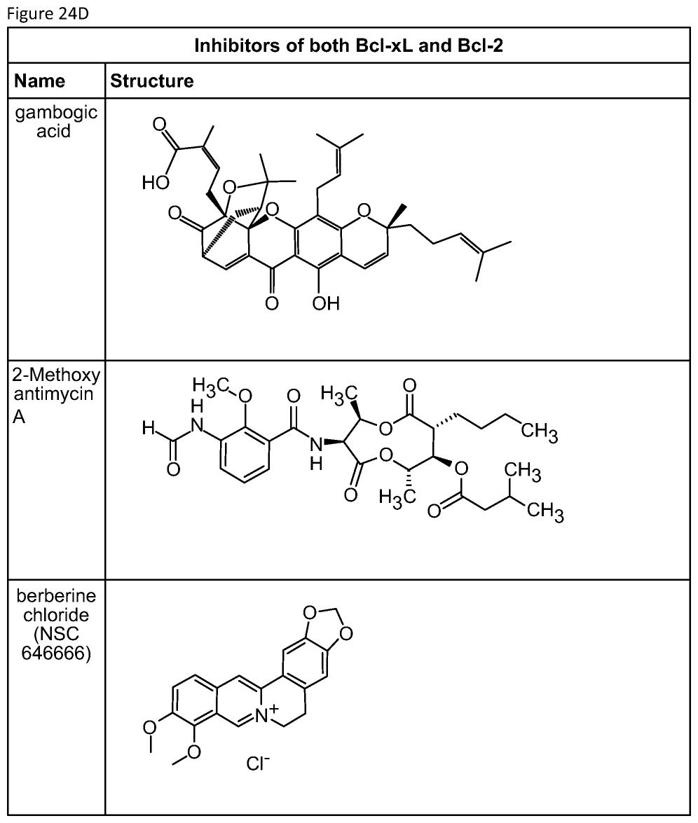

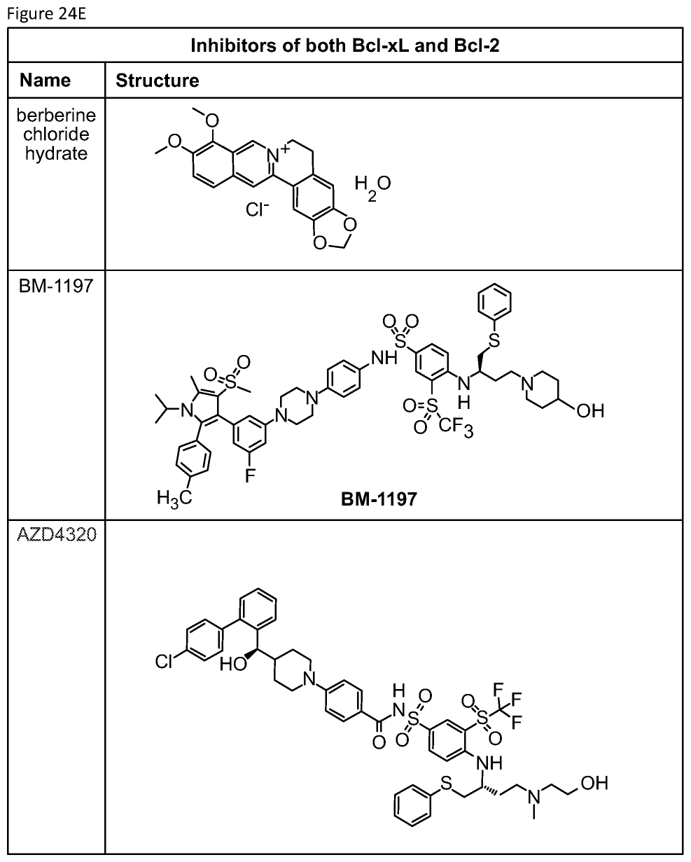

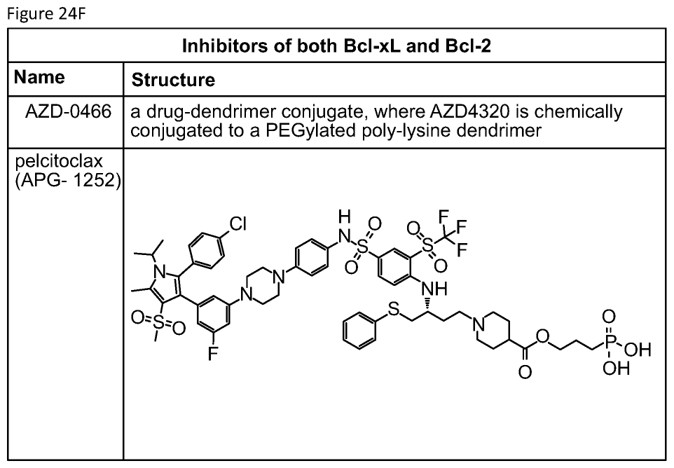

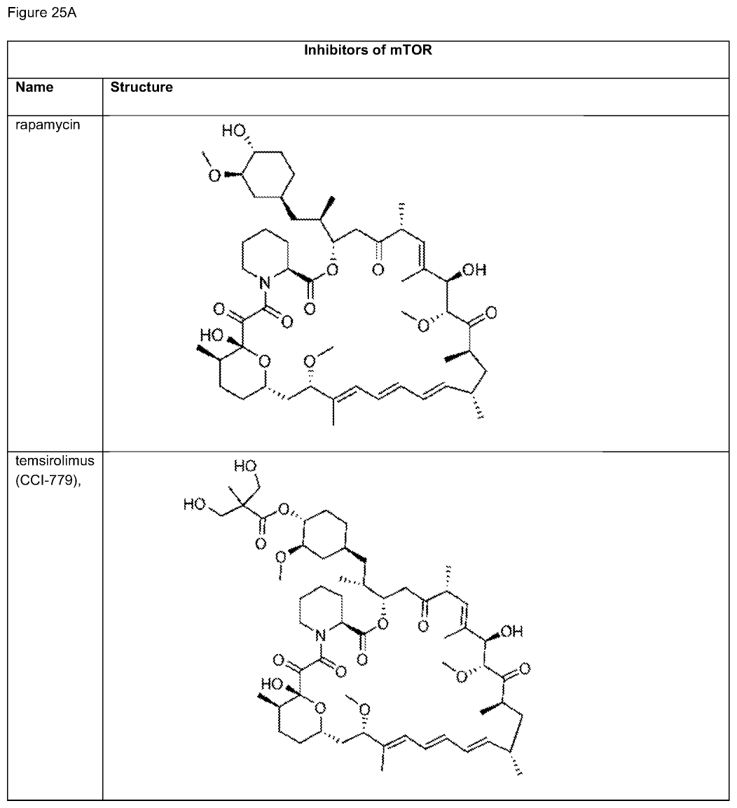

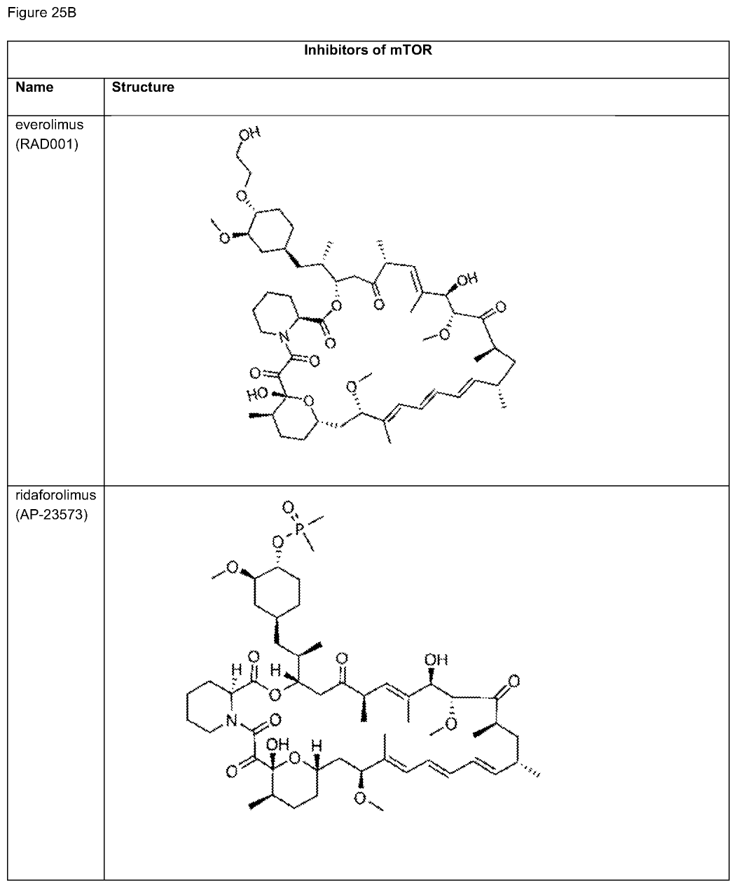

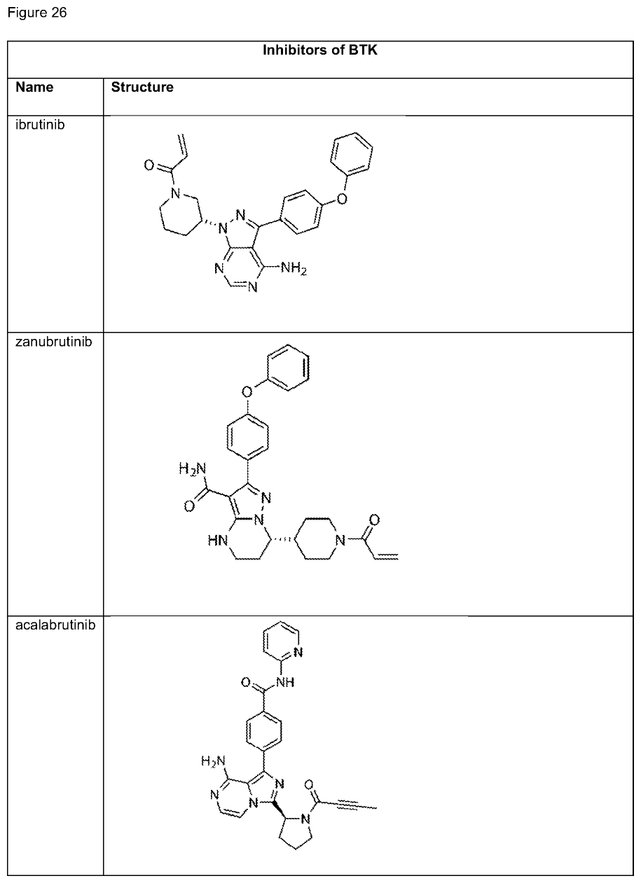

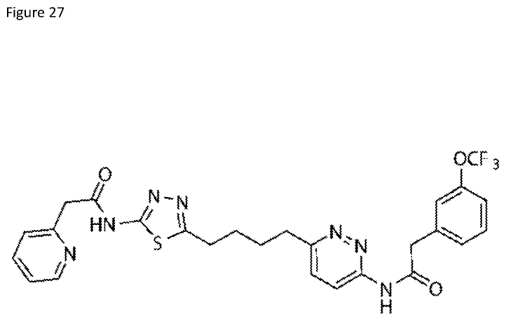

Combination study BLISS matrix-Bliss Assay Protocol: Cells were plated in 384-well plates in 45 μl cell culture medium at a density of, that is, at numbers of cells per well as indicated in FIG. 24. For each cell line, the optimal cell density was determined a priori to obtain an optimal assay window. Plated cells were incubated in a humidified incubator with 5% CO2 at 37° C. 24 hours post-plating, 5 μl of compound dilution was added to each well and plates were further incubated for another 72 hours after which 25 μl of ATPlite 1 Step (PerkinElmer, Groningen, The Netherlands) solution was added to each well. Luminescence was recorded on an Envision multimode reader (PerkinElmer, Waltham, MA, U.S.A.). For determination of Bliss independence scores, two drugs were added in increasing concentrations in a 6×6 matrix. The top concentration of JZP-458 deployed in the assay was 0.1 mIU/ml for SUDHL2 and 100 or 316 mIU/mL for other lines, the top concentrations of venetoclax used were 31623, 10000, 316, and 31.6 nM, Rituximab 3.2 ug/mL, Ibrutinib 31623, 10000, and 31.6 nM, vincristine 100, 31.6, and 10 nM, sirolimus 31623, 10000, 100, 31.6, and 10 nM with a √10 dilution series in the matrix (see FIGS. 13, 14, 15, and 17). The percent viability for each drug either alone or in combination was determined relative to control samples and the Bliss independence scores for each combination were determined as described (Liu, Q., et al., Stat Biopharm. Res. 10: 112; 2018). For each cell line for a given combination, individual scores within the matrix were summed to give a Bliss sum score for each cell line as described previously (Liu et al., 2018).

Cell Culture: Cell culture medium: RPMI 1640 medium (#2198761, Thermo Fisher) supplemented with 10% fetal calf serum (#S00AZ, BioWest) and 1% pen/strep (#15140-122, Thermo Fisher). At day-1, 900 μl cell suspension was seeded in 24-well plates (#662165, Greiner Bio-One) at a density of 128,000 cells/well. At day 0, 100 μl compound was added in three different concentrations to reach a final concentration of 0.316×IC50, 1×IC50, and 3.16×IC50 (unless indicated otherwise). To the control samples, 100 μl 20 mM HEPES+3.16% DMSO was added instead of compound. When the compound is prepared in HEPES instead of DMSO, 20 mM HEPES without DMSO was added to the cells. The cells were collected at 6 hours, 24 hours and 48 hours after addition of the compound and were transferred to a 96-well deep well plate and centrifuged for 5 min at 340×g. Prior to the collection of the cells microscopic pictures were taken from the untreated (control) cells. After removing the medium, 200 μl RNA protect (#76526, Qiagen) was added to the cell pellet and the cells were stored at −80° C.

RNA Isolation and cDNA Synthesis

RNA isolation: Total RNA was isolated with the SV96 Total RNA Isolation System kit (#Z3505, Promega) according to manufacturer's instructions. The procedure includes a ‘RNAse free DNAse’ gDNA removal step.

Determining the RNA concentration: RNA concentrations were determined with the RiboGreen (#R11490, Thermo Fisher) kit according to manufacturer's instructions.

cDNA synthesis: RNA was reverse transcribed into cDNA using the Quantitect Reverse Transcript Kit (#205313, Qiagen) according to manufacturer's instructions. This procedure includes a second ‘RNAse free DNAse’ gDNA removal step, ensuring the complete removal of gDNA from the samples. For each time point (6 h, 24 h, or 48 h) identical volumes of RNA were used for cDNA synthesis. After synthesis, cDNA is diluted with nuclease free water to obtain approximately similar cDNA concentrations per sample.

qPCR: 1) 50° C. for 10 min (Uracil-DNA glycosylase (UDP) step to remove all potential traces of amplicon DNA from previous qPCRs); 2) 5 min UDP inactivation/denaturation at 95° C.; 3) 40 cycles of 10s denaturation at 95° C., 20s annealing at 60° C., and 30s extension at 72° C.; and 4) Dissociation curve to determine the melting temperature of the qPCR amplicon.

qPCR Data Analysis: Cq and Tm raw data values are determined by the BioRad CFX manager software from the PCR machine. For every PCR sample that produced a Cq value the corresponding melting temperature of the PCR amplicon (Tm) was checked to identify samples that generated dual or a specific PCR products. A PCR product is regarded a specific when its Tm is >0.5° C. different from that the Tm of the specific PCR amplicon, in this case the Cq value is removed and not used for further data analysis. Cq values were than normalized by subtracting the (mean) Cq value of the two housekeeping genes (ACTB & RPS18) from the Cq value of the gene of interest. The normalized value is called DCq. The Cq difference between sample and the control (DDCq) was calculated by subtracting DCq control from DCq sample. The fold difference in expression between the control (no compound) and the compound treated sample is than calculated according the 2\hat{ }-DDCq method.

### Results

### Combination Results for L-asparaginase and BCL-XL

According to FIG. 1, a combination of JZP458+A1331852 (BCL-XL inhibitor) was found to be synergistic in all 10 cell lines in an in vitro study using a Bliss Assay. A positive Bliss Sum score is indicative of synergy with a score >150 being strong synergy. FIG. 5 shows synergy in a combination of JZP-458 with a secondary BCL-XL inhibitor A-1155463.

FIG. 9 shows strong synergy of L-asparaginase and A-1331852 treatment based on Bliss score when compared with L-asparaginase combined with bortezomib. FIG. 9 also shows synergy using the combination of JZP-458+BCL-XL inhibitor A-1331852 in a variety of cell lines. FIG. 1 shows another set of data for the combination of JZP-458+BCL-XLi (A-1331852) in various cell lines. The data shows that Solid tumors are more dependent on BCL-XL. SCLC has a spectrum of dependence on BCL-2/BCL-XL. 7/10 lines demonstrate combination synergy above threshold. Lines were chosen agnostic of BCL-XL expression. FIG. 1 shows the combination of JZP-458+BCL-XL inhibitor A-1331852 in three independent sets of experiments.

### Combination Results for L-asparaginase and BCL-2 Inhibitors

FIG. 2 shows the L-asparaginase and venetoclax combination was tested in a number of different cancer cell lines, including small cell lung cancer (SCLC) cell line NCI-H146, pancreatic cancer cell line MiaPaCa-2, ovarian cancer cell line OVCAR-3, sarcoma cell line SW982, breast cancer cell line MDA-MB-468, breast cancer cell line Hs578T, gastric cancer cell line KATO III, small cell lung cancer (SCLC) cell line NCI-H69, breast cancer cell line MDA-MB-453, and breast cancer cell line MDA-MB-231. NCI-H146, MiaPaCa-2 and OVCAR-3 showed some synergy and MDA-MB-468, MDA-MB-231, and NCI-H69 showed strong synergy.

FIG. 13 shows synergy of venetoclax with JZP458 in DLBCL line (ABC-type). The average CI50 for all ratios is 0.75. This indicates synergy of venetoclax with JZP458. FIG. 17 shows synergy of JZP458 venetoclax in lymphoma cell lines (BLISS matrix). FIG. 18 shows the tolerability of JZP341 in combination with venetoclax in naïve SCID mice. FIG. 2 shows the combination of JZP-458+Venetoclax (BCL-2i) in cell lines shows synergy in small cell lung cancer (SCLC) cell line NCI-H146, pancreatic cancer cell line MiaPaCa-2, sarcoma cell line SW982, breast cancer cell line MDA-MB-468, breast cancer cell line Hs578T, and gastric cancer cell line KATO III. Strong synergy was demonstrated in NCI-H69, a small cell lung cancer (SCLC) cell line, and MDA-MB-231, a breast cancer cell line.

### Combination Results for L-asparaginase and BCL-XL and BCL-2 Inhibitors

FIG. 3 shows testing of JZP458 (L-asparaginase)+Navitoclax (BCL-2/BCL-XL dual inhibitor) which demonstrates synergy in a number of different cancer cell lines, including small cell lung cancer (SCLC) cell line NCI-H146, pancreatic cancer cell line MiaPaCa-2, ovarian cancer cell line OVCAR-3, sarcoma cell line SW982, breast cancer cell line MDA-MB-468, breast cancer cell line Hs578T, gastric cancer cell line KATO III, small cell lung cancer (SCLC) cell line NCI-H69, breast cancer cell line MDA-MB-453, and breast cancer cell line MDA-MB-231. FIG. 14 shows synergy of rapamycin with JZP458 in DLBCL line (ABC-type).

Combination Results for L-asparaginase and mTOR Inhibitors

Data in FIG. 8 shows that mTOR inhibitors, specifically everolimus, show an increased Bliss sum score when compared with PI3K inhibitors and MEK inhibitors. FIG. 17 shows synergy of rapamycin with JZP458 in DLBCL line (ABC-type). The average of CI50 is 0.57 indicates synergy of rapamycin with JZP458. FIGS. 18 and 19 show tolerability of JZP341 in combination with sirolimus in naïve SCID mice.

### Combination Results for L-asparaginase and CD20 Inhibitors

Data showing combination therapies involving an L-asparaginase and a CD20 inhibitor can be found in FIGS. 18, and 21. FIG. 15 shows synergy of rituximab with JZP458 in DLBCL line (THL). The CI50 average of 0.46 indicates strong synergy of rituximab with JZP458. FIG. 21 shows synergy of JZP458 with rituximab, JZP458 with venetoclax, and JZP458 with ibrutinib in lymphoma lines (BLISS matrix). FIG. 18 and FIG. 19 shows tolerability of L-asparaginase (JZP341) in combination with rapamycin, venetoclax, and R-CHOP constituents in naïve SCID mice.

### Combination Results for L-asparaginase and BTK Inhibitors

FIG. 21 shows synergy of JZP458 with ibrutinib in lymphoma lines (BLISS matrix).

### Combination Results for L-asparaginase and Glutaminase Inhibitors

FIG. 4 shows JZP458 combined with CB-839. Synergy was found for sarcoma cell line SW982 and breast cancer cell line MDA-MB-231. Synergy was indicated for small cell lung cancer (SCLC) cell line NCI-146, breast cancer cell line MDA-MB-468, and gastric cancer cell line KATOIII. The effects of the combination were independent of ASNS expression levels.

### Results in Lymphoma Cell Lines

Sensitivity to asparaginase and 11 chemotherapeutic agents (carfilzomib, doxorubicin, rapamycin, ibrutinib, cyclophosphamide, prednisolone, metformin, venetoclax, navitoclax, rituximab and vincristine) determined in 8 lymphoma lines, including diffuse large B-cell and Burkitt lymphomas. The investigated lymphoma lines are sensitive to asparagine depletion within the clinically achievable range. Expression of decisive genes (BCL2, BCL6, BCL2L1, MYC, ASNS) determined in 8 lymphoma lines (FIGS. 11 and 12). Increased expression of BCL2 served as marker for resistance of ibrutinib and rapamycin in triple hit lymphoma (FIGS. 11 and 12). Increased expression of ASNS served as marker for resistance of carfilzomib and asparaginase in Burkitt lymphoma (FIGS. 11 and 12). Identified combination agents that enhanced asparaginase activity in vitro include synergy of JZP458 and rituximab, venetoclax and ibrutinib in lymphoma lines (DLBCL) (see FIG. 13, FIG. 15, and FIG. 17). Apoptosis and autophagy pathways induced by asparaginase suggests venetoclax, ibrutinib and rituximab as combination partners in liquid tumors, particularly diffuse large B-cell lymphomas (DLBCL) (FIG. 17). In vivo tolerability study of long acting Erwinia Asparaginase. JZP341* shows that JZP341* at 150 U/kg in combination with venetoclax and R-CHOP constituents were tolerated (FIG. 18 and FIG. 19). JZP341* is long acting asparaginase (half life t½≅16 hrs) and has the same mechanism of action as short acting asparaginase JZP458 (t½≅2.13 hrs).

### NRAS Mutations

FIGS. 10A, 10B, and 10C show liquid tumors with NRAS mutations appear to be more sensitive to asparaginase. Across the panel sensitivity difference due to NRAS mutations is driven by liquid tumors. NRAS mutations appear in FLT3 resistant AML. FLT3 treatment emergent RAS/MAPK pathway mutations in 37% patients. Activating mutations in NRAS in 32%. KRAS mutations in 7%. No patients at baseline had detectable NRAS/KRAS mutations.

### Sensitivity to Cell Lines

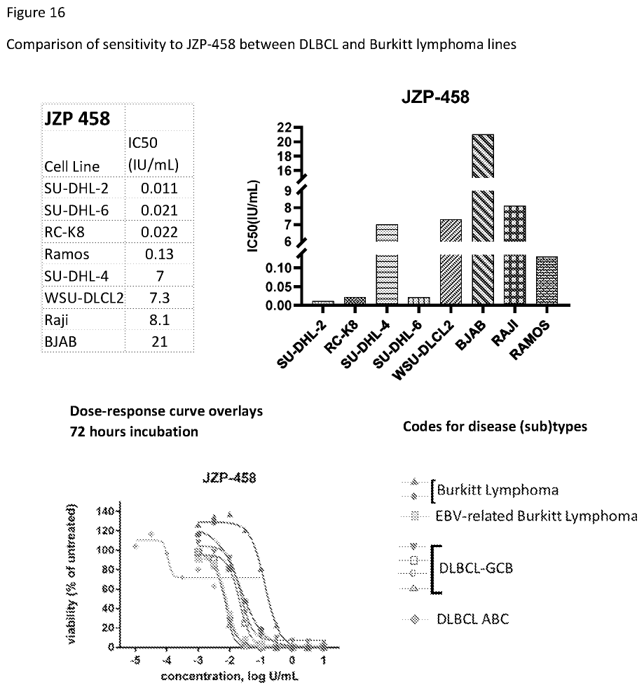

FIG. 16 shows strong sensitivity of DLBCL and Burkitt lymphoma lines to JZP458. ABC-DLBCL lines and one of GCB-DLBCL lines are very sensitive to JZP458 in the range of 0.011-7.3 IU/mL. Dose-response curve overlays-72 hrs incubation.

### ASNS and BCL-2 Expression

A subset of cell lines are ASNS* dependent for survival (pDrive in vitro). *ASNS used as a surrogate for Asparagine depletion. Lung cancers stand out as non-dependent on ASNS depletion. Cumulative frequency of ASNS dependence in NSCLC is 5%. FIG. 6 shows most indications fall within the same range of sensitivity. FIG. 7 shows combinations tested for ASNS expression. The choice of inhibitors was informed by pathway analysis and those affecting asparagine synthetase expression. FIG. 11 shows decreased BCL2 expression as prognostic marker for DLBCL lines (ABC and GCB type). JZP458 and most chemotherapeutic agents decrease BCL2 expression. FIG. 12 shows increased BCL2 expression and increased ASNS expression as resistance marker for DLBCL lines (triple hit lymphoma) and Burkitt lymphoma. Ibrutinib and rapamycin significantly increase BCL2 expression in triple hit lymphoma line WSU-DLCL2 and carfilzomib and JZP458 ASNS in Burkitt lymphoma lines.

### Example 2: Combination Treatments with Pasylated L-asparaginase

Synergistic activity between pasylated L-asparaginase and A1331852 (a BCL-XL inhibitor), venetoclax (a BCL-2 inhibitor), and navitoclax (a dual BCL-XL/BCL-2 inhibitor) was determined for 10 cancer cell lines. Cells were plated in 384-well plates in 45 μl and incubated as outlined in Example 1. For each cell line, the optimal cell density was determined to obtain an optimal assay window. Plated cells were incubated in a humidified incubator with 5% CO₂ at 37° C. 24 hours post-plating, 5 μl of compound dilution was added to each well and the plates were further incubated for another 72 hours after which time 25 μl of ATPlite 1 Step solution was added to each well.

Luminescence was recorded on an Envision multimode reader as outlined in Example 1. For determination of Bliss independence scores, pasylated L-asparaginase and either A1331852, venetoclax, and navitoclax were added in increasing concentrations in a 6×6 matrix. The percent viability for each drug either alone or in combination was determined relative to control samples and the Bliss independence scores for each cell line and combination treatment were determined as described in Example 1.

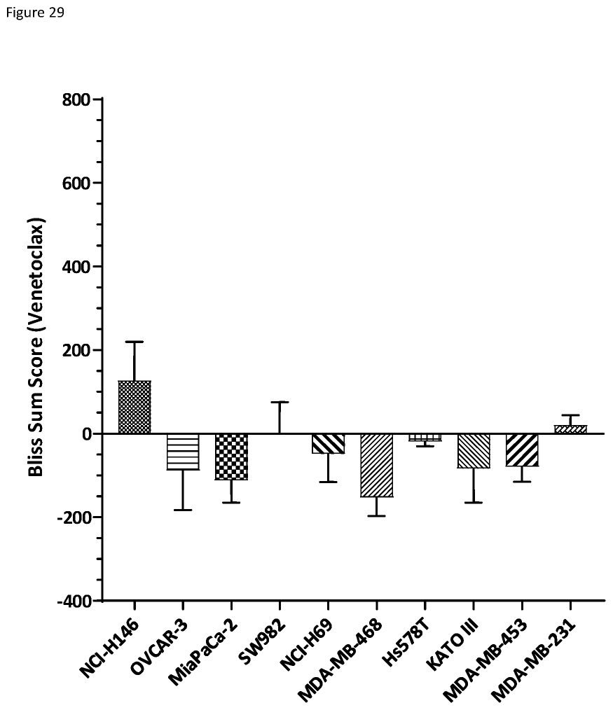

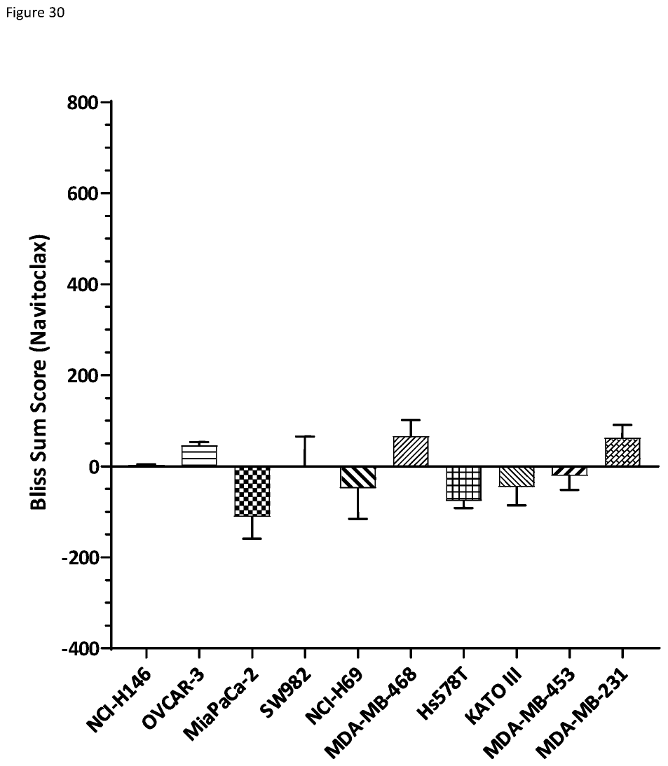

Cancer treatment synergy between pasylated L-asparaginase and the BCL-XL, BCL-2, and BCL-XL/BCL-2 dual inhibitors was observed for multiple cancer types. For A-1331852, positive Bliss scores were observed for ovarian (OVCAR-3), synovial (SW982), lung cancer (NCI-H69), breast cancer (MDA-MB-468, Hs578T, and MDA-MB-231), and gastric cancer (KATO III) cell lines (FIG. 28). For venetoclax, positive Bliss scores were observed for lung cancer (NCI-H146) and breast cancer (MDA-MB-231) cell lines (FIG. 29). For navitoclax, positive Bliss scores were observed for ovarian (OVCAR-3) and breast cancer (MDA-MB-468 and MDA-MB-231) cell lines.

While embodiments and applications of the present disclosure have been described in some detail by way of illustration and example, it would be apparent to those of skill in the art that many additional modifications would be possible without departing from the inventive concepts contained herein. All references cited herein are hereby incorporated in their entirety.

Although the invention has been described with reference to the above examples, it will be understood that modifications and variations are encompassed within the spirit and scope of the invention. Accordingly, the invention is limited only by the following claims.

1. A method of treating cancer in a patient comprising administering to the patient (a) an effective amount of an L-asparaginase and (b) an anti-cancer agent,
wherein the anti-cancer agent is (i) an inhibitor of BCL-XL, (ii) an inhibitor of BCL-2, (iii) an inhibitor of both BCL-XL and BCL-2, (iv) a mTOR inhibitor, (v) a CD20 inhibitor, or (vi) a BTK inhibitor, thereby treating cancer in the patient.

2. The method of claim 1, wherein the inhibitor is selected from the group consisting of: a small molecule, an antibody, an antibody-drug conjugate with a payload, a dendrimer with a payload, a prodrug, and a proteolysis targeting chimera (PROTAC).

3. The method of claim 1, wherein the BCL-XL inhibitor is selected from the group consisting of: A-1155463, A-1331852, WEHI-539, WEHI-539 HCl, BH3I-1, A-1293102, DT2216, XZ424, XZ739, PZ15227, PROTAC 1, and ABBV-155.

4-5. (canceled)

6. The method of claim 1, wherein the BCL-2 inhibitor is selected from the group consisting of: venetoclax (ABT-199), S55746, BDA-366, oblimersen (G3139), obatoclax, obatoclax mesylate (GX15-070), HA14-1, mifepristone (RU486), TCPOBOP, cinobufagin, nodakenetin (NANI), and motixafortide (BL-8040).

7-8. (canceled)

9. The method of claim 1, wherein the inhibitor of both BCL-XL and BCL-2 is selected from the group consisting of: navitoclax (ABT-263), ABT-737, sabutoclax, gossypol, (R)-(−)-gossypol acetic acid, TW-37, gambogic acid, 2-Methoxy-antimycin A, berberine chloride (NSC 646666), berberine chloride hydrate, APG-1252, AZD-0466, BM-1197, AZD4320, and pelcitoclax (APG-1252).

10-11. (canceled)

12. The method of claim 1, wherein the mTOR inhibitor is selected from the group consisting of: rapamycin (sirolimus), rapalogs (rapamycin derivatives), temsirolimus (CCI-779), everolimus (RAD001), and ridaforolimus (AP-23573).

13-14. (canceled)

15. The method of claim 1, wherein the CD20 inhibitor is selected from the group consisting of: rituximab, ofatumumab, ublituximab, ocrelizumab, obinutuzumab, ocaratuzumab, ibritumomab tiuxetan, tositumomab, TRU-015, IMMU-106, and R-CHOP.

16-17. (canceled)

18. The method of claim 1, wherein the BTK inhibitor is selected from the group consisting of: ibrutinib, zanubrutinib, and acalabrutinib.

19. The method of claim 1, wherein the patient has an NRAS mutation.

20. The method of claim 1, wherein the L-asparaginase is a recombinant L-asparaginase having at least about 80%, 85%, 90%, 91%, 92%, 93%, 94%, 95%, 96%, 97%, 98%, 99%, or 100% sequence identity to the amino acid of SEQ ID NO: 1.

21. (canceled)

22. The method of claim 1, wherein the L-asparaginase is a tetramer, and wherein each monomer of the tetramer has a sequence identity of at least 95 percent to SEQ ID NO: 1.

23. (canceled)

24. The method of claim 1, wherein the patient exhibited hypersensitivity to the E-coli.-derived asparaginase, a PEGylated form thereof, or to an Erwinia asparaginase.

25. The method of claim 24, wherein the patient has terminated E-coli.-derived asparaginase therapy.

26. The method of claim 1, wherein the patient is an adult or is a pediatric patient.

27. (canceled)

28. The method of claim 1, wherein the L-asparaginase demonstrates less than 6% aggregation or less than 1% aggregation.

29. (canceled)

30. The method of claim 1, wherein the L-asparaginase is non-lyophilized, or wherein the L-asparaginase is unfunctionalized.

31. The method of claim 1, wherein the L-asparaginase is recombinantly produced in Pseudomonas fluorescens.

32. The method according to claim 1, wherein a nadir serum asparaginase activity (NSAA) assay as measured from a serum sample from the human subject equals or exceeds 0.1 IU/mL after administration after treatment with the L-asparaginase.

33. The method according to claim 1, wherein the L-asparaginase is conjugated with a PEG moiety and/or with a proline- or alanine-containing peptide.

34. (canceled)

35. The method according to claim 1, wherein the cancer is acute lymphoblastic leukemia (ALL), er lymphoblastic lymphoma (LBL), acute myeloid leukemia (AML) or lymphoma.

36. The method according to claim 1, wherein the cancer is a solid cancer selected from the group consisting of colorectal cancer (CRC), breast cancer, pancreatic cancer, glioblastoma, lung cancer, small cell lung cancer (SCLC), ovarian cancer, sarcoma, and gastric cancer.

37. The method according to claim 36, wherein the CRC is Wnt-negative CRC.

38. (canceled)

39. The method according to claim 35, wherein the AML is R/R AML or FLT3 resistant AML.

40-41. (canceled)

42. The method according to claim 35, wherein the lymphoma is Burkitt lymphoma, DLBCL or B cell lymphoma.

43-45. (canceled)

46. The method according to claim 36, wherein the breast cancer is TNBC.

47-53. (canceled)

54. The method of claim 1, wherein the method comprises using ASNS and/or BCL2 expression as a prognostic marker to determine treatment.

55. The method of claim 1, further comprising administering a glutaminase inhibitor to the patient.

56. The method of claim 55, wherein the glutaminase inhibitor is CB-839.

57. (canceled)

58. A method of treating NRAS mutant acute myeloid leukemia (AML) in a patient comprising administering to the patient an effective amount of an L-asparaginase.

59. A method of treating solid cancer in a patient comprising administering to the patient an effective amount of an L-asparaginase in combination with a BCL-XL inhibitor.

60-61. (canceled)

62. The method of claim 59, wherein the L-asparaginase is unfunctionalized.

63. (canceled)
